(19) United States
(12) Patent Application Publication
(10) Pub. No.: US 2019/0185457 A1
(43) Pub. Date: Jun. 20, 2019

(54) HETEROCYCLIC COMPOUND, COMPOSITION INCLUDING THE SAME, AND ORGANIC LIGHT-EMITTING DEVICE INCLUDING THE HETEROCYCLIC COMPOUND

(71) Applicant: Samsung Electronics Co., Ltd., Suwon-si (KR)

(72) Inventors: Shiro IRISA, Kanagawa (JP); Keisuke KORAI, Kanagawa (JP); Mitsunori ITO, Kanagawa (JP)

(21) Appl. No.: 16/220,031

(22) Filed: Dec. 14, 2018

(30) Foreign Application Priority Data
Dec. 19, 2017 (JP) 2017-242524
Jul. 16, 2018 (KR) 10-2018-0082207

(51) Int. Cl. C07D 403/10; C07D 401/14; C07D 403/14; C07D 405/14; C07D 409/14; C07D 471/04; C07D 239/04; C07D 495/04; H01L 51/00; H01L 51/50

(52) U.S. Cl. CPC C07D 403/10; H01L 51/5072; C07D 403/14; C07D 405/14; C07D 409/14; C07D 471/04; C07D 239/04; C07D 495/04; H01L 51/0072; H01L 51/0073; H01L 51/0074; H01L 51/0067; H01L 51/0058; H01L 51/006; H01L 51/0034; H01L 51/5092; H01L 51/5096; H01L 51/5016; H01L 51/5056; C07D 401/14

(57) ABSTRACT

A heterocyclic compound represented by Formula 1:

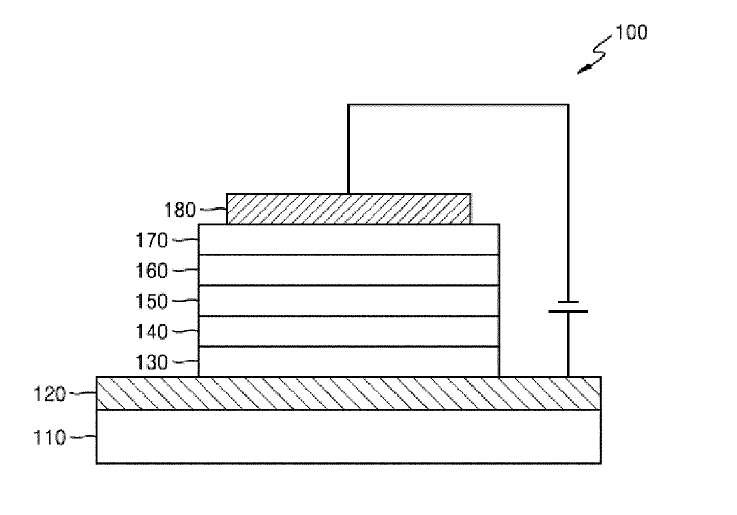

(A)ₙ-L, Formula 1

wherein, in Formula 1, A, L, and n are the same as described in the specification.

### CROSS-REFERENCE TO RELATED APPLICATIONS

This application claims priority to Japanese Patent Application No. 2017-242524, filed on Dec. 19, 2017, in the Japanese Patent Office and Korean Patent Application No. 10-2018-0082207, filed on Jul. 16, 2018, in the Korean Intellectual Property Office, and all the benefits accruing therefrom under 35 U.S.C. § 119, the contents of which are incorporated herein in their entireties by reference.

### BACKGROUND

### 1. Field

One or more embodiments relate to a heterocyclic compound, a composition including the same for an organic light-emitting device, and an organic light-emitting device including the heterocyclic compound.

### 2. Description of the Related Art

Organic light-emitting devices (OLEDs) are self-emission devices that produce full-color images, and that also have wide viewing angles, high contrast ratios, short response times, and excellent characteristics in terms of brightness, driving voltage, and response speed, compared to devices in the art.

In an example, an organic light-emitting device includes an anode, a cathode, and an organic layer disposed between the anode and the cathode, wherein the organic layer includes an emission layer. A hole transport region may be disposed between the anode and the emission layer, and an electron transport region may be disposed between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. These excitons transit from an excited state to a ground state, thereby generating light.

Various types of organic light emitting devices are known. However, there still remains a need in OLEDs having low driving voltage, high efficiency, high brightness, and long lifespan.

### SUMMARY

Aspects of the present disclosure provide a heterocyclic compound, a composition including the same, and an organic light-emitting device including the heterocyclic compound.

The organic light-emitting device including the heterocyclic compound may have high current efficiency and a long lifespan. In addition, the heterocyclic compound may have characteristics suitable for use in solution coating.

Additional aspects will be set forth in part in the description which follows and, in part, will be apparent from the description, or may be learned by practice of the presented embodiments.

An aspect provides a heterocyclic compound represented by Formula 1:

(A)₁-L. Formula 1

In Formula 1, A may be a group in which one hydrogen atom is excluded from a compound represented by Formula 1-1 or a group in which one hydrogen atom is excluded from a compound represented by Formula 1-2,

n may be an integer from 1 to 8,

L may be hydrogen, a single bond, or an n-valent linking group, and

when n is 1, L may be hydrogen:

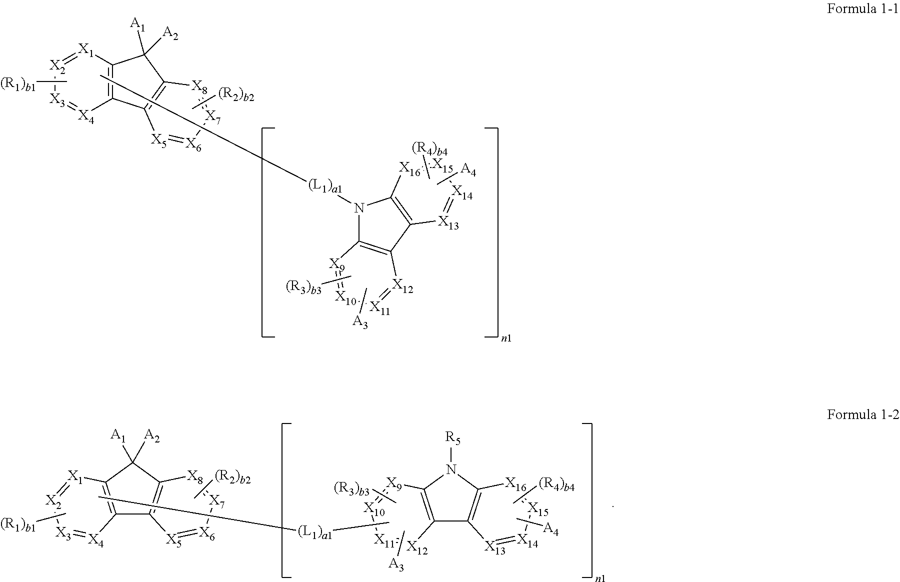

In Formulae 1-1 and 1-2,

X₁ to X₁₆ may each independently be N or C,

at least one selected from X₁ to X₈ may be carbon linked to -(L₁)ₐ₁-,

in Formula 1-1, one selected from X₉ to X₁₂ may be carbon linked to A₃, and one selected from X₁₃ to X₁₆ may be carbon linked to A₄,

in Formula 1-2, one selected from X₉ to X₁₂ may be carbon linked to -(L₁)ₐ₁-, and one selected from X₁₃ to X₁₆ may be carbon linked to A₄,

A₁ and A₄ may each independently be selected from hydrogen, a group represented by *-(L₂)ₐ₂-R_{ET}, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group,

at least one selected from A₃ and A₄ may be a group represented by *-(L₂)ₐ₂-R_{ET},

A₁ and A₂ may optionally be linked to form a saturated ring or an unsaturated ring,

L₁ and L₂ may each independently be selected from a substituted or unsubstituted C₅-C₃₀ carbocyclic group and a substituted or unsubstituted C₁-C₃₀ heterocyclic group,

a1 and a2 may each independently be an integer from 0 to 3, wherein, when a1 is 0, *-(L₁)ₐ₁-*′ may be a single bond, and when a2 is 0, *-(L₂)ₐ₂-*′ may be a single bond,

R_{ET} may be a substituted or unsubstituted C₁-C₆₀ nitrogen-containing heterocyclic group,

R₁ to R₅ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), —B(Q₆)(Q₇), —P(═O)(Q₈)(Q₉), and —C(═O)(Q₁₀),

b1 and b2 may each independently be an integer from 0 to 4, and b3 and b4 may each independently be an integer from 0 to 3,

n1 may be an integer from 1 to 8,

wherein, in Formula 1-1, a substituted or unsubstituted pyridinyl group, a substituted or unsubstituted pyrimidinyl group, a substituted or unsubstituted pyrazinyl group, a substituted or unsubstituted pyrazinyl group, and a substituted or unsubstituted triazinyl group are excluded from R_{ET} when A₁ and A₂ are each independently a substituted or unsubstituted C₁-C₆₀ alkyl group,

at least one substituent of the substituted C₁-C₆₀ nitrogen-containing heterocyclic group, the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, —CD₃, CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —P(═O)(Q₁₈)(Q₁₉), and —C(═O)(Q₂₀);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —P(═O)(Q₂₈)(Q₂₉), and —C(═O)(Q₃₀); and

—N(Q₃₁)(Q₃₂), —Si(Q₃₃)(Q₃₄)(Q₃₅), —B(Q₃₆)(Q₃₇), —P(═O)(Q₃₈)(Q₃₉), and —C(═O)(Q₄₀), and

Q₁ to Q_{10,} Q₁₁ to Q_{20,} Q₂₁ to Q_{30,} and Q₃₁ to Q₄₀ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one selected from a C₁-C₆₀ alkyl group and a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

Another aspect provides a composition including the heterocyclic compound.

Another aspect provides an organic light-emitting device including:

a first electrode;

a second electrode; and

an organic layer disposed between the first electrode and the second electrode,

wherein the organic layer includes an emission layer, and

wherein the organic layer includes at least one heterocyclic compound.

### BRIEF DESCRIPTION OF THE DRAWING

These and/or other aspects will become apparent and more readily appreciated from the following description of the embodiments, taken in conjunction with the FIGURE which is a schematic view of an organic light-emitting device according to an embodiment.

### DETAlLED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

Heterocyclic compound

A heterocyclic compound according to an embodiment is represented by Formula 1:

(A)ₙ-L. Formula 1

In Formula 1, A may be a group in which one hydrogen atom is excluded from a compound represented by Formula 1-1 or a group in which one hydrogen atom is excluded from a compound represented by Formula 1 -2.

In Formula 1, n may be an integer from 1 to 8.

In an embodiment, n may be an integer from 1 to 4. For example, n may be 1 or 2.

In Formula 1, L may be hydrogen, a single bond, or an n-valent linking group. When n is 1, L is hydrogen.

In an embodiment, L may be hydrogen, a single bond, a substituted or unsubstituted C₁-C₆₀ alkylene group, a substituted or unsubstituted C₆-C₆₀ arylene group, —O—, —S—, or —N(R₁₀₁)—.

R₁₀₁ may be hydrogen, deuterium, a substituted or unsubstituted C₁-C₆₀ alkyl group, a substituted or unsubstituted C₆-C₆₀ aryl group, or a substituted or unsubstituted C₁-C₆₀ heteroaryl group.

In an embodiment, L may be selected from a methylene group, an ethylene group, an n-propylene group, an n-butylene group, a phenylene group, a biphenylene group, a terphenylene group, and a fluorenylene group.

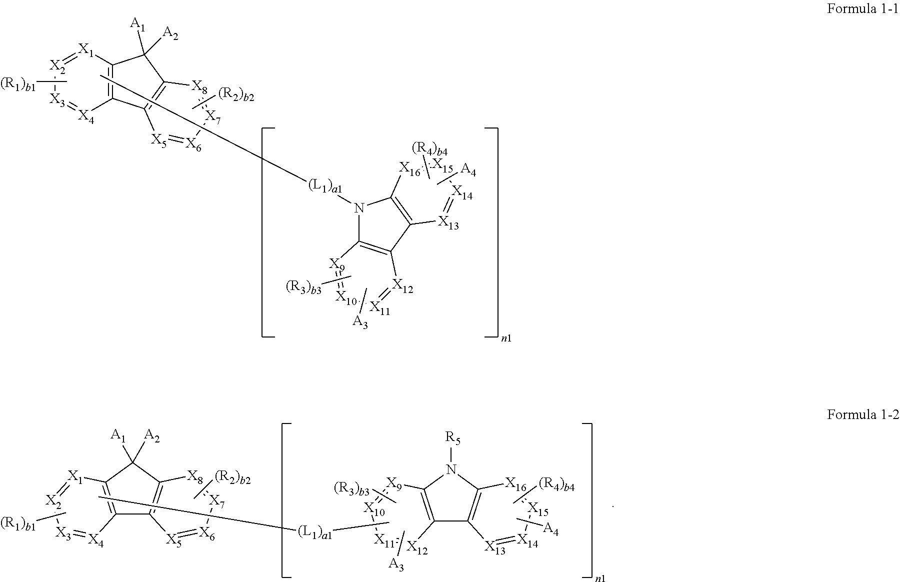

In Formulae 1-1 and 1-2, X₁ to X₁₆ may each independently be N or C.

In an embodiment, in Formulae 1-1 and 1-2, at least one selected from X₁ to X₁₆ may be N.

In an embodiment, in Formulae 1-1 and 1-2, X₄ or X₅ may be N, or X₄ and X₅ may each be N.

In Formulae 1-1 and 1-2, at least one selected from X₁ to X₈ may be carbon linked to —(L₁)ₐ₁—.

In Formula 1-1, one selected from X₉ to X₁₂ may be carbon linked to A₃, and one selected from X₁₃ to X₁₆ may be carbon linked to A₄.

In Formula 1-2, one selected from X₉ to X₁₂ may be carbon linked to —(L₁)ₐ₁—, and one selected from X₁₃ to X₁₆ may be carbon linked to A₄.

In Formulae 1-1 and 1-2, A₁ and A₄ may each independently be selected from hydrogen, a group represented by *—(L₂)ₐ₂—R_{ET}, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

At least one selected from A₃ and A₄ may be a group represented by *—(L₂)ₐ₂—R_{ET}.

In an embodiment, in Formula 1-1, a position of carbons to which A₃ is linked may be asymmetric to a position of carbons to which A₄ is linked.

In one or more embodiments, in Formula 1-1, X₁₂ may be carbon linked to A₃, X₁₄ may be carbon linked to A₄, A₃ is a group represented by *—(L₂)ₐ₂—R_{ET}, and A₄ may be a substituted or unsubstituted C₆-C₆₀ aryl group.

In one or more embodiments, in Formula 1-1, X₁₂ may be carbon linked to A₃, X₁₄ may be carbon linked to A₄, A₃ may be a group represented by *—(L₂)ₐ₂—R_{ET}, A₄ may be a phenyl group, a biphenyl group, a terphenyl group, or a tetraphenyl group.

In an embodiment, in Formula 1-2, a position of carbons to which —(L₁)ₐ₁— is linked may be asymmetric to a position of carbons to which A₄ is linked.

In one or more embodiments, in Formula 1-2, X₁₁ may be carbon linked to —(L₁)ₐ₁—, X₁₃ may be carbon linked to A₄, and A₄ may be a group represented by *—(L₂)ₐ₂—R_{ET}.

In an embodiment, in Formulae 1-1 and 1-2, A₁ to A₄ may each independently be selected from:

hydrogen, a group represented by *—(L₂)ₐ₂—R_{ET}, a methyl group, an ethyl group, an n-propyl group, an iso-propyl group, an n-butyl group, an iso-butyl group, a sec-butyl group, a tert-butyl group, an n-pentyl group, an iso-pentyl group, a tert-pentyl group, a neo-pentyl group, a 1,2-dimethylpropyl group, an n-hexyl group, an iso-hexyl group, a 1,3-dimethylbutyl group, a 1-iso-propylpropyl group, a 1,2-dimethylbutyl group, an n-heptyl group, a 1,4-dimethylpentyl group, a 3-ethylpentyl group, a 2-methyl-1-iso-propylpropyl group, a 1-ethyl-3-methylbutyl group, an n-octyl group, a 2-ethylhexyl group, a 3-methyl-1-iso-propylbutyl group, a 2-methyl-1- iso-propyl group, a 1-tert-butyl-2-methylpropyl group, an n-nonyl group, a 3,5,5-trimethyldecyl group, an n-decyl group, an isodecyl group, an n-undecyl group, a 1-methyldecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, an n-eicosyl group, an n-heneicosyl group, an n-docosyl group, an n-tricosyl group, an n-tetracosyl group, a methoxy group, an ethoxy group, a propoxy group, an iso-propoxy group, an n-butoxy group, an iso-butoxy group, a sec-butoxy group, a tert-butoxy group, an n-pentoxy group, an iso-pentoxy group, a tert-pentoxy group, a neo-pentoxy group, an n-hexyloxy group, an iso-hexyloxy group, a heptyloxy group, an octyloxy group, a nonyloxy group, a decyloxy group, an undeoxy group, a dodecyloxy group, a tridecyloxy group, a tetradecyloxy group, a pentadecyloxy group, a hexadecyloxy group, a heptadecyloxy group, an octadecyloxy group, a 2-ethylhexyloxy group, a 3-ethylpentyloxy group, a phenoxy group, a 1-naphthyloxy group, a 2-naphthyloxy group, a 2-azulenyloxy group, a 2-furanyloxy group, a 2-thienyloxy group, a 2-indoleyloxy group, a 3-indoleyloxy group, a 2-benzofuriloxy group, a 2-benzothienyloxy group, an N-methylamino group, an N-ethylamino group, an N-propylamino group, an N-iso-propylamino group, an N-butylamino group, an N-iso-butylamino group, an N-sec-butylamino group, an N-tert-butylamino group, an N-pentylamino group, an N-hexylamino group, an N.N-dimethylamino group, an N,N-diethylamino group, an N,N-dipropylamino group, an N,N-di-iso-propylamino group, an N,N-dibutylamino group, an N,N-di-iso-butylamino group, an N,N-dipentylamino group, and an N,N-dihexylamino group;

a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group; and

a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group, each substituted with at least one selected from deuterium, —F, —CI, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

In Formulae 1-1 and 1-2, A₁ and A₂ may optionally be linked to form a saturated ring or an unsaturated ring.

In an embodiment, in Formulae 1-1 and 1-2, A₁ and A₂ may be linked to form a group represented by Formula 9:

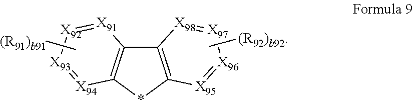

In Formula 9,

X₉₁ to X₉₈ may each independently be N or C,

R₉₁ to R₉₂ may each independently be selected from hydrogen, deuterium, —F, —CI, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted heterocycloalkyl group, a substituted or unsubstituted C₃-C₁₀ cycloalkenyl group, a substituted or unsubstituted heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), and —B(Q₆)(Q₇),

b91 to b92 may each independently be an integer from 1 to 4, and

* indicates a binding site to a neighboring atom.

In Formulae 1-1 and 1-2, L₁ and L₂ may each independently be selected from a substituted or unsubstituted C₅-C₃₀ carbocyclic group and a substituted or unsubstituted C₁-C₃₀ heterocyclic group.

In an embodiment, L₁ and L₂ may each independently be selected from:

a benzene group, a pentalene group, an indene group, a naphthalene group, an anthracene group, an azulene group, a heptalene group, an acenaphthylene group, a phenalene group, a fluorene group, a spiro-bifluorene group, a benzofluorene group, a dibenzofluorene group, a phenanthrene group, biphenyl group, terphenyl group, a triphenylene group, a fluoranthene group, a pyrene group, a chrysene group, a picene group, a perylene group, a pentaphene group, a pentacene group, a tetraphene group, a hexaphene group, a hexacene group, a rubicene group, a trinaphthalene group, a heptaphene group, a pyranthrene group, a pyridine group, a pyrazine group, a pyridazine group, a pyrimidine group, a triazine group, a quinoline group, an isoquinoline group, a quinoxaline group, a quinazoline group, a naphthyridine group, an acridine group, a phenazine group, a benzoquinoline group, a benzoisoquinoline group, a phenanthridine group, a phenanthroline group, a benzoquinone group, a coumarin group, an anthraquinone group, a fluorenone group, a furan group, a thiophene group, a silole group, a benzofuran group, a benzothiophene group, a benzosilole group, a dibenzofuran group, a dibenzothiophene group, a dibenzosilole group, a pyrrole group, an indole group, an isoindole group, a carbazole group, a benzocarbazole group, a dibenzocarbazole group, an imidazole group, a benzimidazole group, a pyrazole group, a triazole group, a tetrazole group, an indazole group, an oxazole group, an isoxazole group, a benzoxazole group, a benzoisoxazole group, a thiazole group, an isothiazole group, a benzothiazole group, a benzoisothiazole group, an imidazolinone group, a benzoimidazolinone group, an imidazopyridine group, an imidazopyrimidine group, an imidazophenanthridine group, a benzoimidazophenanthridine group, an azadibenzofuran group, an azacarbazole group, an azadibenzothiophene group, a diazadibenzofuran group, a diazacarbazole group, a diazadibenzothiophene group, a xanthone group, and a thioxanthone group; and

a benzene group, a pentalene group, an indene group, a naphthalene group, an anthracene group, an azulene group, a heptalene group, an acenaphthylene group, a phenalene group, a fluorene group, a spiro-bifluorene group, a benzofluorene group, a dibenzofluorene group, a phenanthrene group, biphenyl group, terphenyl group, a triphenylene group, a fluoranthene group, a pyrene group, a chrysene group, a picene group, a perylene group, a pentaphene group, a pentacene group, a tetraphene group, a hexaphene group, a hexacene group, a rubicene group, a trinaphthalene group, a heptaphene group, a pyranthrene group, a pyridine group, a pyrazine group, a pyridazine group, a pyrimidine group, a triazine group, a quinoline group, an isoquinoline group, a quinoxaline group, a quinazoline group, a naphthyridine group, an acridine group, a phenazine group, a benzoquinoline group, a benzoisoquinoline group, a phenanthridine group, a phenanthroline group, a benzoquinone group, a coumarin group, an anthraquinone group, a fluorenone group, a furan group, a thiophene group, a silole group, a benzofuran group, a benzothiophene group, a benzosilole group, a dibenzofuran group, a dibenzothiophene group, a dibenzosilole group, a pyrrole group, an indole group, an isoindole group, a carbazole group, a benzocarbazole group, a dibenzocarbazole group, an imidazole group, a benzimidazole group, a pyrazole group, a triazole group, a tetrazole group, an indazole group, an oxazole group, an isoxazole group, a benzoxazole group, a benzoisoxazole group, a thiazole group, an isothiazole group, a benzothiazole group, a benzoisothiazole group, an imidazolinone group, a benzoimidazolinone group, an imidazopyridine group, an imidazopyrimidine group, an imidazophenanthridine group, a benzoimidazophenanthridine group, an azadibenzofuran group, an azacarbazole group, an azadibenzothiophene group, a diazadibenzofuran group, a diazacarbazole group, a diazadibenzothiophene group, a xanthone group, and a thioxanthone group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

In Formulae 1-1 and 1-2, a1 and a2 may each independently be an integer from 0 to 3. When a1 is 0, *—(L₁)ₐ₁—*′ may be a single bond, and when a2 is 0, *—(L₂)ₐ₂—*′ may be a single bond.

R_{ET} may be a substituted or unsubstituted C₁-C₆₀ nitrogen-containing heterocyclic group.

In Formula 1-1, a substituted or unsubstituted pyridinyl group, a substituted or unsubstituted pyrimidinyl group, a substituted or unsubstituted pyrazinyl group, a substituted or unsubstituted pyrazinyl group, and a substituted or unsubstituted triazinyl group are excluded from R_{ET}, when A₁ and A₂ are each independently a substituted or unsubstituted C₁-C₆₀ alkyl group.

In an embodiment, R_{ET} may be selected from:

an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a benzophenanthrolinyl group, a phenazinyl group, a benzimidazolyl group, a naphthoimidazolyl group, a benzothiazolyl group, an isobenzothiazolyl group, a benzoxazolyl group, an isobenzoxazolyl group, a triazolyl group, a tetrazolyl group, an oxadiazolyl group, an imidazopyridinyl group, an imidazopyrimidinyl group, an imidazoquinolinyl group, an imidazoisoquinolinyl group, an azadibenzofuranyl group, a diazadibenzofuranyl group, an azadibenzothiophenyl group, a diazadibenzothiophenyl group, an azafluorenyl group, a diazafluorenyl group, an azaspirobifluorenyl group, and a diazaspirobifluorenyl group;

an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a benzophenanthrolinyl group, a phenazinyl group, a benzimidazolyl group, a naphthoimidazolyl group, a benzothiazolyl group, an isobenzothiazolyl group, a benzoxazolyl group, an isobenzoxazolyl group, a triazolyl group, a tetrazolyl group, an oxadiazolyl group, an imidazopyridinyl group, an imidazopyrimidinyl group, an imidazoquinolinyl group, an imidazoisoquinolinyl group, an azadibenzofuranyl group, a diazadibenzofuranyl group, an azadibenzothiophenyl group, a diazadibenzothiophenyl group, an azafluorenyl group, a diazafluorenyl group, an azaspirobifluorenyl group, and a diazaspirobifluorenyl group, each substituted with at least one selected from deuterium, —F, —Cl, —Br, —I, a hydroxyl group, a cyano group, a nitro group, an amidino group, a hydrazine group, a hydrazone group, a C₁-C₁₀ alkyl group, a C₁-C₁₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a triphenylenyl group, a fluoranthenyl group, a pyrenyl group, a chrysenyl group, a picenyl group, a perylenyl group, a benzosilolyl group, a dibenzofuranyl group, a dibenzothiophenyl group, a pyridinyl group, a pyrazinyl group, a pyrimidinyl group, a pyridazinyl group, and a triazinyl group; and

an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a benzophenanthrolinyl group, a phenazinyl group, a benzimidazolyl group, a naphthoimidazolyl group, a benzothiazolyl group, an isobenzothiazolyl group, a benzoxazolyl group, an isobenzoxazolyl group, a triazolyl group, a tetrazolyl group, an oxadiazolyl group, an imidazopyridinyl group, an imidazopyrimidinyl group, an imidazoquinolinyl group, an imidazoisoquinolinyl group, an azadibenzofuranyl group, a diazadibenzofuranyl group, an azadibenzothiophenyl group, a diazadibenzothiophenyl group, an azafluorenyl group, a diazafluorenyl group, an azaspirobifluorenyl group, and a diazaspirobifluorenyl group, each substituted with at least one selected from a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a triphenylenyl group, a fluoranthenyl group, a pyrenyl group, a chrysenyl group, a picenyl group, a perylenyl group, a benzosilolyl group, a dibenzofuranyl group, a dibenzothiophenyl group, a pyridinyl group, a pyrazinyl group, a pyrimidinyl group, a pyridazinyl group, and a triazinyl group, which are each substituted with at least one selected from a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a triphenylenyl group, a fluoranthenyl group, a pyrenyl group, a chrysenyl group, a picenyl group, a perylenyl group, a benzosilolyl group, a dibenzofuranyl group, a dibenzothiophenyl group, a pyridinyl group, a pyrazinyl group, a pyrimidinyl group, a pyridazinyl group, and a triazinyl group.

In one or more embodiments, R_{ET} may be selected from groups represented by Formulae 2-1 to 2-3:

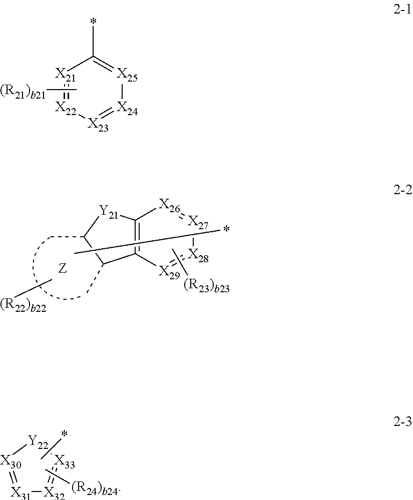

In Formulae 2-1 to 2-3, X₂₁ to X₃₃ may each independently be N or C, wherein at least one selected from X₂₁ to X₂₅ may be N, at least one selected from X₂₆ to X₂₉ may be N, and at least one selected from X₃₀ to X₃₃ may be N.

In Formulae 2-2 to 2-3, Y21 and Y22 may each independently be selected from O, S, N(R₂₅), and C(R₂₅)(R₂₆).

In an embodiment, Y21 and Y22 may each independently be selected from O, S, and N(R₂₅).

In Formulae 2-1 to 2-3, Z may be selected from a substituted or unsubstituted C₅-C₃₀ carbocyclic group and a substituted or unsubstituted C₂-C₃₀ heterocyclic group.

In an embodiment, Z may be a substituted or unsubstituted 6-membered carbocyclic group or a substituted or unsubstituted 6-membered heterocyclic group.

In Formulae 2-1 to 2-3, R₂₁ to R₂₆ may each independently be selected from hydrogen, deuterium, —F, —CI, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted alkoxy group, a substituted or unsubstituted C₃-C₁₀ cycloalkyl group, a substituted or unsubstituted heterocycloalkyl group, a substituted or unsubstituted C₃-C₁₀ cycloalkenyl group, a substituted or unsubstituted heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group, and

two neighboring substituents of R₂₁ to R₂₆ may optionally be linked to form a saturated ring or an unsaturated ring.

In addition, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group of R₂₁ to R₂₆ may be substituted with at least one selected from the substituents described above. In this case, the substituent, and the C₁-C₆₀ alkyl group, the C₂-C₆₀ alkenyl group, the C₂-C₆₀ alkynyl group, the C₁-C₆₀ alkoxy group, the C₃-C₁₀ cycloalkyl group, the C₁-C₁₀ heterocycloalkyl group, the C₃-C₁₀ cycloalkenyl group, the C₁-C₁₀ heterocycloalkenyl group, the C₆-C₆₀ aryl group, the C₆-C₆₀ aryloxy group, the C₆-C₆₀ arylthio group, the C₁-C₆₀ heteroaryl group, the monovalent non-aromatic condensed polycyclic group, and the monovalent non-aromatic condensed heteropolycyclic group may be linked via a linking group such as an alkylene group, an oxy group (—O—) or a thio group (—S—).

In Formulae 2-1 to 2-3, b21 to b23 may each independently be an integer from 1 to 4, and b24 may be an integer from 1 to 3, and

* indicates a binding site to a neighboring atom.

In an embodiment, R_{ET} may be selected from groups represented by Formulae 3-1 to 3-18:

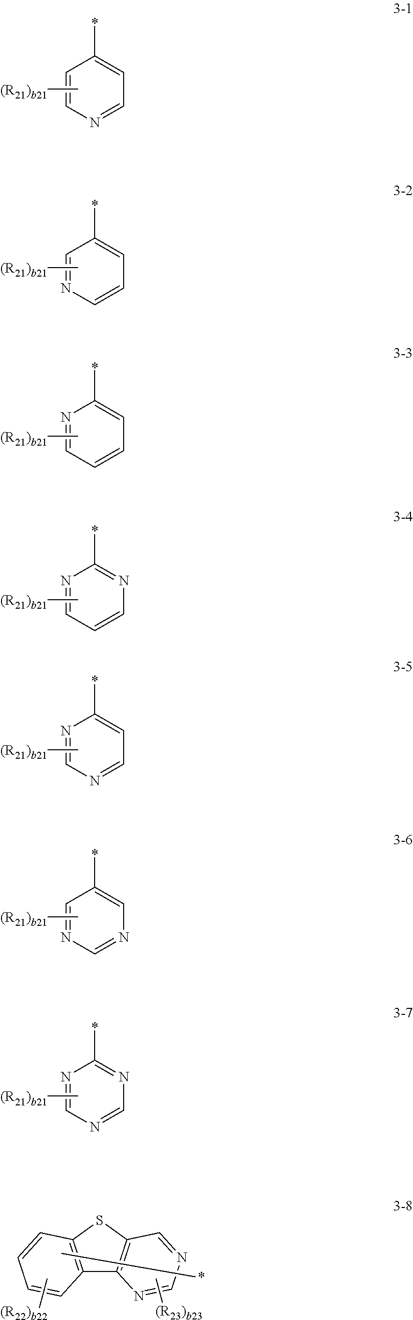

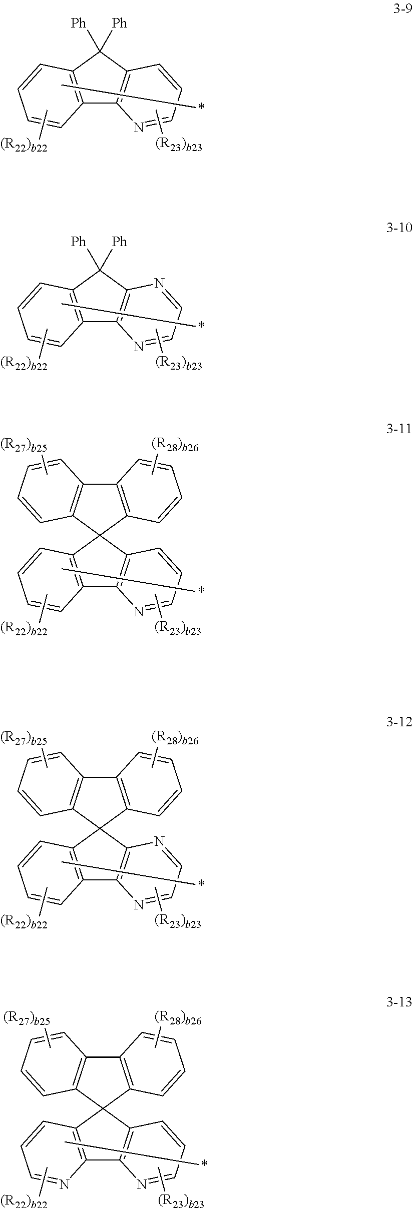

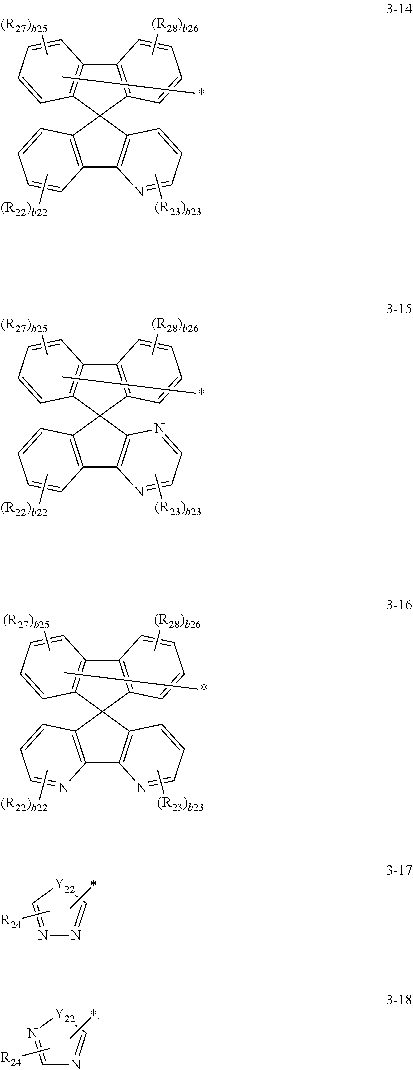

In Formulae 3-1 to 3-18,

R₂₁ to R_{24,} b21 to b23, and Y₂₂ may each independently be the same as described in Formulae 2-1 to 2-3,

R₂₇ and R₂₈ may each independently be the same as described in connection with R₂₁,

b25 and b26 may each independently be an integer from 1 to 4,

“Ph” indicates a phenyl group, and * indicates a binding site to a neighboring atom.

In an embodiment, R_{ET} may be selected from groups represented by Formulae 3-4 to 3-7.

In an embodiment, in Formulae 2-1 to 2-3, R₂₁ to R₂₆ may each independently be selected from hydrogen, deuterium, and Formulae R-1 to R-28:

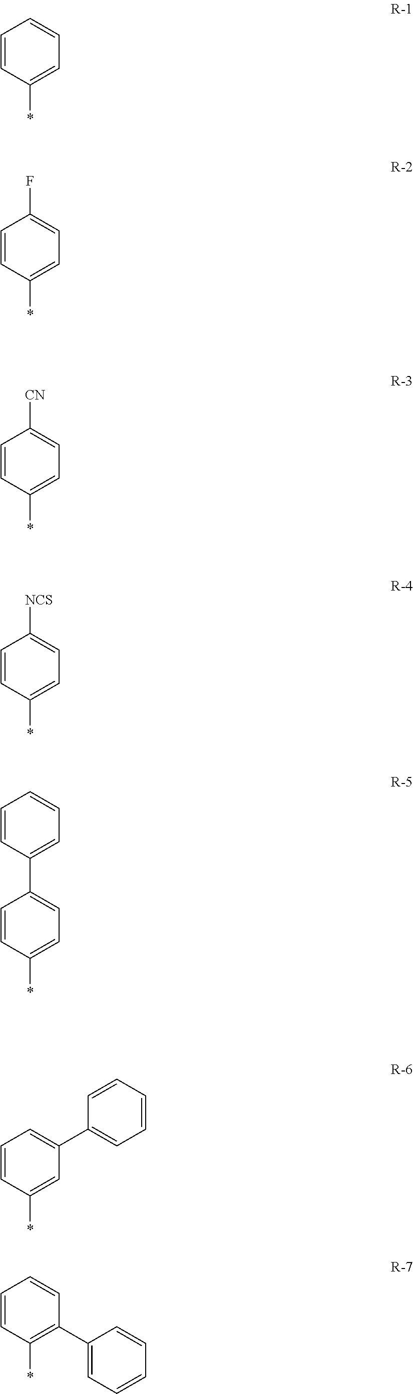

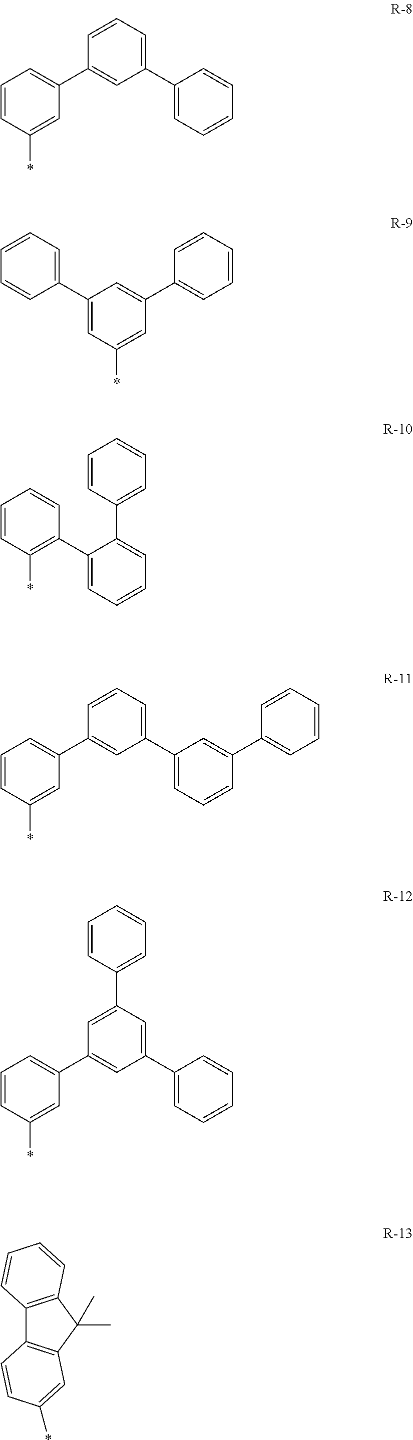 [SMILES: *c1cccc(-c2cccc(-c3ccccc3)c2)c1 ; *c1cc(-c2ccccc2)cc(-c2ccccc2)c1 ; *c1ccccc1-c1ccccc1-c1ccccc1 ; *c1cccc(-c2cccc(-c3cccc(-c4ccccc4)c3)c2)c1 ; *c1cccc(-c2cc(-c3ccccc3)cc(-c3ccccc3)c2)c1 ; *c1ccc2c(c1)C(C)(C)c1ccccc1-2]

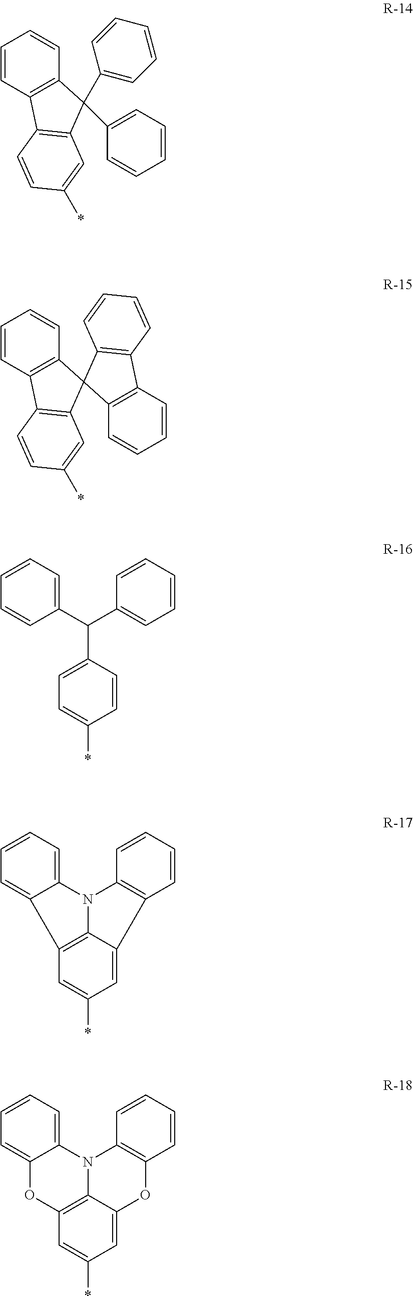 [SMILES: *c1ccc2c(c1)C(c1ccccc1)(c1ccccc1)c1ccccc1-2 ; *c1ccc2c(c1)C1(c3ccccc3-c3ccccc31)c1ccccc1-2 ; *c1ccc(C(c2ccccc2)c2ccccc2)cc1 ; *c1cc2c3ccccc3n3c4ccccc4c(c1)c23 ; *c1cc2c3c(c1)Oc1ccccc1N3c1ccccc1O2]

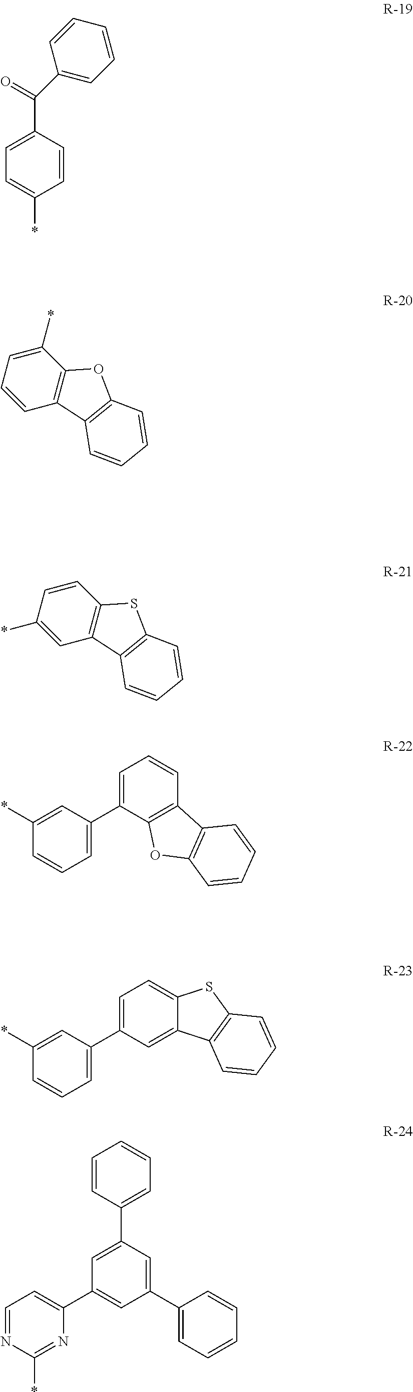 [SMILES: *c1ccc(C(=O)c2ccccc2)cc1 ; *c1cccc2c1oc1ccccc12 ; *c1ccc2sc3ccccc3c2c1 ; *c1cccc(-c2cccc3c2oc2ccccc23)c1 ; *c1cccc(-c2ccc3sc4ccccc4c3c2)c1 ; *c1nccc(-c2cc(-c3ccccc3)cc(-c3ccccc3)c2)n1]

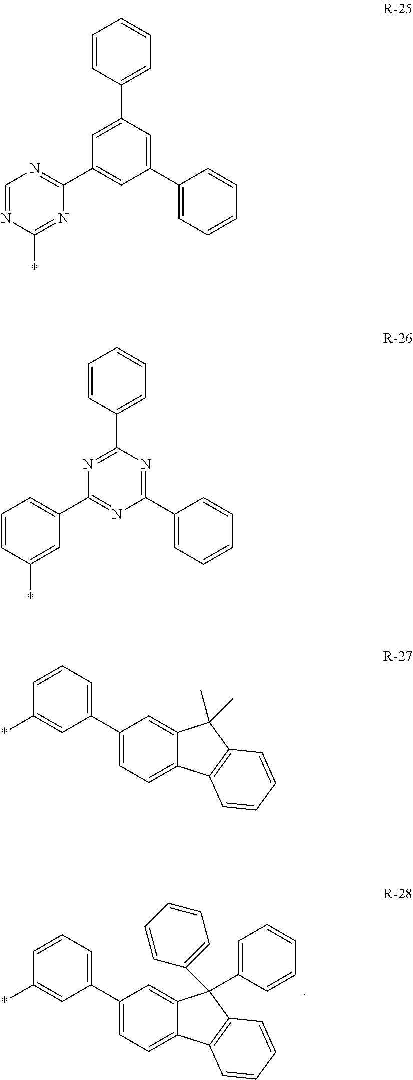 [SMILES: *c1ncnc(-c2cc(-c3ccccc3)cc(-c3ccccc3)c2)n1 ; *c1cccc(-c2nc(-c3ccccc3)nc(-c3ccccc3)n2)c1 ; *c1cccc(-c2ccc3c(c2)C(C)(C)c2ccccc2-3)c1 ; *c1cccc(-c2ccc3c(c2)C(c2ccccc2)(c2ccccc2)c2ccccc2-3)c1]

In Formulae R-1 to R-28,

* indicates a binding site to a neighboring atom.

In one or more embodiments, in Formulae 2-1 to 2-3, R₂₁ to R₂₆ may each independently be selected from hydrogen, deuterium, and Formulae R-1, R-9, R-12 to R-15, and R-20 to R-28.

In Formulae 1-1 and 1-2, R₁ to R₅ may each independently be selected from hydrogen, deuterium, —F, —CI, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), —B(Q₆)(Q₇), —P(═O)(Q₈)(Q₉), and —C(═O)(Q₁₀).

In an embodiment, in Formulae 1-1 and 1-2, R₁ to R₅ may each independently be selected from:

hydrogen, deuterium, a methyl group, an ethyl group, an n-propyl group, an iso-propyl group, an n-butyl group, an iso-butyl group, a sec-butyl group, a tert-butyl group, an n-pentyl group, an iso-pentyl group, a tert-pentyl group, a neo-pentyl group, a 1,2-dimethylpropyl group, an n-hexyl group, an iso-hexyl group, a 1,3-dimethylbutyl group, a 1-iso-propylpropyl group, a 1,2-dimethylbutyl group, an n-heptyl group, a 1,4-dimethylpentyl group, a 3-ethylpentyl group, a 2-methyl-1-iso-propylpropyl group, a 1-ethyl-3-methylbutyl group, an n-octyl group, a 2-ethylhexyl group, a 3-methyl-1-iso-propylbutyl group, a 2-methyl-1- iso-propyl group, a 1-tert-butyl-2-methylpropyl group, an n-nonyl group, a 3,5,5-trimethyldecyl group, an n-decyl group, an isodecyl group, an n-undecyl group, a 1-methyldecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, an n-eicosyl group, an n-heneicosyl group, an n-docosyl group, an n-tricosyl group, an n-tetracosyl group, a methoxy group, an ethoxy group, a propoxy group, an iso-propoxy group, an n-butoxy group, an iso-butoxy group, a sec-butoxy group, a tert-butoxy group, an n-pentoxy group, an iso-pentoxy group, a tert-pentoxy group, a neo-pentoxy group, an n-hexyloxy group, an iso-hexyloxy group, a heptyloxy group, an octyloxy group, a nonyloxy group, a decyloxy group, an undeoxy group, a dodecyloxy group, a tridecyloxy group, a tetradecyloxy group, a pentadecyloxy group, a hexadecyloxy group, a heptadecyloxy group, an octadecyloxy group, a 2-ethylhexyloxy group, a 3-ethylpentyloxy group, a phenoxy group, a 1-naphthyloxy group, a 2-naphthyloxy group, a 2-azulenyloxy group, a 2-furanyloxy group, a 2-thienyloxy group, a 2-indoleyloxy group, a 3-indoleyloxy group, a 2-benzofuriloxy group, a 2-benzothienyloxy group, an N-methylamino group, an N-ethylamino group, an N-propylamino group, an N-iso-propylamino group, an N-butylamino group, an N-iso-butylamino group, an N-sec-butylamino group, an N-tert-butylamino group, an N-pentylamino group, an N-hexylamino group, an N,N-dimethylamino group, an N,N-diethylamino group, an N,N-dipropylamino group, an N,N-di-iso-propylamino group, an N,N-dibutylamino group, an N,N-di-iso-butylamino group, an N,N-dipentylamino group, and an N,N-dihexylamino group;

a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group; and

a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

In Formulae 1-1 and 1-2, b1 and b2 may each independently be an integer from 0 to 4, and b3 and b4 may each independently be an integer from 0 to 3.

In Formulae 1-1 and 1-2, n1 may be an integer from 1 to 8.

In an embodiment, the heterocyclic compound may be selected from the following compounds, but embodiments of the present disclosure are not limited thereto:

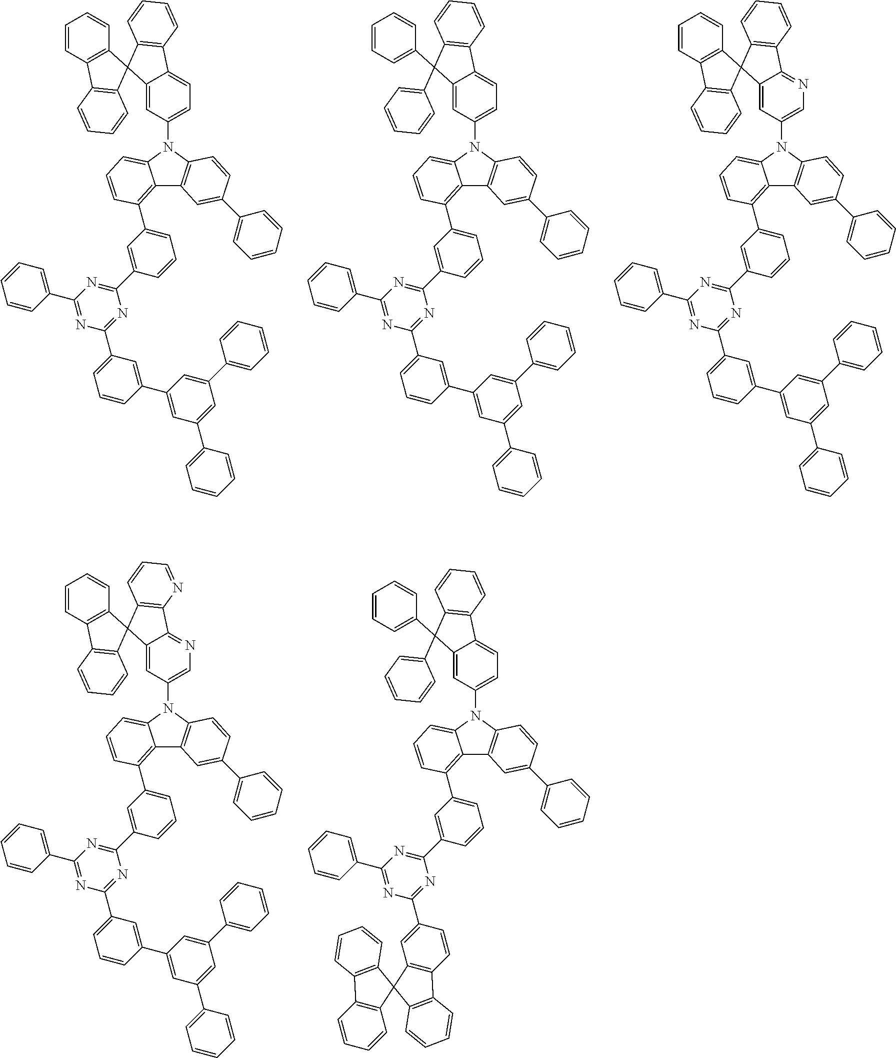

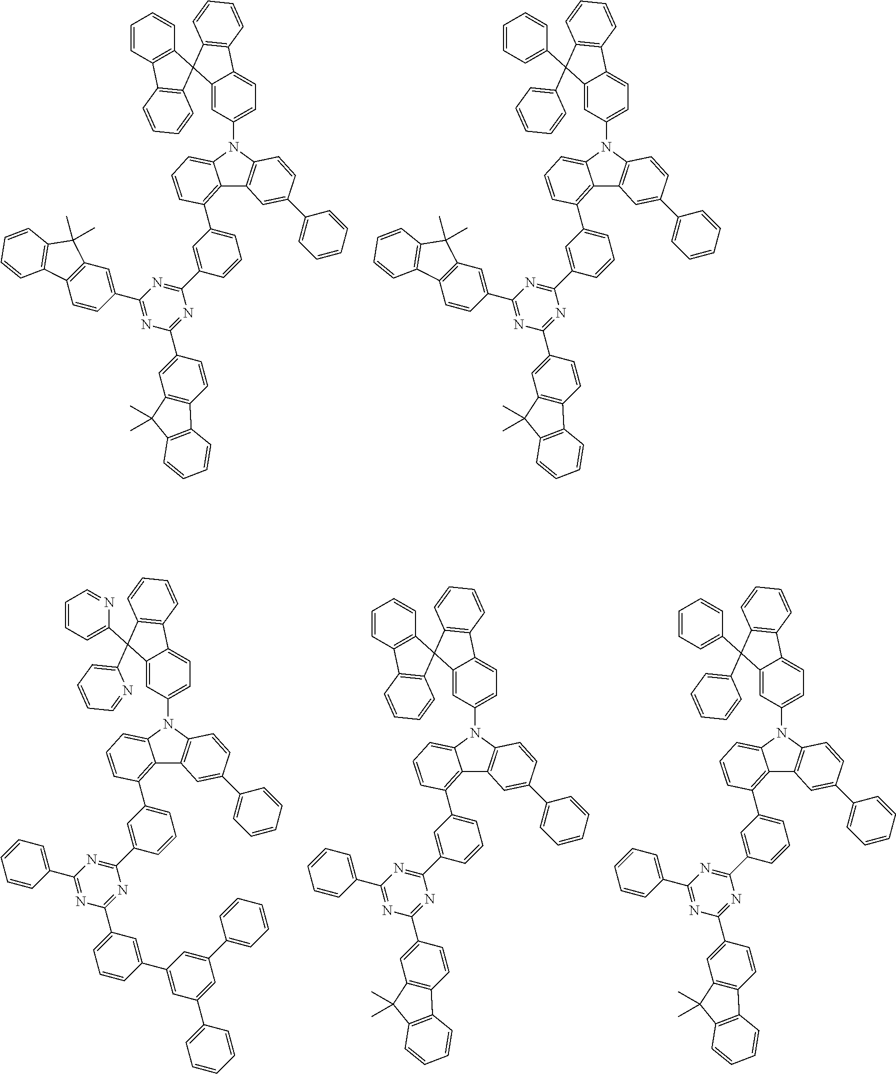

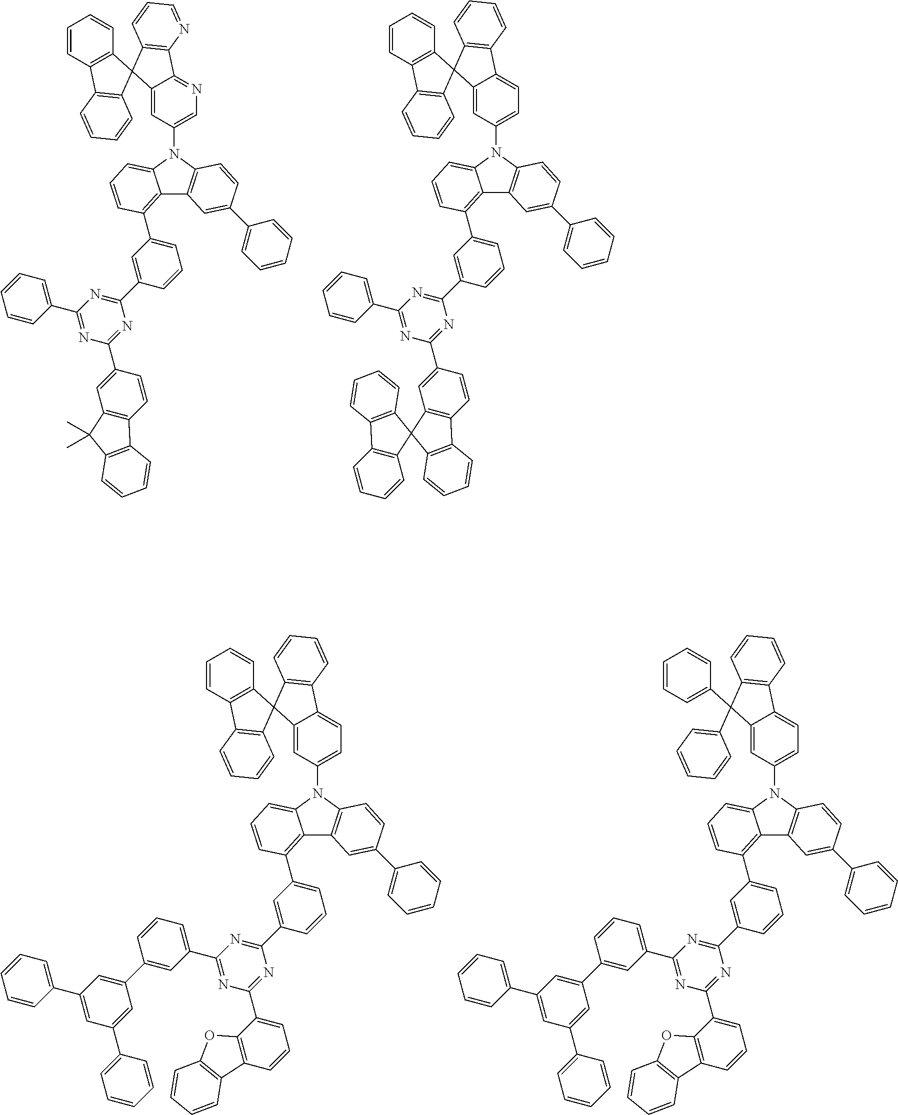

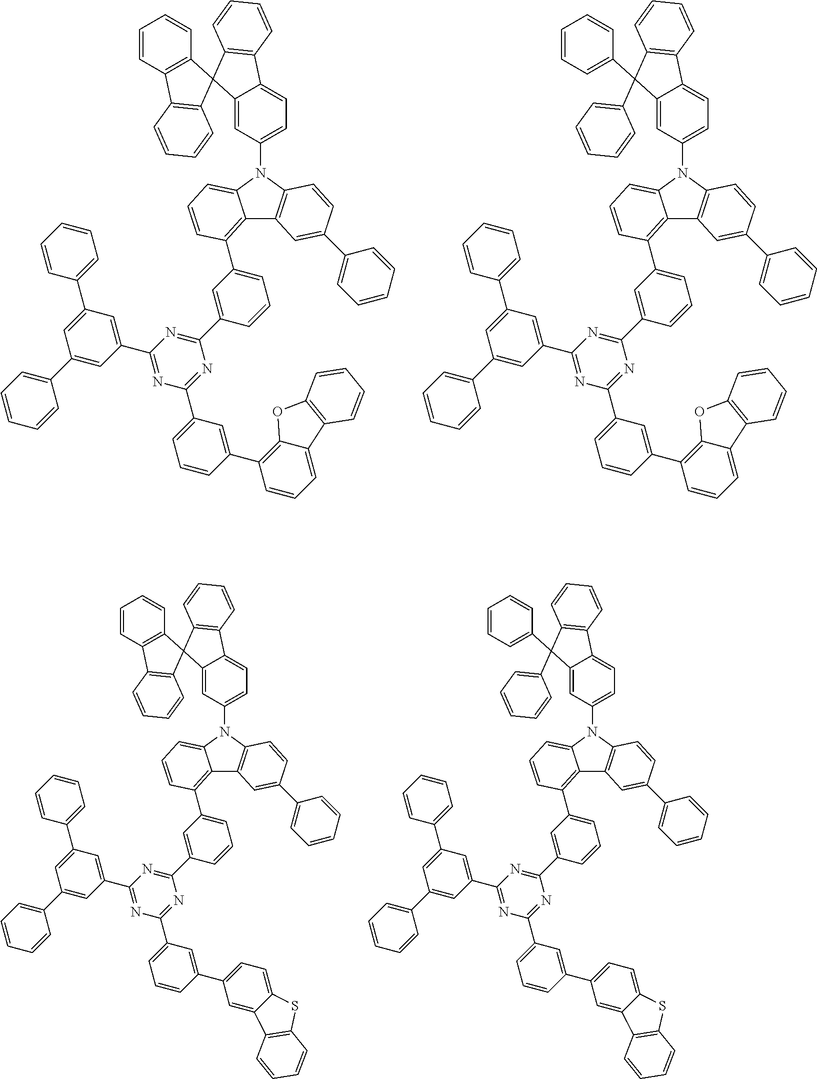

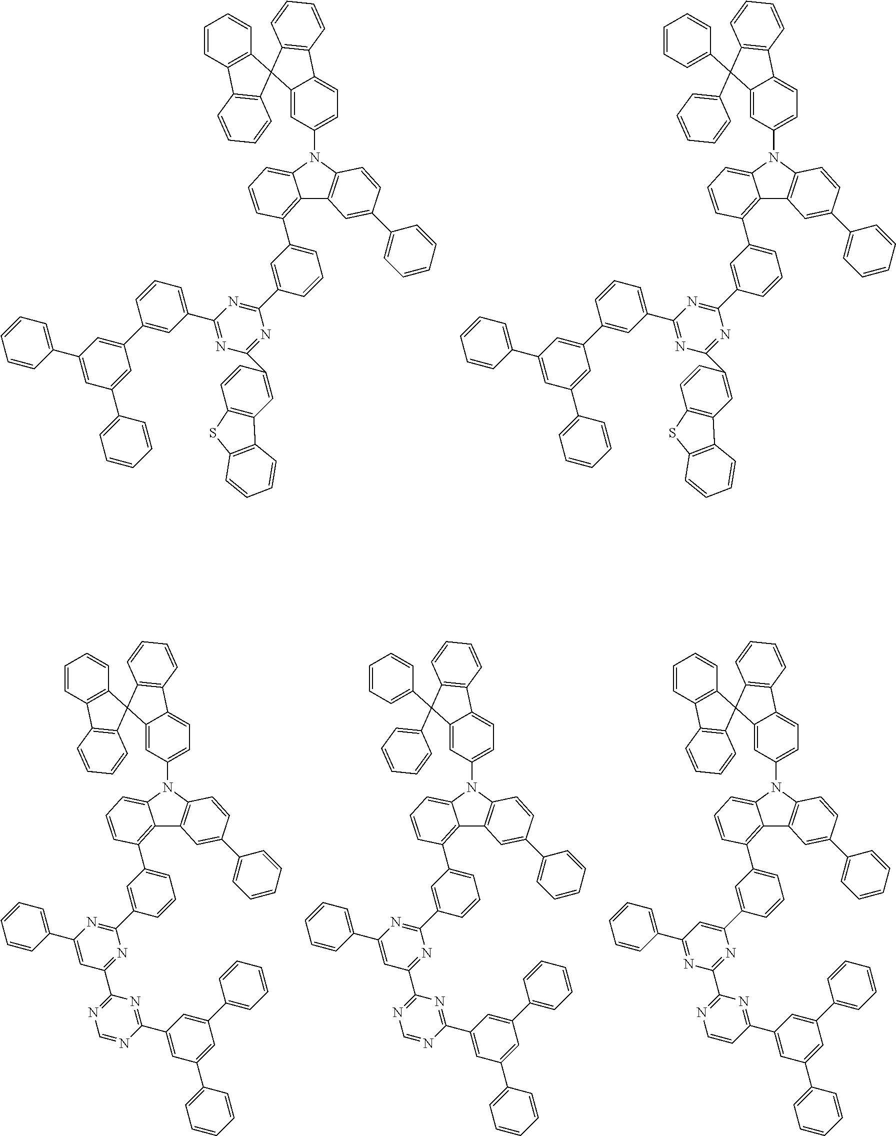

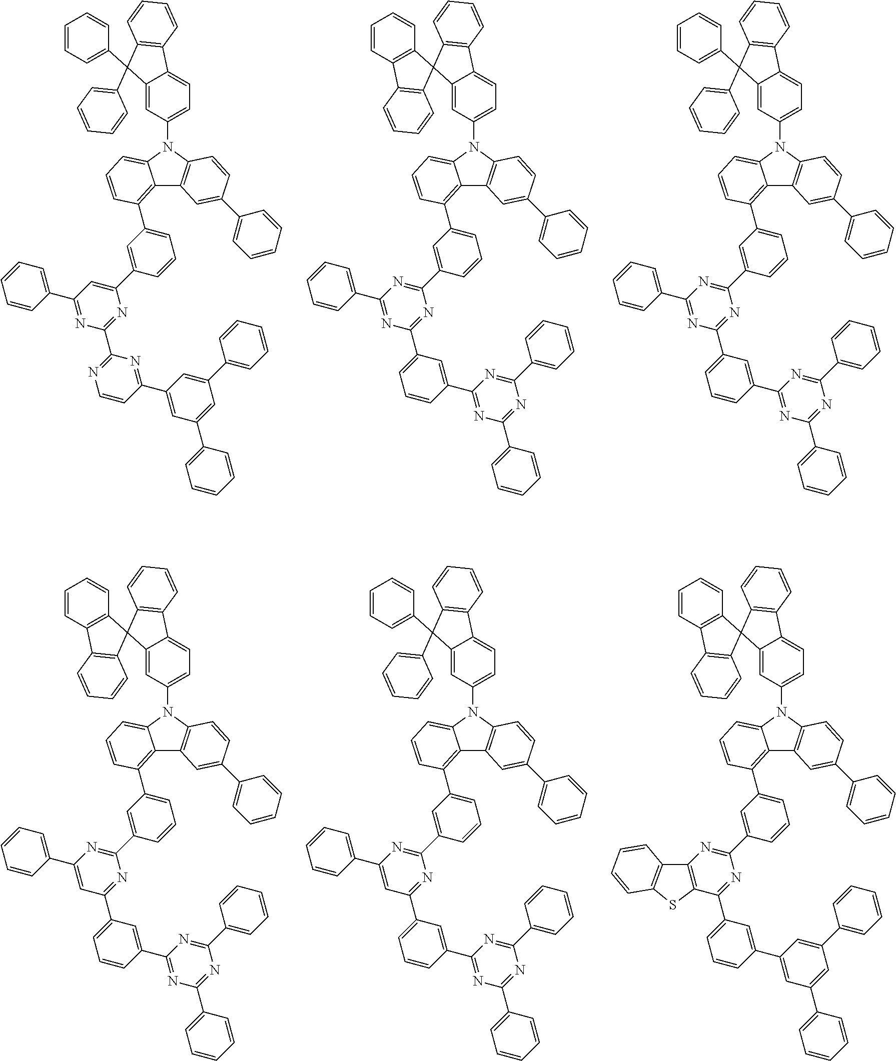

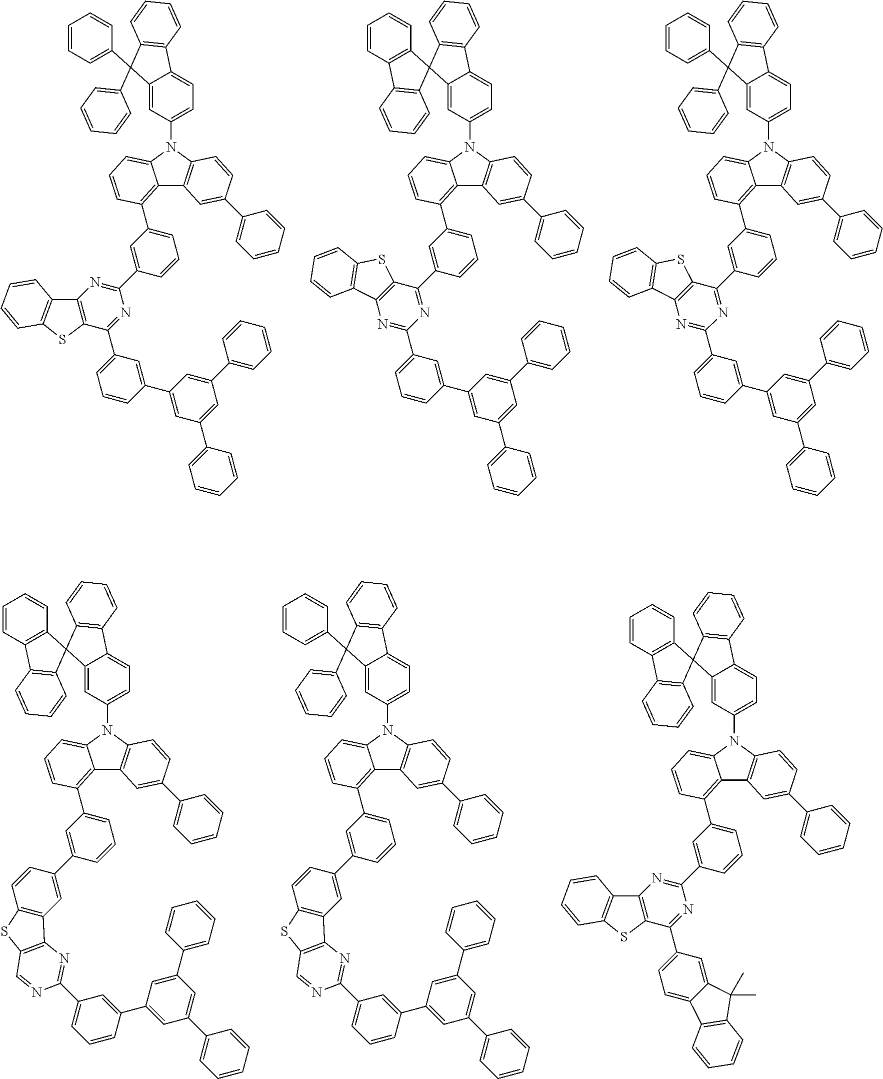

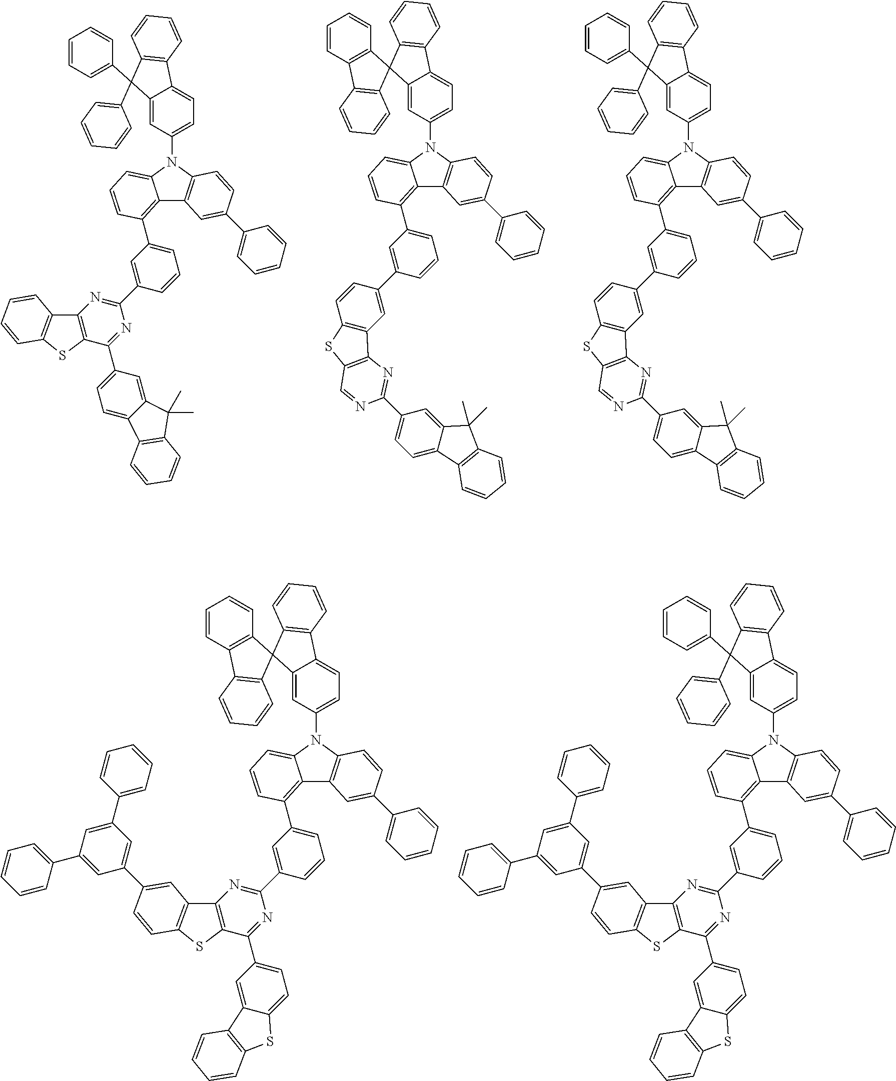

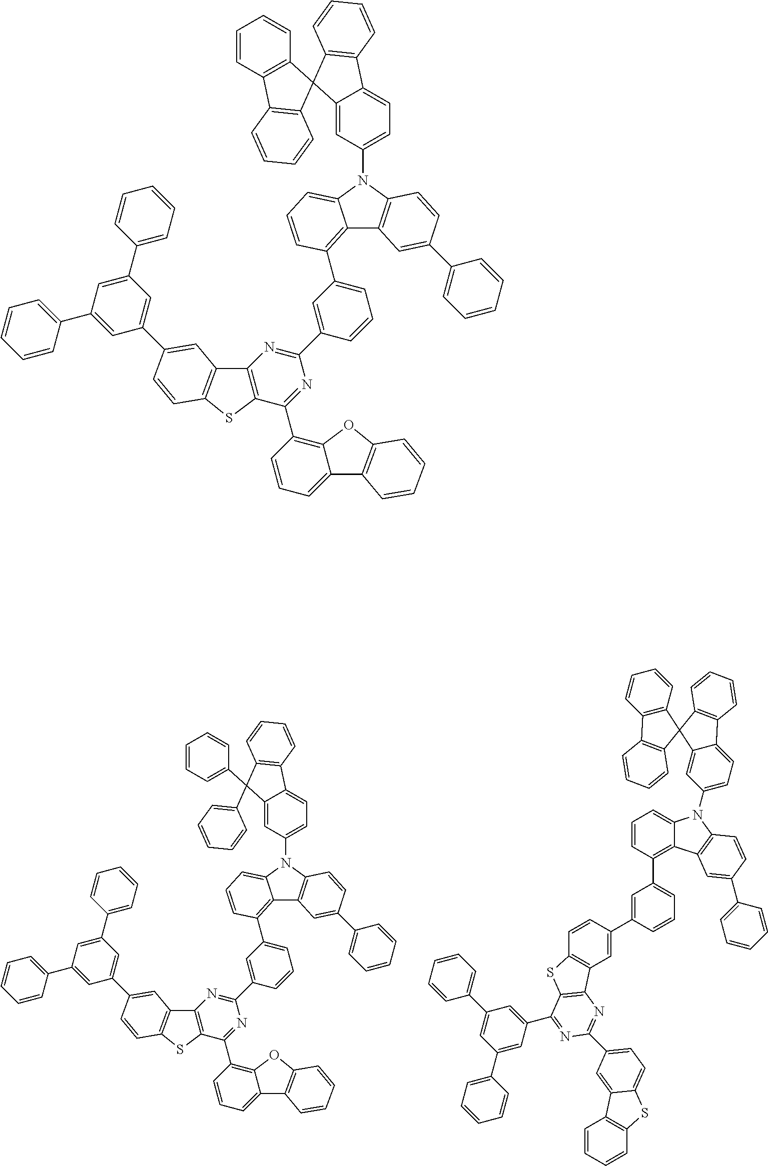 [SMILES: c1ccc(-c2cc(-c3ccccc3)cc(-c3ccc4sc5c(-c6cccc7c6oc6ccccc67)nc(-c6cccc(-c7cccc8c7c7cc(-c9ccccc9)ccc7n8-c7ccc8c(c7)C7(c9ccccc9-c9ccccc97)c7ccccc7-8)c6)nc5c4c3)c2)cc1 ; c1ccc(-c2cc(-c3ccccc3)cc(-c3ccc4sc5c(-c6cccc7c6oc6ccccc67)nc(-c6cccc(-c7cccc8c7c7cc(-c9ccccc9)ccc7n8-c7ccc8c(c7)C(c7ccccc7)(c7ccccc7)c7ccccc7-8)c6)nc5c4c3)c2)cc1 ; c1ccc(-c2cc(-c3ccccc3)cc(-c3nc(-c4ccc5sc6ccccc6c5c4)nc4c3sc3ccc(-c5cccc(-c6cccc7c6c6cc(-c8ccccc8)ccc6n7-c6ccc7c(c6)C6(c8ccccc8-c8ccccc86)c6ccccc6-7)c5)cc34)c2)cc1]

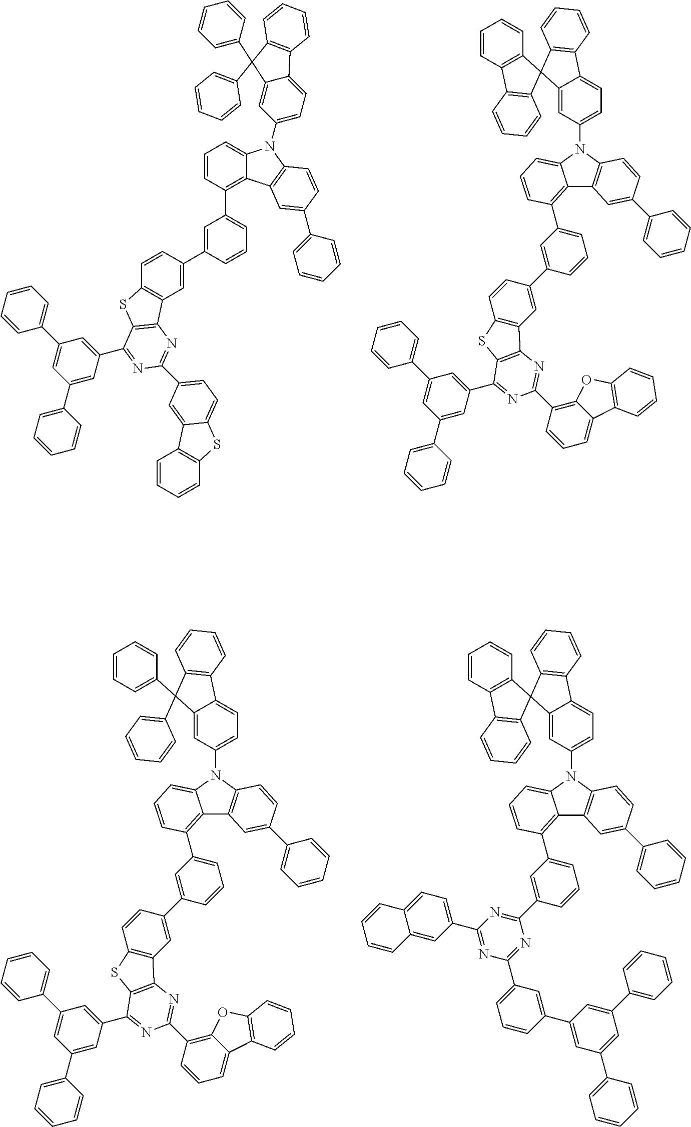

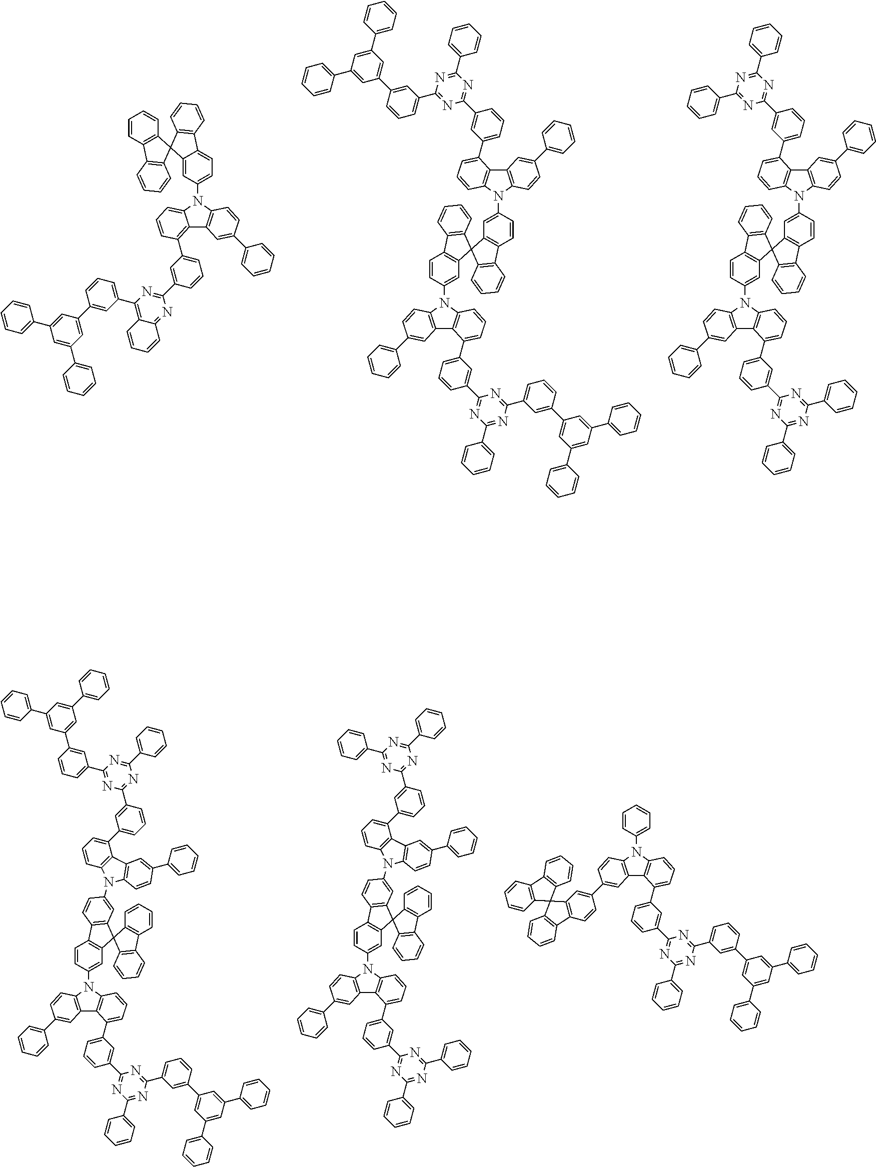

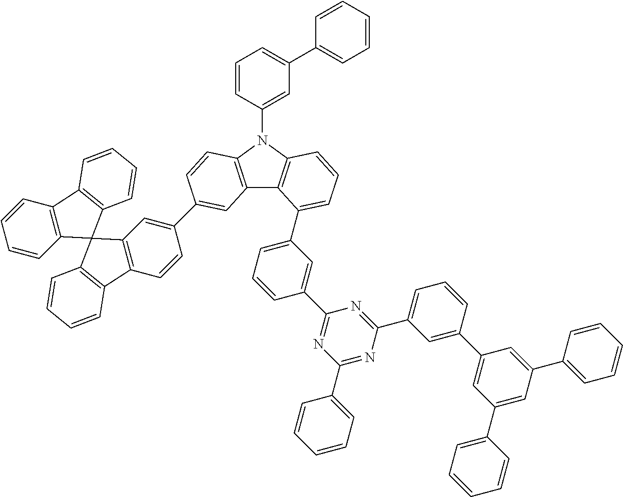 [SMILES: c1ccc(-c2cc(-c3ccccc3)cc(-c3cccc(-c4nc(-c5ccccc5)nc(-c5cccc(-c6cccc7c6c6cc(-c8ccc9c(c8)C8(c%10ccccc%10-c%10ccccc%108)c8ccccc8-9)ccc6n7-c6cccc(-c7ccccc7)c6)c5)n4)c3)c2)cc1]

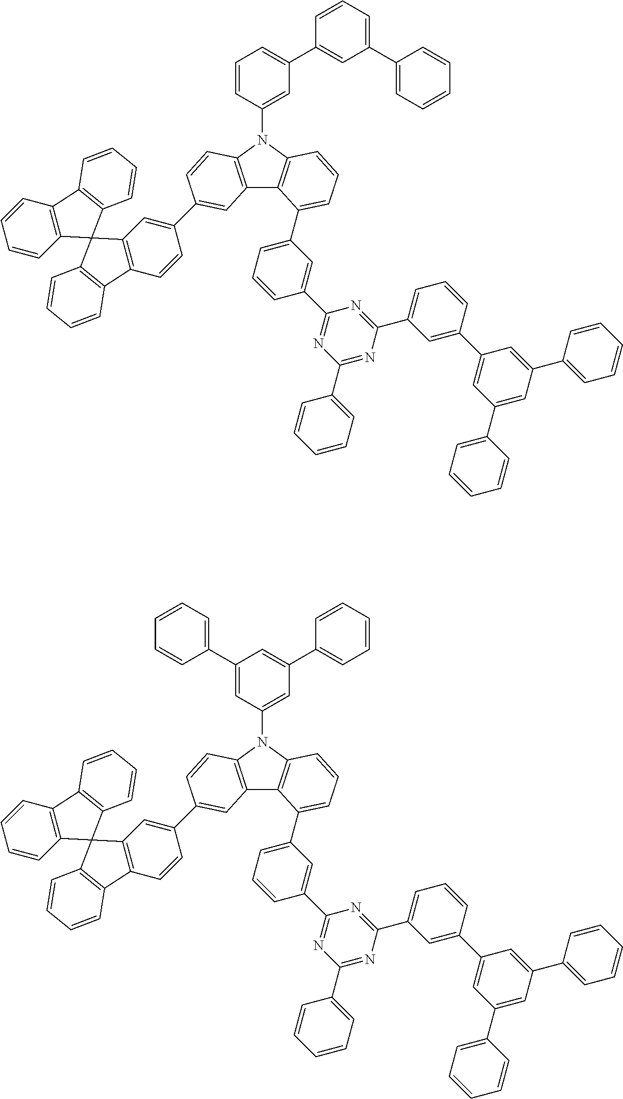 [SMILES: c1ccc(-c2cccc(-c3cccc(-n4c5ccc(-c6ccc7c(c6)C6(c8ccccc8-c8ccccc86)c6ccccc6-7)cc5c5c(-c6cccc(-c7nc(-c8ccccc8)nc(-c8cccc(-c9cc(-c%10ccccc%10)cc(-c%10ccccc%10)c9)c8)n7)c6)cccc54)c3)c2)cc1 ; c1ccc(-c2cc(-c3ccccc3)cc(-c3cccc(-c4nc(-c5ccccc5)nc(-c5cccc(-c6cccc7c6c6cc(-c8ccc9c(c8)C8(c%10ccccc%10-c%10ccccc%108)c8ccccc8-9)ccc6n7-c6cc(-c7ccccc7)cc(-c7ccccc7)c6)c5)n4)c3)c2)cc1]

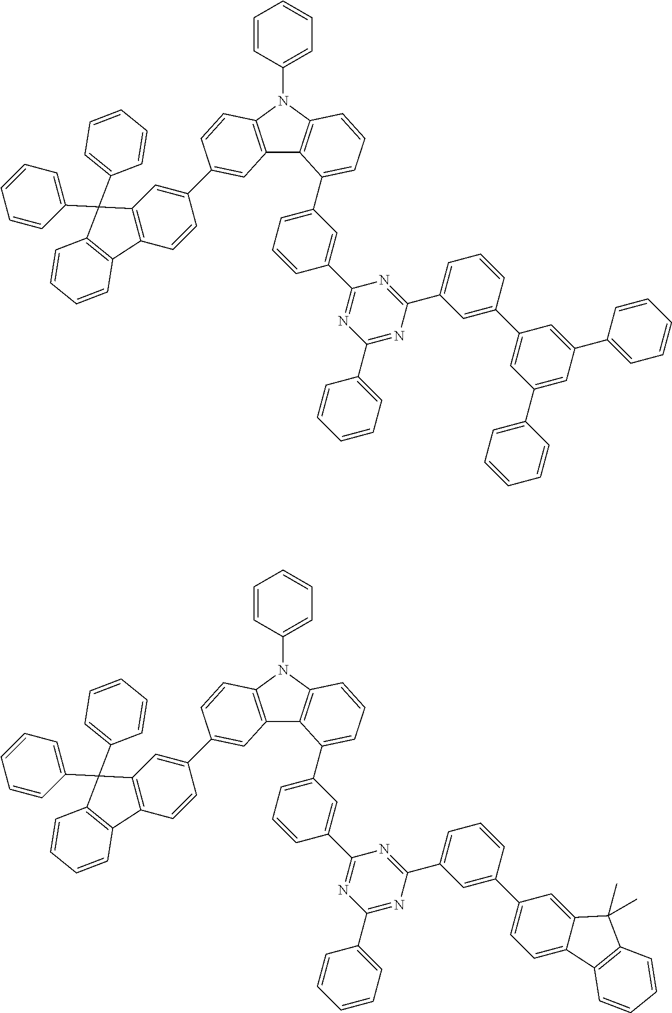 [SMILES: c1ccc(-c2cc(-c3ccccc3)cc(-c3cccc(-c4nc(-c5ccccc5)nc(-c5cccc(-c6cccc7c6c6cc(-c8ccc9c(c8)C(c8ccccc8)(c8ccccc8)c8ccccc8-9)ccc6n7-c6ccccc6)c5)n4)c3)c2)cc1 ; CC1(C)c2ccccc2-c2ccc(-c3cccc(-c4nc(-c5ccccc5)nc(-c5cccc(-c6cccc7c6c6cc(-c8ccc9c(c8)C(c8ccccc8)(c8ccccc8)c8ccccc8-9)ccc6n7-c6ccccc6)c5)n4)c3)cc21]

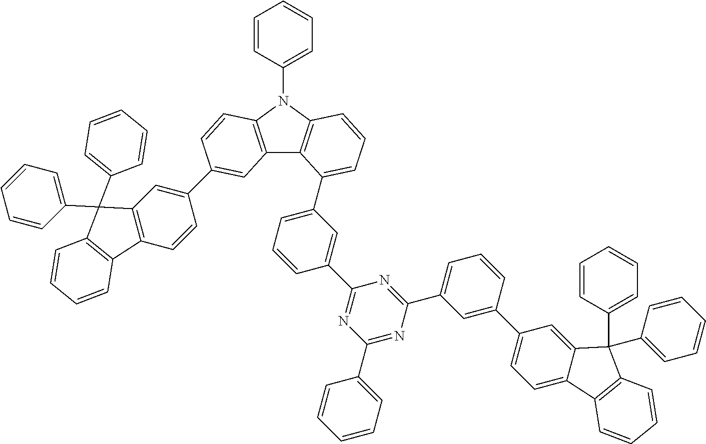 [SMILES: c1ccc(-c2nc(-c3cccc(-c4ccc5c(c4)C(c4ccccc4)(c4ccccc4)c4ccccc4-5)c3)nc(-c3cccc(-c4cccc5c4c4cc(-c6ccc7c(c6)C(c6ccccc6)(c6ccccc6)c6ccccc6-7)ccc4n5-c4ccccc4)c3)n2)cc1]

Since the compound represented by Formula 1-1 or 1-2 includes a structure in which a carbazole ring is linked to a fluorene ring, its electron transport capability and electron injection capability may be excellent. Therefore, the heterocyclic compound represented by Formula 1 may be suitable as a material for an organic light-emitting device.

The heterocyclic compound represented by Formula 1 may exhibit high solubility to a solvent (in particular, an ester-based organic solvent). In addition, since the heterocyclic compound represented by Formula 1 has a high glass transition temperature (T_{g}), the heterocyclic compound is hardly modified or deformed by heat when the solvent is removed after a film is formed by solution coating. Therefore, the heterocyclic compound represented by Formula 1 may be applied to solution coating. In addition, an organic layer formed using the heterocyclic compound represented by Formula 1 by solution coating may have less unevenness, uniform thickness, and a smooth surface. Therefore, even when the organic light-emitting device using the heterocyclic compound represented by Formula 1 is manufactured by solution coating, a change in application of a driving voltage and a change in light emission are small, thereby improving current efficiency and an emission lifespan.

The heterocyclic compound represented by Formula 1 has a relatively high glass transition temperature and excellent thermal stability. Therefore, the organic light-emitting device including the heterocyclic compound represented by Formula 1 is relatively stable to the driving heat. Since the deterioration and deformation of the organic light-emitting device due to the driving heat are suppressed, the organic light-emitting device including the heterocyclic compound represented by Formula 1 may exhibit improved lifespan.

At this time, the heterocyclic compound represented by Formula 1 may have a glass transition temperature of about 120° C. or more, for example, about 125° C. In an embodiment, the heterocyclic compound may have a glass transition temperature of about 140° C. or more. The glass transition temperature may be measured by, for example, the method defined in JIS K 7121 or ISO 3146. In the following Examples, the glass transition temperature was measured by differential scanning calorimetry.

The heterocyclic compound represented by Formula 1 has a molecular weight of about 850 grams per mole (g/mol) to about 3,000 g/mol, for example, about 850 g/mol to about 2,500 g/mol. While not wishing to be bound by theory, it is understood that when the molecular weight of the heterocyclic compound is 850 g/mol or more, the heat resistance of the heterocyclic compound may be improved. While not wishing to be bound by theory, it is understood that when the molecular weight of the heterocyclic compound is 3,000 g/mol or less, the solubility of the heterocyclic compound to the solvent (for example, the ester-based organic solvent) may be improved.

In addition, the solubility of the heterocyclic compound to methyl benzoate at a temperature of 25° C. may be 0.5 percent by weight (weight %) or more, for example, 1 weight % or more.

The heterocyclic compound represented by Formula 1 may be included in an organic layer disposed between a pair of electrodes of an organic light-emitting device. For example, the heterocyclic compound represented by Formula 1 may be included in an emission layer, and may be used as a host.

The heterocyclic compound represented by Formula 1 may be synthesized by using a known organic synthesis method. A method of synthesizing the heterocyclic compound represented by Formula 1 can be understood by those of ordinary skill in the art by referring to Examples provided below.

Material for Light-Emitting Device

A material for a light-emitting device according to an embodiment may include the heterocyclic compound represented by Formula 1. Since the heterocyclic compound represented by Formula 1 has high solubility to the solvent and excellent thermal stability as described above, a thin film having high quality may be manufactured even by wet coating. In addition, the heterocyclic compound represented by Formula 1 may have high charge transport capability. Therefore, the material for the organic light-emitting device including the heterocyclic compound represented by Formula 1 may be used for, for example, the charge transport layer and the emission layer of the organic light-emitting device.

Even when the organic light-emitting device using the heterocyclic compound represented by Formula 1 is manufactured by using wet coating, the current efficiency and the emission lifespan may be improved.

Therefore, the material for the light-emitting device including the heterocyclic compound represented by Formula 1 may be used to manufacture the light-emitting device. In this case, the light-emitting device having excellent current efficiency and emission lifespan may be manufactured by using a coating process.

Composition

Hereinafter, a composition according to an embodiment will be described in detail.

The composition is used to form, for example, each layer of a light-emitting device by using solution coating.

The composition may include the heterocyclic compound represented by Formula

In an embodiment, the composition may further include a light-emitting material.

The light-emitting material is not particularly limited as long as the light-emitting material has a light-emitting function. The light-emitting material may be a fluorescent dopant, a phosphorescent dopant, a quantum dot, or the like.

The fluorescent dopant is a compound that can emit light from singlet exciton. For example, the fluorescent dopant may be a perylene and a derivative thereof, a rubrene and a derivative thereof, a coumarin and a derivative thereof, or a 4-dicyanomethylene-2-(p-dimethylaminostyryl)-6-methyl-4H-pyran (DCM) and a derivative thereof, but embodiments of the present disclosure are not limited thereto.

The phosphorescent dopant is a compound that can emit light from triplet exciton, and may be an organometallic compound. For example, the phosphorescent dopant may be an iridium complex, such as bis[2-(4,6-difluorophenyl)pyridinate] picolinate iridium(III) (Flrpic), bis(1-phenylisoquinoline)(acetylacetonate) iridium(III) (Ir(piq)₂(acac)), tris(2-phenylpyridine) iridium(III) (Ir(ppy)₃), or tris(2-(3-p-xylyl)phenyl)pyridine iridium(III), an osmium complex, a platinum complex, or the like, but embodiments of the present disclosure are not limited thereto.

The quantum dot may be a nanoparticle including group II-VI semiconductor, group III-V semiconductor, or group IV-IV semiconductor. For example, the quantum dot may be CdO, CdS, CdSe, CdTe, ZnO, ZnS, ZnSe, ZnTe, HgS, HgSe, HgTe, MgSe, MgS CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, GaN, GaP, GaAs, AlN, AlP, AlAs, InN, InP, InAs, InSb, GaNP, GaNAs, GaNSb, GaPAs, GaPSb, AlNP, AlNAs, AlNSb, AlPAs, AlPSb, InNP, InNAs, InPAs, InPSb, GaAlNP, SnS, SnSe, SnTe, PbS, PbSe, PbTe, SnSeS, SnSeTe, SnSTe, PbSeS, PbSeTe, PbSTe, SnPbS, SnPbSe, SnPbTe, or the like, but embodiments of the present disclosure are not limited thereto. In addition, the diameter of the quantum dot is not particularly limited, but may be in a range of about 1 nanometer (nm) to about 20 nm. The quantum dot may be a single core structure, or may be a core-shell structure.

The composition may further include at least one selected from a compound represented by Formula 4-1, a compound represented by Formula 4-2, a compound represented by Formula 5, a compound represented by Formula 6, and a compound represented by Formula 7:

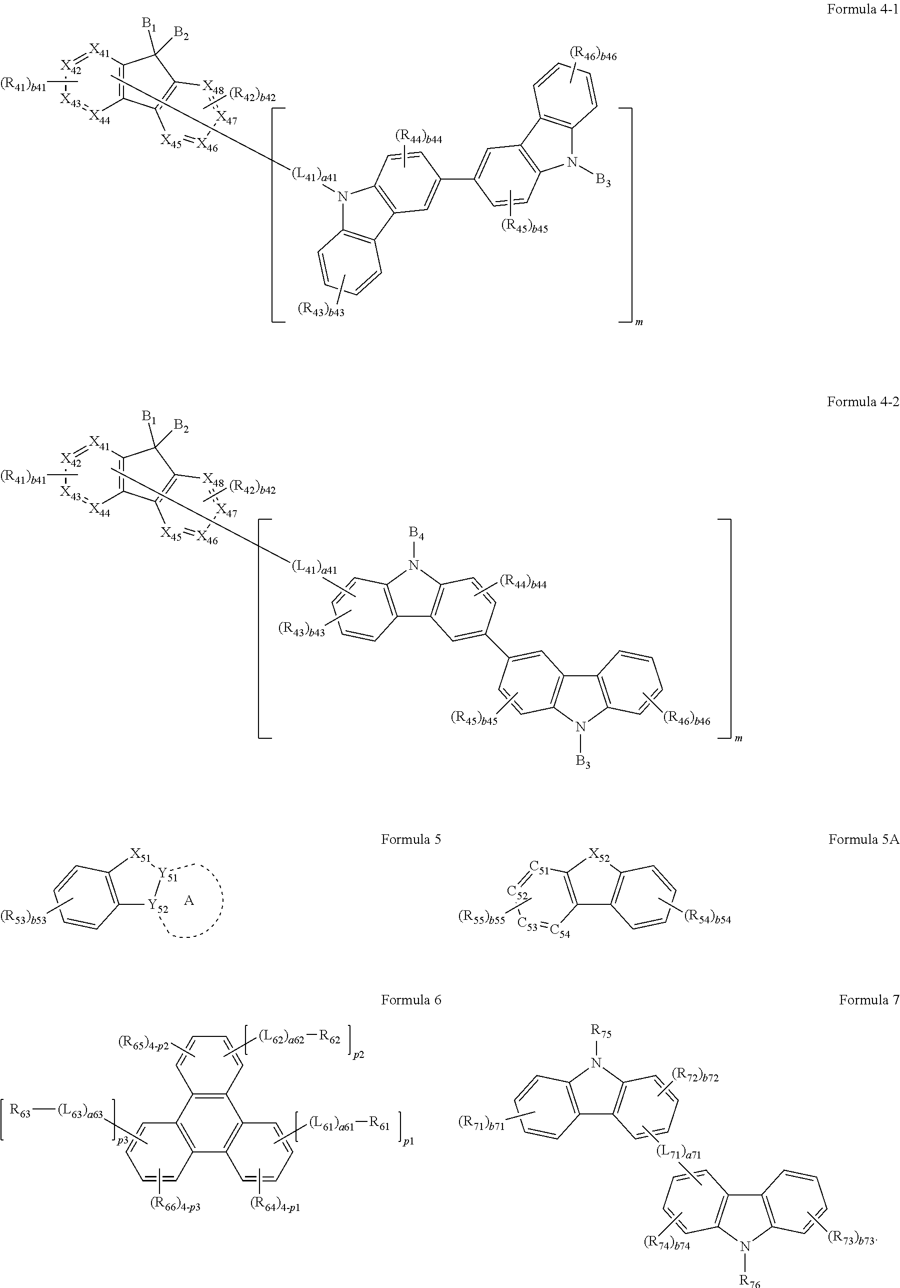

In Formulae 4-1, 4-2, 5, 5A, 6, and 7,

X₄₁ to X₄₈ may each independently be N or C,

at least one selected from X₄₁ to X₄₈ may be carbon linked to —(L₄₁)ₐ₄₁—,

X₅₁ may be N(R₅₁), O, or S, and X₅₂ may be N(R₅₂), O, or S,

C₅₁ to C₅₄ may be each a carbon atom (C), two neighboring groups selected from C₅₁ to C₅₄ may be condensed with a group represented by Formula 5 via Y₅₁ and Y₅₂,

L₄₁, L₆₁ to L₆₃, and L₇₁ may each independently be a substituted or unsubstituted C₅-C₃₀ carbocyclic group and a substituted or unsubstituted C₁-C₃₀ heterocyclic group,

a41, a61 to a63, and a71 may each independently be an integer from 0 to 3,

A may be a group represented by Formula 5A,

R₄₁ to R₄₆, R₅₁ to R_{55,} R₆₁ to R₆₆, and R₇₁ to R₇₆ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), and —B(Q₆)(Q₇),

b41 to b43, b46, b53 to b54, and b71 to b73 may each independently be an integer from 0 to 4,

b44, b45, b72, and b74 may each independently be an integer from 0 to 3,

b55 may be an integer from 0 to 2,

B₁ and B₄ may each independently be selected from a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted heterocycloalkyl group, a substituted or unsubstituted C₃-C₁₀ cycloalkenyl group, a substituted or unsubstituted heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group,

m may be an integer from 1 to 8, and

p1 to p3 may each independently be an integer from 0 to 4.

The composition may further include a solvent.

The solvent included in the composition is not particularly limited as long as the heterocyclic compound represented by Formula 1 is dissolved therein. Examples of the solvent may be toluene, xylene, ethylbenzene, diethylbenzene, mesitylene, propylbenzene, cyclohexylbenzene, dimethoxybenzene, anisole, ethoxytoluene, phenoxytoluene, iso-propylbiphenyl, dimethylanisole, phenyl acetate, phenyl propionic acid, methyl benzoate, and ethyl benzoate, but embodiments of the present disclosure are not limited thereto.

In the composition, the concentration of the heterocyclic compound represented by Formula 1 may be in a range from about 0.1 weight % to about 10 weight %, for example, about 0.5 weight % to about 5 weight %, but embodiments of the present disclosure are not limited thereto. While not wishing to be bound by theory, it is understood that when the concentration of the heterocyclic compound is within this range, coatability may be improved.

Therefore, the composition may be used as the material for the light-emitting device (for example, an organic light-emitting device, a quantum dot light-emitting device, or the like). For example, the composition may be used for the emission layer, the charge injection layer, and/or the charge transport layer of the light-emitting device. In another example, the composition may be used for the emission layer of the light-emitting device. In particular, the composition may be used to manufacture the light-emitting device by using solution coating. At this time, the current efficiency and the emission lifespan of the light-emitting device may be maintained or improved.

Organic Light-Emitting Device

Hereinafter, an organic light-emitting device according to an embodiment will be described in detail with reference to the FIGURE. The FIGURE is a schematic view of an organic light-emitting device according to an embodiment.

An organic light-emitting device 100 according to an embodiment may include a substrate 110, a first electrode 120 on the substrate 110, a hole injection layer 130 on the first electrode 120, a hole transport layer 140 on the hole injection layer 130, an emission layer 150 on the hole transport layer 140, an electron transport layer 160 on the emission layer 150, an electron injection layer 170 on the electron transport layer 160, and a second electrode 180 on the electron injection layer 170.

The heterocyclic compound represented by Formula 1 may be included in, for example, an organic layer between the first electrode 120 and the second electrode 180 (for example, the hole injection layer 130, the hole transport layer 140, the emission layer 150, the electron transport layer 160, or the electron injection layer 170). For example, the heterocyclic compound represented by Formula 1 may be included in the emission layer 150 as a host. Alternatively, the heterocyclic compound represented by Formula 1 may be included in another organic layer other than the emission layer 150. For example, the heterocyclic compound represented by Formula 1 may be included in the hole injection layer 130 and/or the hole transport layer 140 as a charge transport material.

The term “organic layer” as used herein refers to a single layer and/or a plurality of layers between the first electrode and the second electrode of the organic light-emitting device. The “organic layer” may include, in addition to an organic compound, an organometallic compound including metal.

The expression “(an organic layer) includes at least one organometallic compound” as used herein includes an embodiment in which “(an organic layer) includes identical heterocyclic compound represented by Formula 1” or an embodiment in which “(an organic layer) includes two or more different heterocyclic compounds represented by Formula 1”.

For example, the organic layer may include, as the heterocyclic compound, only Compound 1. In this embodiment, Compound 1 may be included in an emission layer of the organic light-emitting device. In one or more embodiments, the organic layer may include, as the heterocyclic compound, Compound 1 and Compound 2. In this embodiment, Compound 1 and Compound 2 may be included in an identical layer (for example, Compound 1 and Compound 2 may all be included in an emission layer).

The substrate 110 may be any substrate that is used in an organic light-emitting device according to the related art. For example, the substrate 110 may be a glass substrate, a silicon substrate, or a transparent plastic substrate, each having excellent mechanical strength, thermal stability, surface smoothness, ease of handling, and water resistance, but embodiments of the present disclosure are not limited thereto.

The first electrode 120 may be formed on the substrate 110. The first electrode 120 may be, for example, an anode, and may be formed of a material with a high work function to facilitate hole injection, such as an alloy or a conductive compound. The first electrode 120 may be a reflective electrode, a semi-transmissive electrode, or a transmissive electrode. The first electrode 120 may have a single-layered structure, or a multi-layered structure including two or more layers. For example, the first electrode 120 may be a transparent electrode formed of indium tin oxide (ITO), indium zinc oxide (IZO), tin oxide (SnO₂), or zinc oxide (ZnO), which has excellent transparency and conductivity. On the transparent first electrode 120, magnesium (Mg), aluminum (Al), aluminum-lithium (Al—Li), calcium (Ca), magnesium-indium (Mg—In), or magnesium-silver (Mg—Ag) may be disposed, so as to form a reflective electrode. In an embodiment, the first electrode 120 may have a three-layered structure of ITO/Ag/ITO, but embodiments of the present disclosure are not limited thereto.

The hole transport region may be disposed on the first electrode 120.

The hole transport region may include at least one selected from selected from a hole injection layer 130, a hole transport layer 140, an electron blocking layer (not shown), and a buffer layer (not shown).

The hole transport region may include only either of a hole injection layer 130 or a hole transport layer 140. In an embodiment, the hole transport region may have a hole injection layer/hole transport layer structure or a hole injection layer/hole transport layer/electron blocking layer structure, wherein for each structure, constituting layers are sequentially stacked from the first electrode 120 in the stated order.

The hole injection layer 130 may include at least one selected from selected from, for example, poly(ether ketone)-containing triphenylamine (TPAPEK), 4-iso-propyl-4′-methyldiphenyliodonium tetrakis(pentafluorophenyl) borate (PPBI), N, N′-diphenyl-N, N′-bis-[4-(phenyl-m-tolyl-amino)-phenyl]-biphenyl-4,4′-diamine (DNTPD), copper phthalocyanine, 4,4′,4″-tris(3-methylphenylphenylamino) triphenylamine (m-MTDATA), N, N′-di(1-naphthyl)—N, N′-diphenylbenzidine (NPB), 4,4′,4″-tris(diphenylamino) triphenylamine (TDATA), 4,4′,4″-tris(N,N-2-naphthylphenylamino) triphenylamine (2-TNATA), polyaniline/dodecylbenzene sulphonic acid (PANI/DBSA), poly(3,4-ethylenedioxythiophene)/poly(4-styrene sulfonate) (PEDOT/PSS), polyaniline/10-camphor sulfonic acid (PANI/CSA), and polyaniline/poly(4-styrene sulfonate) (PANI/PSS).

The hole injection layer 130 may have a thickness in a range of about 10 nm to about 1,000 nm, for example, about 10 nm to about 100 nm.

The hole transport layer 140 may include at least one selected from selected from, for example, a carbazole derivative, such as 1,1-bis[(di-4-tolylamino)phenyl] cyclohexane (TAPC), N-phenylcarbazole, and polyvinylcarbazole, N,N′-bis(3-methylphenyl)-N,N′-diphenyl-[1,1-biphenyl]-4,4′-diamine (TPD), 4,4′,4″-tris(N-carbazolyl) triphenylamine (TCTA), N,N′-di(1-naphthyl)—N,N′-diphenylbenzidine (NPB), poly(9,9-dioctyl-fluorene-co-N-(4-butylphenyl)-diphenylamine (TFB), and amine-based polymer.

The hole transport layer 140 may have a thickness in a range of about 10 nm to about 1,000 nm, for example, about 10 nm to about 150 nm.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be one selected from a quinone derivative, a metal oxide, and a cyano group-containing compound, but embodiments of the present disclosure are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenium oxide; and a cyano group-containing compound, such as Compound HT-D1 or Compound HT-D2 below, but are not limited thereto.

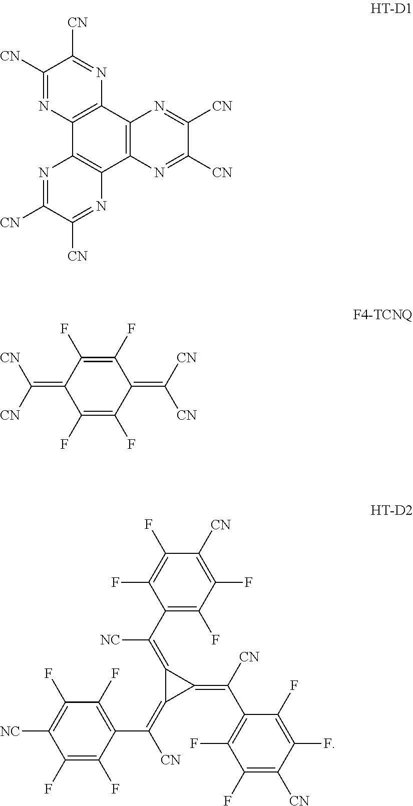

Meanwhile, when the hole transport region includes a buffer layer, a material for the buffer layer may be selected from materials for the hole transport region described above and materials for a host to be explained later, but embodiments of the present disclosure are not limited thereto.

In addition, when the hole transport region includes an electron blocking layer, a material for the electron blocking layer may be selected from materials for the hole transport region described above and materials for a host to be explained later, but embodiments of the present disclosure are not limited thereto. For example, when the hole transport region includes an electron blocking layer, a material for the electron blocking layer may be mCP.

The emission layer 150 may be formed on the hole transport region. The emission layer 150 is a layer that emits light by fluorescence or phosphorescence. The emission layer 150 may include a host and/or a dopant, and when included, the host may include the heterocyclic compound represented by Formula 1. In addition, the host and/or the dopant included in the emission layer 150 may be known materials.

For example, the host may include tris(8-quinolinato)aluminium (Alq₃), 4,4′-bis(carbazol-9-yl)biphenyl (CBP), poly(n-vinylcarbazole (PVK), 9,10-di(naphthalene)anthracene (ADN), 4,4′,4″-tris(N-carbazolyl)triphenylamine (TCTA), 1,3,5-tris(N-phenyl-benzimidazol-2-yl)benzene (TPBi), 3-tert-butyl-9,10-di(naphth-2-yl)anthracene (TBADN), distyrylarylene (DSA), 4,4′-bis(9-carbazole)-2,2′-dimethyl-bipheny (dmCBP), and the following compounds, but embodiments of the present disclosure are not limited thereto:

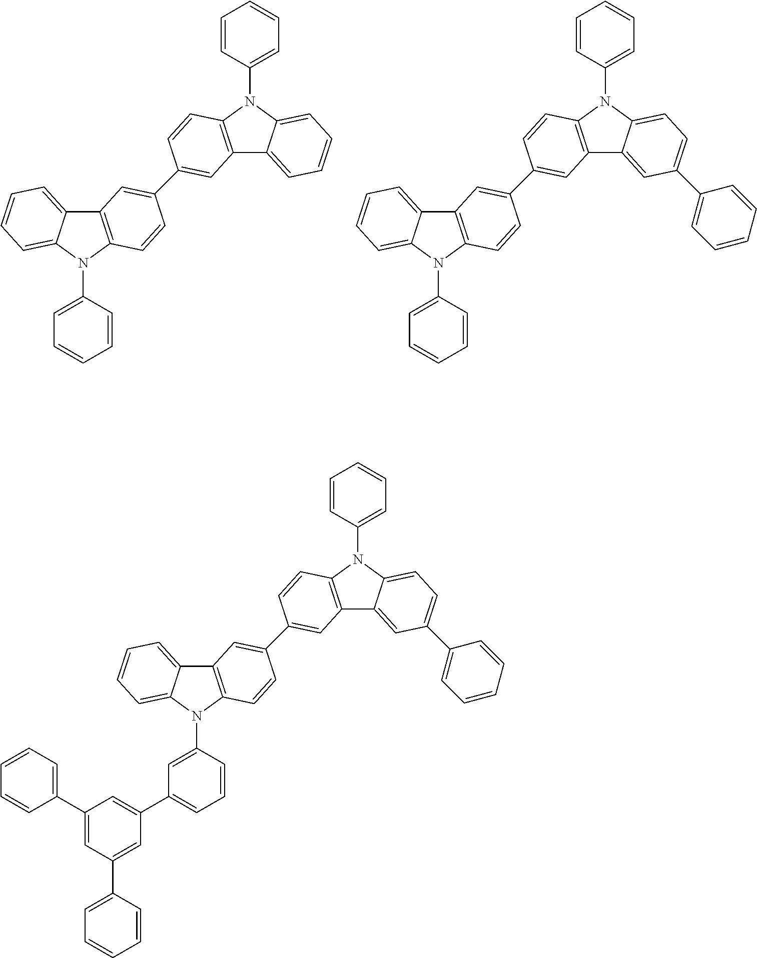 [SMILES: c1ccc(-n2c3ccccc3c3cc(-c4ccc5c(c4)c4ccccc4n5-c4ccccc4)ccc32)cc1 ; c1ccc(-c2ccc3c(c2)c2cc(-c4ccc5c(c4)c4ccccc4n5-c4ccccc4)ccc2n3-c2ccccc2)cc1 ; c1ccc(-c2cc(-c3ccccc3)cc(-c3cccc(-n4c5ccccc5c5cc(-c6ccc7c(c6)c6cc(-c8ccccc8)ccc6n7-c6ccccc6)ccc54)c3)c2)cc1]

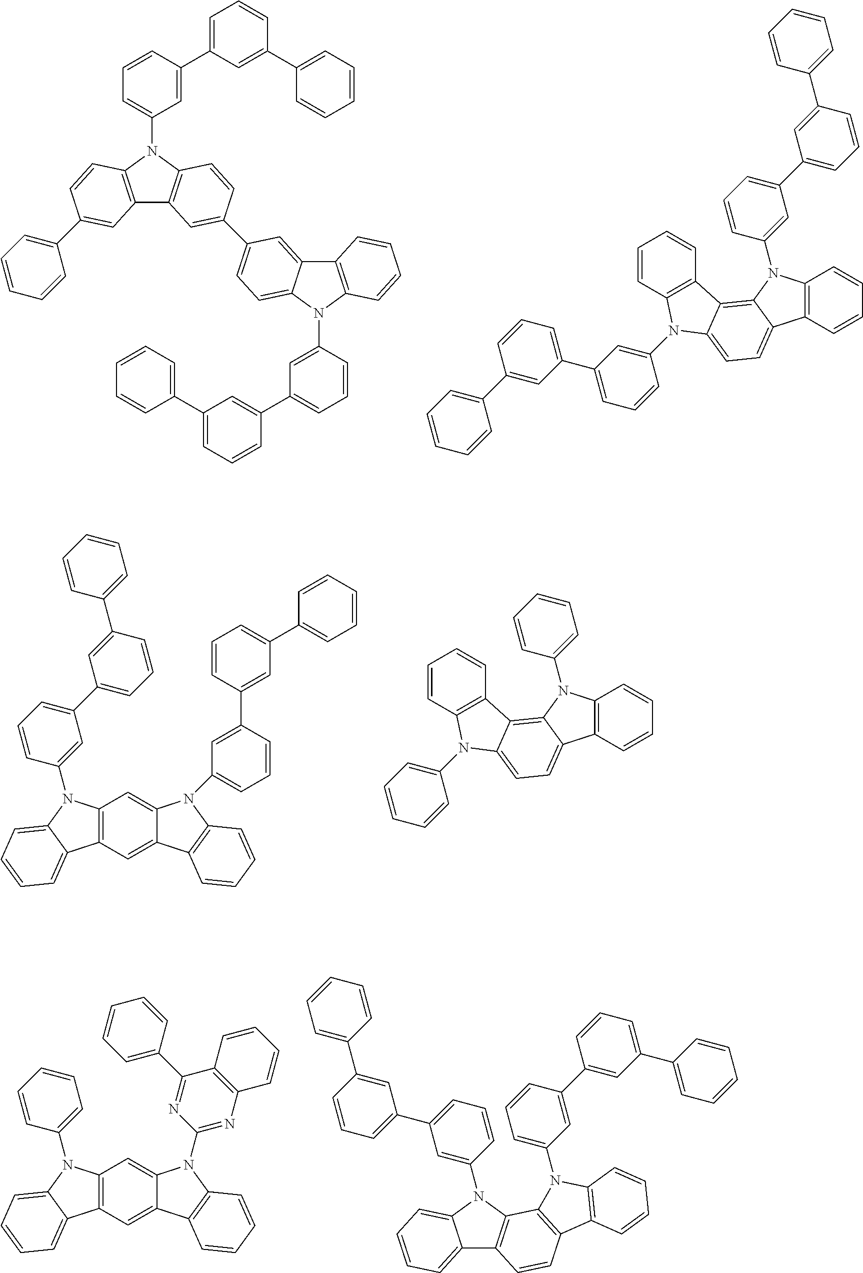

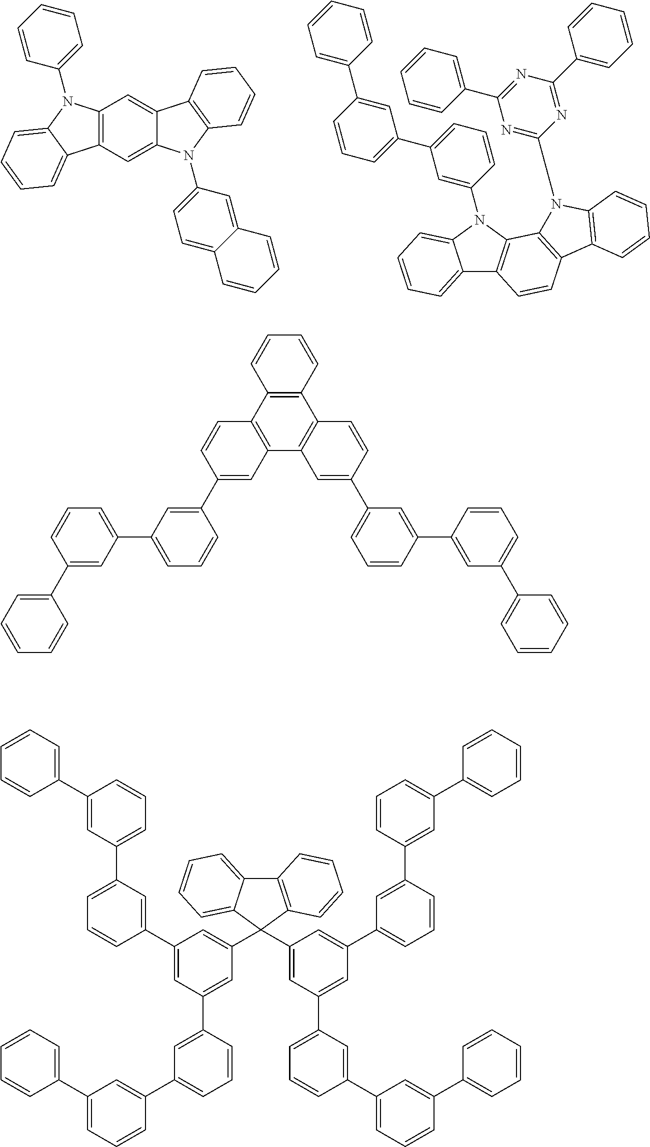

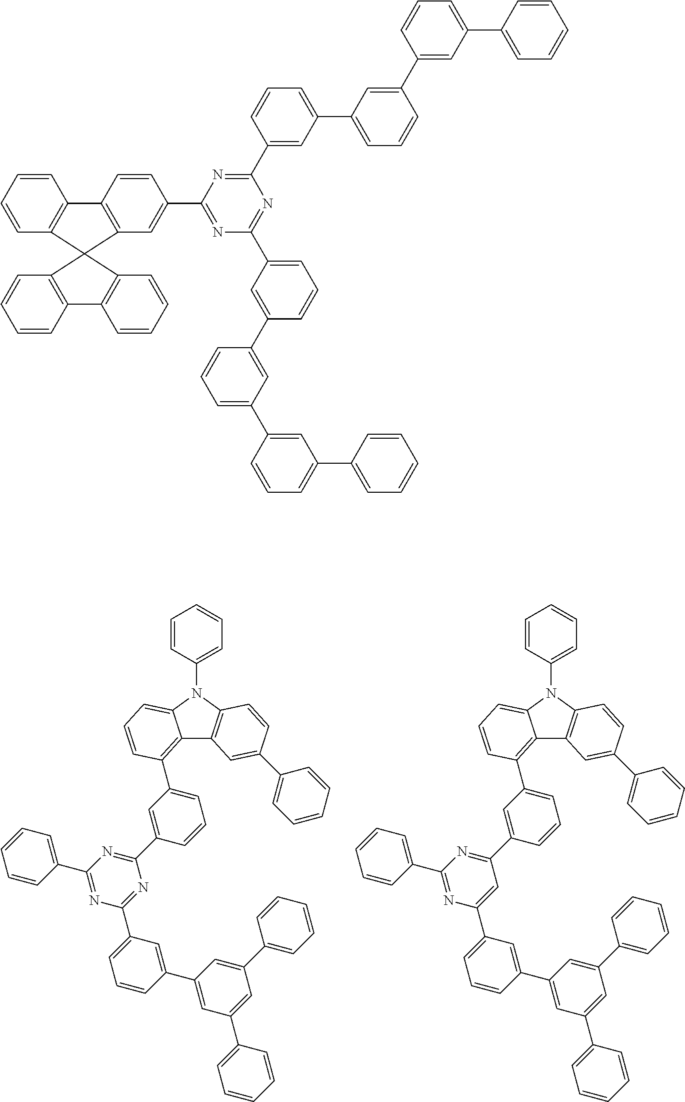 [SMILES: c1ccc(-c2cccc(-c3cccc(-c4cccc(-c5nc(-c6cccc(-c7cccc(-c8cccc(-c9ccccc9)c8)c7)c6)nc(-c6ccc7c(c6)C6(c8ccccc8-c8ccccc86)c6ccccc6-7)n5)c4)c3)c2)cc1 ; c1ccc(-c2cc(-c3ccccc3)cc(-c3cccc(-c4nc(-c5ccccc5)nc(-c5cccc(-c6cccc7c6c6cc(-c8ccccc8)ccc6n7-c6ccccc6)c5)n4)c3)c2)cc1 ; c1ccc(-c2cc(-c3ccccc3)cc(-c3cccc(-c4cc(-c5cccc(-c6cccc7c6c6cc(-c8ccccc8)ccc6n7-c6ccccc6)c5)nc(-c5ccccc5)n4)c3)c2)cc1]

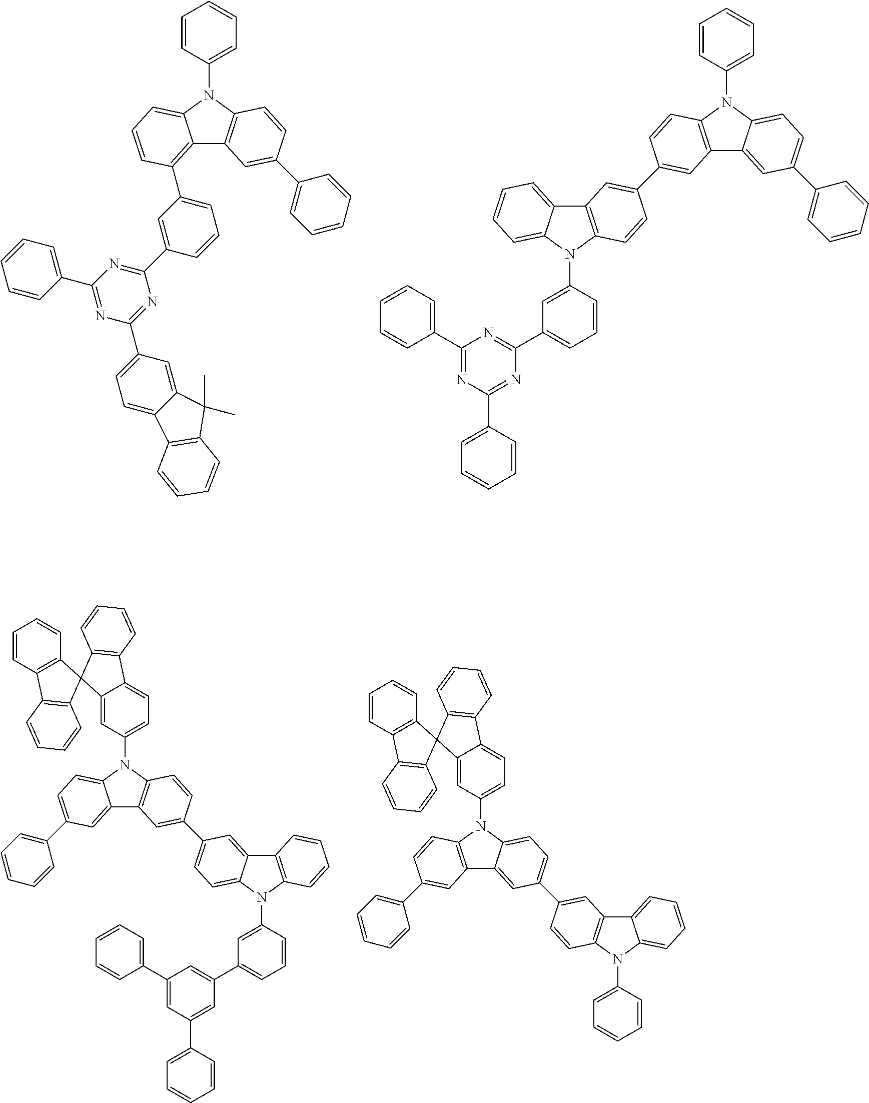 [SMILES: CC1(C)c2ccccc2-c2ccc(-c3nc(-c4ccccc4)nc(-c4cccc(-c5cccc6c5c5cc(-c7ccccc7)ccc5n6-c5ccccc5)c4)n3)cc21 ; c1ccc(-c2ccc3c(c2)c2cc(-c4ccc5c(c4)c4ccccc4n5-c4cccc(-c5nc(-c6ccccc6)nc(-c6ccccc6)n5)c4)ccc2n3-c2ccccc2)cc1 ; c1ccc(-c2cc(-c3ccccc3)cc(-c3cccc(-n4c5ccccc5c5cc(-c6ccc7c(c6)c6cc(-c8ccccc8)ccc6n7-c6ccc7c(c6)C6(c8ccccc8-c8ccccc86)c6ccccc6-7)ccc54)c3)c2)cc1 ; c1ccc(-c2ccc3c(c2)c2cc(-c4ccc5c(c4)c4ccccc4n5-c4ccccc4)ccc2n3-c2ccc3c(c2)C2(c4ccccc4-c4ccccc42)c2ccccc2-3)cc1]

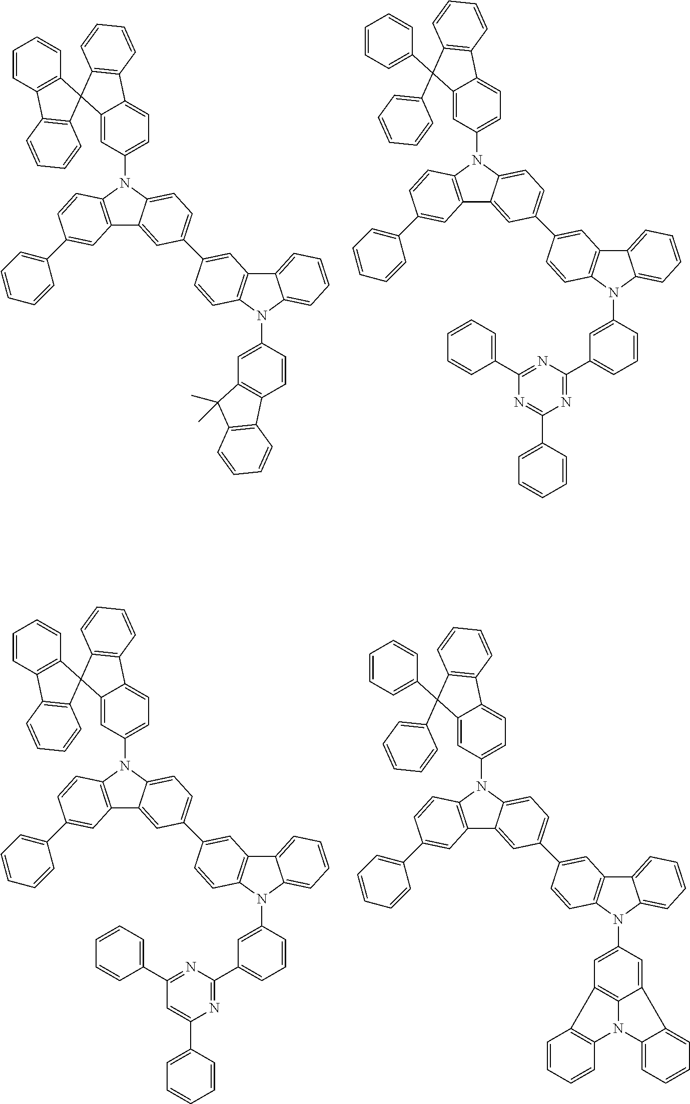

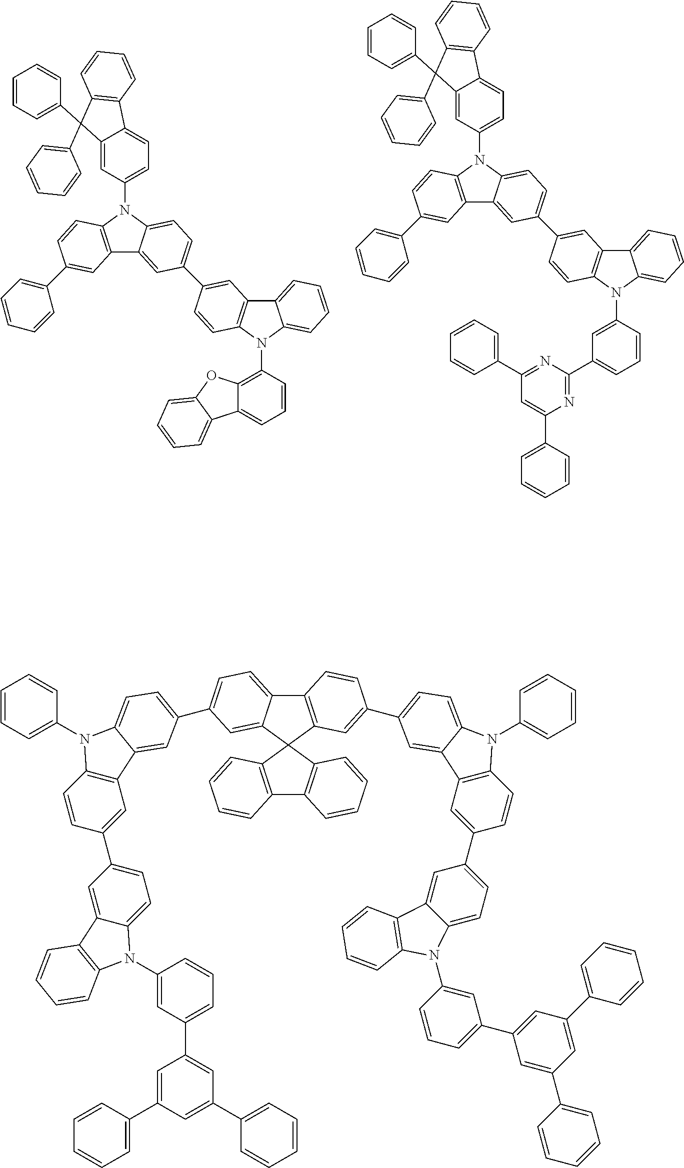

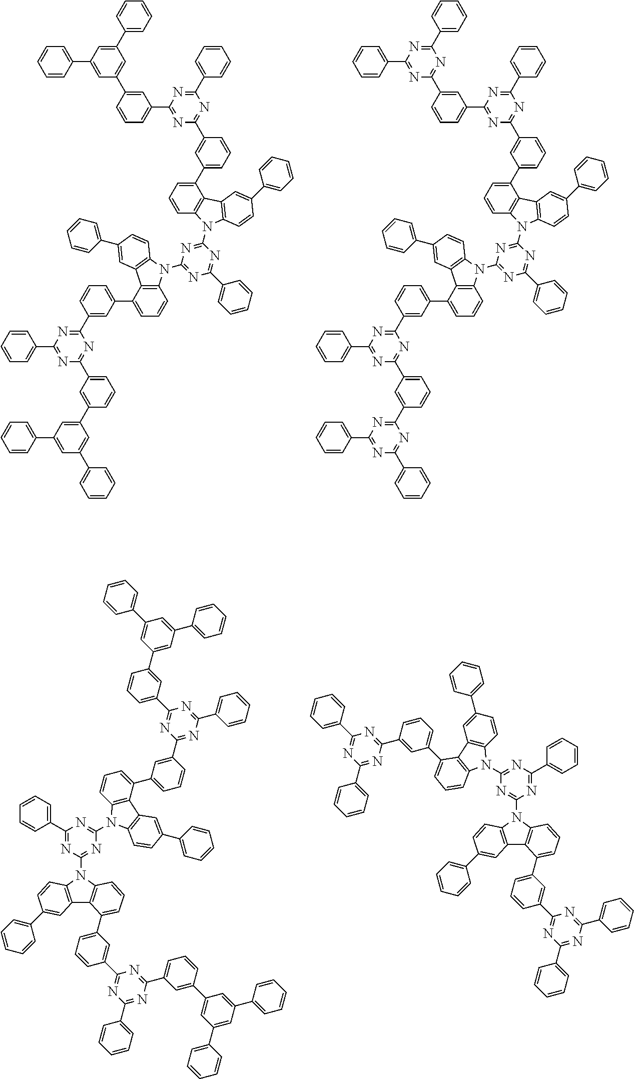

In an embodiment, the emission layer may include the heterocyclic compound represented by Formula 1, and may further include at least one selected from selected from a compound represented by Formula 4-1, a compound represented by Formula 4-2, a compound represented by Formula 5, a compound represented by Formula 6, and a compound represented by Formula 7.

For example, the dopant may include a perylene and a derivative thereof, a rubrene and a derivative thereof, a coumarin and a derivative thereof, 4-dicyanomethylene-2-(p-dimethylaminostyryl)-6-methyl-4H-pyran (DCM) and a derivative thereof, an iridium complex, such as bis[2-(4,6-difluorophenyl)pyridinate] picolinate iridium (III) (Flrpic), bis(1-phenylisoquinoline)(acetylacetonate) iridium (III) (Ir(piq)₂(acac)), tris(2-phenylpyridine) iridium (III) (Ir(ppy)3) or tris(2-(3-p-xylyl)phenyl)pyridine iridium (III) (dopant), an osmium complex, or a platinum complex, but embodiments of the present disclosure are not limited thereto.

When the emission layer includes a host and a dopant, an amount of the dopant may be 0.01 parts to about 15 parts by weight based on 100 parts by weight of the host material, but embodiments of the present disclosure are not limited thereto.

The emission layer 150 may have a thickness in a range of about 10 nm to about 60 nm.

When the organic light-emitting device is a full-color organic light-emitting device, the emission layer may be patterned into a red emission layer, a green emission layer, and a blue emission layer. In one or more embodiments, due to a stacked structure including a red emission layer, a green emission layer, and/or a blue emission layer, the emission layer may emit white light.

The electron transport region may be formed on the emission layer 150.

The electron transport region may include at least one selected from a hole blocking layer (not shown), an electron transport layer 160, and an electron injection layer 170.

For example, the electron transport region may have a hole blocking layer/electron transport layer/electron injection layer structure or an electron transport layer/electron injection layer structure, but the structure of the electron transport region is not limited thereto. The electron transport layer may have a single-layered structure or a multi-layered structure including two or more different materials.

For example, the organic light-emitting device 100 may include, to prevent the excitons or holes from diffusing into the electron transport layer 160, a hole blocking layer between the electron transport layer 160 and the emission layer 150. The hole blocking layer may include, for example, at least one selected from an oxadiazole derivative, a triazole derivative, a phenanthroline derivative, BCP, Bphen, and BAlq, but embodiments of the present disclosure are not limited thereto.

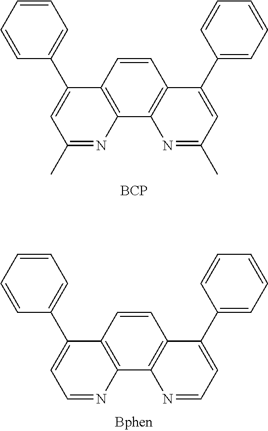

A thickness of the hole blocking layer may be in a range of about 20 Angstroms (Å) to about 1,000 Å, for example, about 30 Å to about 300 Å. While not wishing to be bound to theory, it is understood that when the thickness of the hole blocking layer is within these ranges, the hole blocking layer may have excellent hole blocking characteristics without a substantial increase in driving voltage.

The electron transport layer 160 may include tris(8-quinolinato) aluminium (Alq₃), BAlq, a compound including a pyridine ring, such as 1,3,5-tri[(3-pyridyl)-phen-3-yl]benzene, a compound including a triazine ring, such as 2,4,6-tris(3′-(pyridin-3-yl)biphenyl-3-yl)-1,3,5-triazine, a compound including an imidazole ring, such as 2-(4-(N-phenylbenzimidazolyl-1-yl-phenyl)-9,10-dinaphthylanthracene, a compound including a triazole ring, such as TAZ and NTAZ, 1,3,5-tris(N-phenyl-benzimidazol-2-yl)benzene (TPBi), BCP, or Bphen:

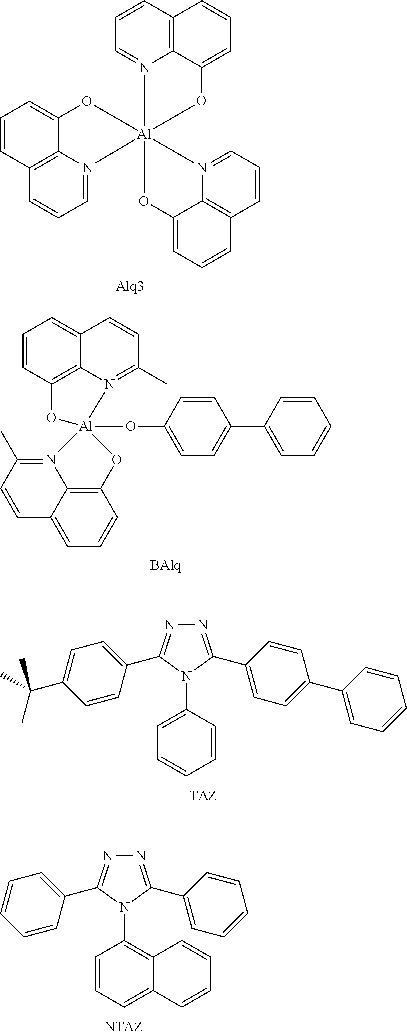

In one or more embodiments, the electron transport layer 160 may include a commercial product, such as KLET-01, KLET-02, KLET-03, KLET-10, or KLET-M1 (these products are available from Chemipro Kasei).

The electron transport layer 160 may further include, in addition to the materials described above, a metal-containing material.

The metal-containing material may include a Li complex. The Li complex may include, for example, Compound ET-D1 (lithium 8-hydroxyquinolate, LiQ) or ET-D2:

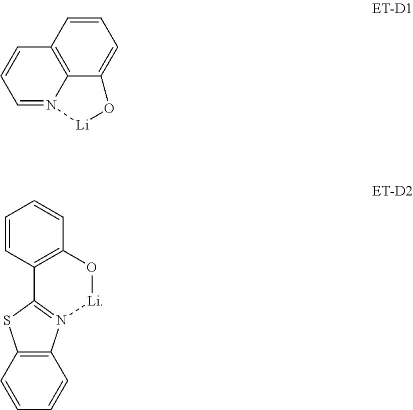

The electron transport layer 160 may be formed to a thickness, for example, in a range of about 15 nm to about 50 nm.

The electron injection layer 170 may be formed on the electron transport layer 160.

The electron injection layer 170 may include, for example, an lithium compound, such as (8-hydroxyquinolinato)lithium (LiQ) and lithium fluoride (LiF), sodium chloride (NaCl), cesium fluoride (CsF), lithium oxide (Li₂O), or barium oxide (BaO).

The electron injection layer 170 may be formed to a thickness in a range of about 0.3 nm to about 9 nm.

The second electrode 180 may be formed on the electron injection layer 170. The second electrode 180 may be a cathode and may be formed by using a material having a low work function among a metal, an alloy, an electrically conductive compound, and any combination thereof. For example, the second electrode 180 may be formed as a reflective electrode by using a metal such as lithium (Li), magnesium (Mg), aluminum (Al), and calcium (Ca), or an alloy such as aluminum-lithium (Al—Li), magnesium-indium (Mg—In), and magnesium-silver (Mg—Ag). Alternatively, the second electrode 180 may be formed as a transparent electrode by using the metal or the alloy thin film having a thickness of 20 nm or less, or a transparent conductive film such as indium tin oxide (In₂O₃—SnO₂) and indium zinc oxide (In₂O₃—ZnO).

In addition, the stacked structure of the organic light-emitting device 100 according to the embodiment is not limited to the above-described stacked structure. The organic light-emitting device 100 according to the embodiment may be formed in other known stacked structures. For example, in the organic light-emitting device 100, at least one of the hole injection layer 130, the hole transport layer 140, the electron transport layer 160, and the electron injection layer 170 may be omitted. The organic light-emitting device 100 may further include another layer. In addition, each layer of the organic light-emitting device 100 may be a single layer or a multi-layer.

A method of manufacturing each layer of the organic light-emitting device 100 according to the embodiment is not particularly limited. For example, each layer of the organic light-emitting device 100 according to the embodiment may be manufactured by using various methods, such as vacuum deposition, solution coating, and Langmuir-Blodgett (LB) deposition.

The solution coating may include spin coating, casting, micro gravure coating, gravure coating, bar coating, roll coating, wire bar coating, dip coating, spry coating, screen printing, flexographic printing, offset printing, and ink-jet printing.

Examples of the solvent used in the solution coating may include toluene, xylene, diethyl ether, chloroform, ethyl acetate, dichloromethane, tetrahydrofuran, acetone, acetonitrile, N, N-dimethylformamide, dimethylsulfoxide, anisole, hexamethylphosphoric acid triamide, 1,2-dichloroethane, 1,1,2-trichloroethane, chlorobenzene, o-dichlorobenzene, dioxane, cyclohexane, n-pentane, n-hexane, n-heptane, n-octane, n-nonane, n-decane, methyl ethyl ketone, cyclohexanone, butyl acetate, ethyl cellosolve acetate, ethylene glycol, ethylene glycol monobutyl ether, ethylene glycol monoethyl ether, ethylene glycol monomethyl ether, dimethoxy ethane, propylene glycol, diethoxymethane, triethylene glycol monoethyl ether, glycerin, 1,2-hexanediol, methanol, ethanol, propanol, iso-propanol, cyclohexanal, and N-methyl-2-pyrrolidone, but the solvent is not limited as long as the solvent can dissolve the material used to form each layer.

Considering the coatability, the concentration of the composition used in the solution coating may be in a range from 0.1 weight % to 10 weight %, for example, in a range from 0.5 weight % to 5 weight %, but embodiments of the present disclosure are not limited thereto.

The compound used in the vacuum deposition may be different according to the structure and thermal characteristics of the target layer, but may be selected from, for example, a deposition temperature of about 100° C. to about 500° C., a vacuum degree of about 10⁻⁸ torr to about 10⁻³ torr, a deposition rate of about 0.01 Angstroms per second (Å/sec) to about 100 Å/sec.

In an embodiment, the first electrode 120 may be an anode, and the second electrode 180 may be a cathode.

For example, the first electrode 120 may be an anode; the second electrode 180 may be a cathode; the organic layer may include the emission layer 150 between the first electrode 120 and the second electrode 180; the organic layer may further include a hole transport region between the first electrode 120 and the emission layer 150 and an electron transport region between the emission layer 150 and the second electrode 180; the hole transport region may include at least one selected from a hole injection layer 130, a hole transport layer 140, a buffer layer, and an electron blocking layer; and the electron transport region may include at least one selected from a hole blocking layer, an electron transport layer 160, and an electron injection layer 170.

In one or more embodiments, the first electrode 120 may be a cathode, and the second electrode 180 may be an anode.

Hereinbefore, the organic light-emitting device has been described with reference to the FIGURE, but embodiments of the present disclosure are not limited thereto.

Description of Substituents

The expression “X and Y may each independently be” as used herein refers to a case where X and Y may be identical to each other, or a case where X and Y may be different from each other.

The term “substituted” as used herein refers to a case where hydrogen of a substituent such as Ri may be further substituted with other substituents.

The term “C₁-C₆₀ alkyl group” as used herein refers to a linear or branched aliphatic saturated hydrocarbon monovalent group having 1 to 60 carbon atoms, and examples thereof include a methyl group, an ethyl group, an n-propyl group, an iso-propyl group, an n-butyl group, an iso-butyl group, a sec-butyl group, a tert-butyl group, an n-pentyl group, an iso-pentyl group, a tert-pentyl group, a neo-pentyl group, a 1,2-dimethylpropyl group, an n-hexyl group, an iso-hexyl group, a 1,3-dimethylbutyl group, a 1-iso-propylpropyl group, a 1,2-dimethylbutyl group, an n-heptyl group, a 1,4-dimethylpentyl group, a 3-ethylpentyl group, a 2-methyl-1-iso-propylpropyl group, a 1-ethyl-3-methylbutyl group, an n-octyl group, a 2-ethylhexyl group, a 3-methyl-1-iso-propylbutyl group, a 2-methyl-1- iso-propyl group, a 1-tert-butyl-2-methylpropyl group, an n-nonyl group, a 3,5,5-trimethyldecyl group, an n-decyl group, an isodecyl group, an n-undecyl group, a 1-methyldecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, an n-eicosyl group, an n-heneicosyl group, an n-docosyl group, an n-tricosyl group, and an n-tetracosyl group.

The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by —OA₁₀₁ (wherein Aioi is the C₁-C₆₀ alkyl group), and examples thereof include a methoxy group, an ethoxy group, a propoxy group, an iso-propoxy group, an n-butoxy group, an iso-butoxy group, a sec-butoxy group, a tert-butoxy group, an n-pentoxy group, an iso-pentoxy group, a tert-pentoxy group, a neo-pentoxy group, an n-hexyloxy group, an iso-hexyloxy group, a heptyloxy group, an octyloxy group, a nonyloxy group, a decyloxy group, an undeoxy group, a dodecyloxy group, a tridecyloxy group, a tetradecyloxy group, a pentadecyloxy group, a hexadecyloxy group, a heptadecyloxy group, an octadecyloxy group, a 2-ethylhexyloxy group, and a 3-ethylpentyloxy group.

The term “C₁-C₆₀ alkylthio group” as used herein refers to a monovalent group represented by —SA₁₀₂ (wherein A₁₀₂ is the C₁-C₆₀ alkyl group).

The term “C₃-C₁₀ cycloalkyl group” as used herein refers to a monovalent saturated hydrocarbon monocyclic group having 3 to 10 carbon atoms involved in the ring formation, and examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₆-C₆₀ aryl group” as used herein refers to a monovalent group having a carbocyclic aromatic system having 6 to 60 carbon atoms involved in the ring formation (that is, when substituted with a substituent, the atom not included in the substituent is not counted as the carbon involved in the ring formation), and the term “C₆-C₆₀ arylene group” as used herein refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₆-C₆₀ aryloxy group” as used herein refers to a group represented by —OA₁₀₃ (wherein A₁₀₃ is the C₆-C₆₀ aryl group), and examples thereof include a 1-naphthyloxy group, a 2-naphthyloxy group, and a 2-azulenyloxy group.

The term “C₆-C₆₀ arylthio group” as used herein refers to a group represented by —SA₁₀₄ (wherein A₁₀₄ is the C₆-C₆₀ aryl group).

The term “C₁-C₆₀ heteroaryl group” as used herein refers to a monovalent group having a heterocyclic aromatic system that has at least one heteroatom selected from N, O, Si, P, and S as a ring-forming atom, and 1 to 60 carbon atoms. The term “C₁-C₆₀ heteroarylene group” as used herein refers to a divalent group having a heterocyclic aromatic system that has at least one heteroatom selected from N, O, Si, P, and S as a ring-forming atom, and 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, the rings may be fused to each other.

The term “C₁-C₆₀ heteroaryloxy group” as used herein refers to a monovalent group represented by —OA₁₀₅ (wherein A₁₀₅ is the C₁-C₆₀ heteroaryl group), and examples thereof include a 2-furanyloxy group, a 2-thienyloxy group, a 2-indolyloxy group, a 3-indolyloxy group, a 2-benzofuriloxy group, and a 2-benzothienyloxy group.

The term “C₁-C₆₀ heteroarylthio group” as used herein refers to a group represented by —SA₁₀₆ (wherein A₁₀₆ is the C₁-C₆₀ heteroaryl group).

The term “C₇-C₃₀ arylalkyl group” as used herein refers to an aryl group substituted with an alkyl group, and is a monovalent group in which the sum of carbon atoms in the alkyl group and the aryl group that constitute the C₇-C₃₀ arylalkyl group is in a range of 7 to 30. Examples of the C₇-C₃₀ aryl alkyl group include a benzyl group, a phenylethyl group, a phenylpropyl group, and a naphthylmethyl group.

The term “C₆-C₃₀ arylalkyl” as used herein refers to a group represented by —OA₁₀₅ (wherein A₁₀₅ is the C₇-C₃₀ arylalkyl group).

The term “C₆-C₃₀ arylalkylthio group” as used herein refers to a group represented by —SA₁₀₆ (wherein A₁₀₆ is the C₇-C₃₀ arylalkyl group).

The term “C₈-C₃₀ arylalkenyl group” as used herein refers to an alkenyl group substituted with an aryl group, and is a monovalent group in which the sum of carbon atoms in the alkenyl group and the aryl group that constitute the C₈-C₃₀ arylalkenyl group is in a range of 8 to 30.

The term “monovalent non-aromatic condensed polycyclic group” as used herein refers to a monovalent group (for example, having 8 to 60 carbon atoms) having two or more rings condensed to each other, only carbon atoms as ring-forming atoms, and non-aromaticity in its entire molecular structure. Examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group (for example, having 1 to 60 carbon atoms) having two or more rings condensed to each other, has a heteroatom selected from N, O, P, Si, and S, other than carbon atoms, as a ring-forming atom, and no aromaticity in its entire molecular structure. Examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group.

The term “C₅-C₃₀ carbocyclic group” as used herein a saturated or unsaturated cyclic group having 5 to 30 carbon atoms only as ring-forming atoms. The C₅-C₃₀ carbocyclic group may be a monocyclic group or a polycyclic group, and according to its chemical structure, may be a monovalent, divalent, trivalent, tetravalent, pentavalent, or hexavalent group.

The term “C₁-C₃₀ heterocyclic group” as used herein a saturated or unsaturated cyclic group having, in addition to 1 to 30 carbon atoms as ring-forming atoms, at least one selected from hetero atom selected from N, O, P, Si, and S. The C₁-C₃₀ heterocyclic group may be a monocyclic group or a polycyclic group, and according to its chemical structure, may be a monovalent, divalent, trivalent, tetravalent, pentavalent, or hexavalent group.

At least one selected from substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —P(═O)(Q₁₈)(Q₁₉), and —C(═O)(Q₂₀);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —P(═O)(Q₂₈)(Q₂₉), and —C(═O)(Q₃₀); and —N(Q₃₁)(Q₃₂), —Si(Q₃₃)(Q₃₄)(Q₃₅), —B(Q₃₆)(Q₃₇), —P(═O)(Q₃₈)(Q₃₉) and —C(═O)(Q₄₀), and

Q₁ to Q_{10,} Q₁₁ to Q_{20,} Q₂₁ to Q_{30,} and Q₃₁ to Q₄₀ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one selected from a C₁-C₆₀ alkyl group, and a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

Others

The expression “A to B” as used herein refers to a range from A to B, including A and B.

While the embodiments of the present disclose have been described with reference to the accompanying drawings, it is understood that the present disclosure is not limited to these embodiments. It is apparent to those of ordinary skill in the art that various modifications or changes may be made thereto without departing from the spirit and scope of the appended claims. It is understood that various modifications or changes fall within the technical scope of the present disclosure.

Hereinafter, a condensed cyclic compound represented by Formula 1 and an organic light-emitting device including the same will be described in detail with reference to Examples and Comparative Examples. Examples provided below are merely an example, and the condensed cyclic compound and the organic light-emitting device, according to embodiments, are not limited to Examples provided below.

The expression “B was used instead of A” used in describing Synthesis Examples means that a molar equivalent of “A” was identical to a molar equivalent of

In addition, “%” is weight % unless specified otherwise.

### EXAMPLES

Hereinafter, a material for a light-emitting device and an organic light-emitting device according to embodiments are described in detail with reference to Examples and Comparative Examples. Examples are merely an example, and the material for the light-emitting device and the organic light-emitting device are not limited thereto.

### Synthesis Example 1

### Synthesis of Compound 1

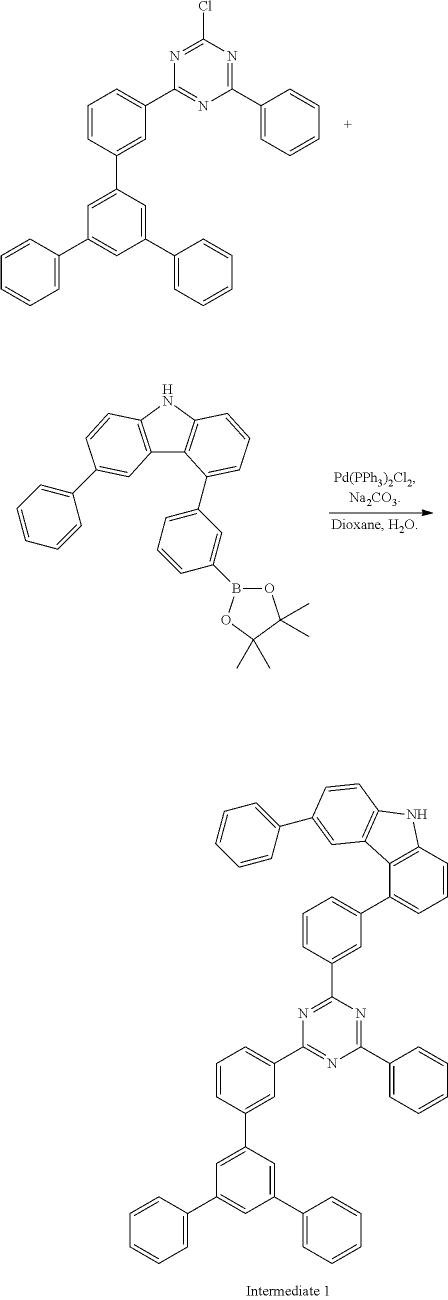 [SMILES: Clc1nc(-c2ccccc2)nc(-c2cccc(-c3cc(-c4ccccc4)cc(-c4ccccc4)c3)c2)n1 ; CC1(C)OB(c2cccc(-c3cccc4[nH]c5ccc(-c6ccccc6)cc5c34)c2)OC1(C)C ; c1ccc(-c2cc(-c3ccccc3)cc(-c3cccc(-c4nc(-c5ccccc5)nc(-c5cccc(-c6cccc7[nH]c8ccc(-c9ccccc9)cc8c67)c5)n4)c3)c2)cc1]

(1) Synthesis of Intermediate 1

In an argon atmosphere, 2-chloro-4-phenyl-6(5′-phenyl[1,1′:3′,1″-terphenyl]-3-yl)-1,3,5-triazine (4.00 grams (g), 7.67 millimoles, mmol), 3-phenyl-5-(4,4,5,5,-tetramethyl-1,3,2-dioxaborane-2-yl)phenyl)-9H-carbazole (2.95 g, 7.67 mmol), bi(triphenylphosphine)palladium(II) dichloride (0.11 g, 0.15 mmol), sodium carbonate (2.03 g, 19.18 mmol), dioxane (77 milliliters, ml), and water (38 ml) were added to a reaction container and mixed. Then, the mixture was stirred at a temperature of 85° C. for 4 hours. After the reaction was completed, the reaction mixture was cooled to room temperature, and the impurities were filtered by using Celite (registered trademark). Then, the solvent was distilled, purified by column chromatography, and dispersed and washed by using methanol to obtain Intermediate 1 (2.92 g).

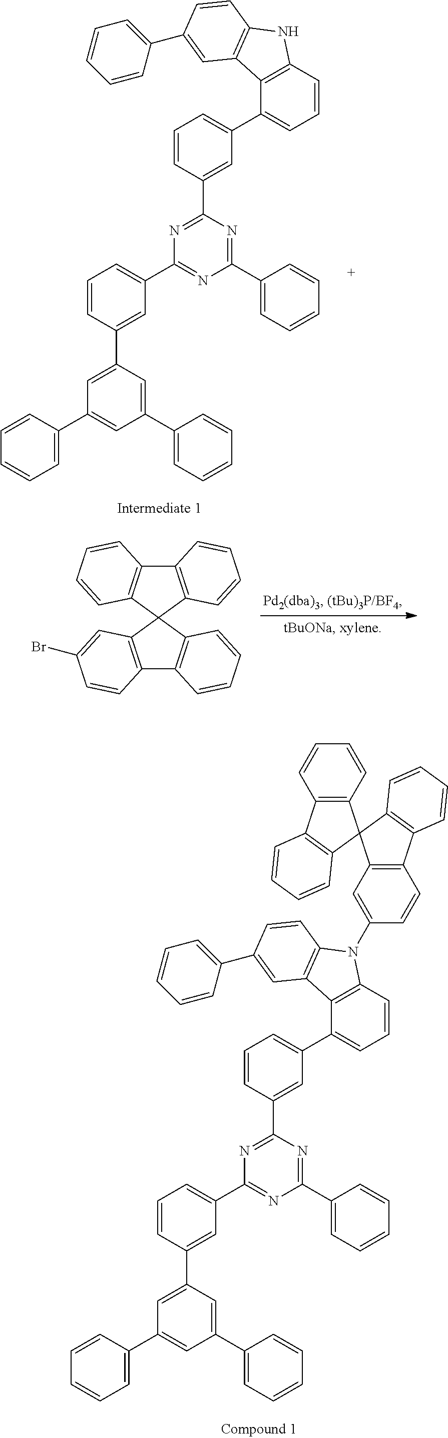 [SMILES: c1ccc(-c2cc(-c3ccccc3)cc(-c3cccc(-c4nc(-c5ccccc5)nc(-c5cccc(-c6cccc7[nH]c8ccc(-c9ccccc9)cc8c67)c5)n4)c3)c2)cc1 ; Brc1ccc2c(c1)C1(c3ccccc3-c3ccccc31)c1ccccc1-2 ; c1ccc(-c2cc(-c3ccccc3)cc(-c3cccc(-c4nc(-c5ccccc5)nc(-c5cccc(-c6cccc7c6c6cc(-c8ccccc8)ccc6n7-c6ccc7c(c6)C6(c8ccccc8-c8ccccc86)c6ccccc6-7)c5)n4)c3)c2)cc1]

(2) Synthesis of Compound 1

In an argon atmosphere, Intermediate 1 (2.30 g, 2.95 mmol), 2-bromo-9,9′-spirobi[fluorene] (1.28 g, 3.25 mmol), tris(dibenzylideneacetone)dipalladium(0) (0.135 g, 0.15 mmol), (t-Bu)₃P/BF₄ (0.09 g, 0.30 mmol), sodium tert-butoxide (0.85 g, 8.86 mmol), and xylene (46 ml) were added to a reaction container and mixed. Then, the mixture was stirred at a temperature of 120° C. for 6 hours. After the reaction was completed, the reaction mixture was cooled to room temperature, and the impurities were filtered by using Celite (registered trademark). Then, the solvent was distilled and purified by column chromatography to obtain Compound 1 (6 g).

### Synthesis Example 2

### Synthesis of Compound 2

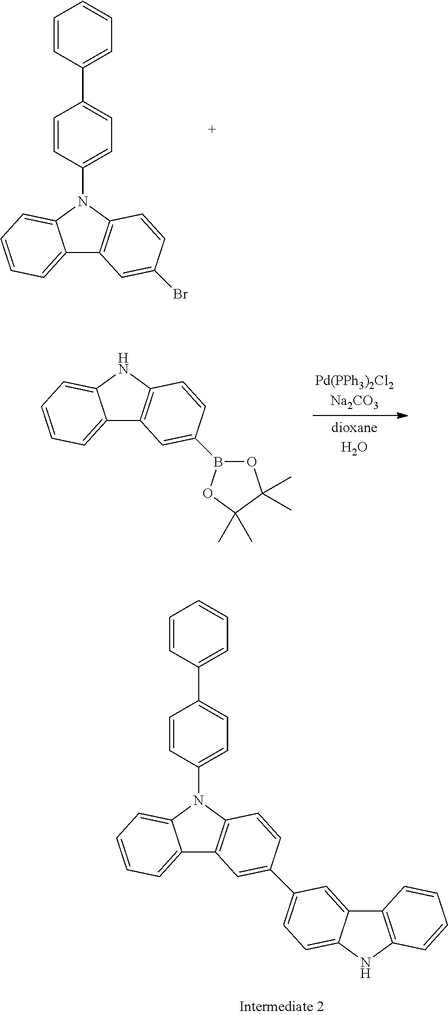 [SMILES: Brc1ccc2c(c1)c1ccccc1n2-c1ccc(-c2ccccc2)cc1 ; CC1(C)OB(c2ccc3[nH]c4ccccc4c3c2)OC1(C)C ; c1ccc(-c2ccc(-n3c4ccccc4c4cc(-c5ccc6[nH]c7ccccc7c6c5)ccc43)cc2)cc1]

(1) Synthesis of Intermediate 2

In an argon atmosphere, 9-(4-biphenyl-yl)-3-bromocarbazole (5.00 g, 12.55 mmol), 3-(4,4,5,5,-tetramethyl-1,3,2-dioxaborane-2-yl)carbazole (3.31 g, 11.30 mmol), bis(triphenylphosphine)palladium(II) dichloride (0.18 g, 0.25 mmol), sodium carbonate (3.33 g, 31. 38 mmol), dioxane (126 ml), and water (63 ml) were added to a reaction container and mixed. Then, the mixture was stirred at a temperature of 85° C. for 4 hours. After the reaction was completed, the reaction mixture was cooled to room temperature, and the impurities were filtered by using Celite (registered trademark). Then, the solvent was distilled and purified by column chromatography to obtain Intermediate 2 (6.5 g).

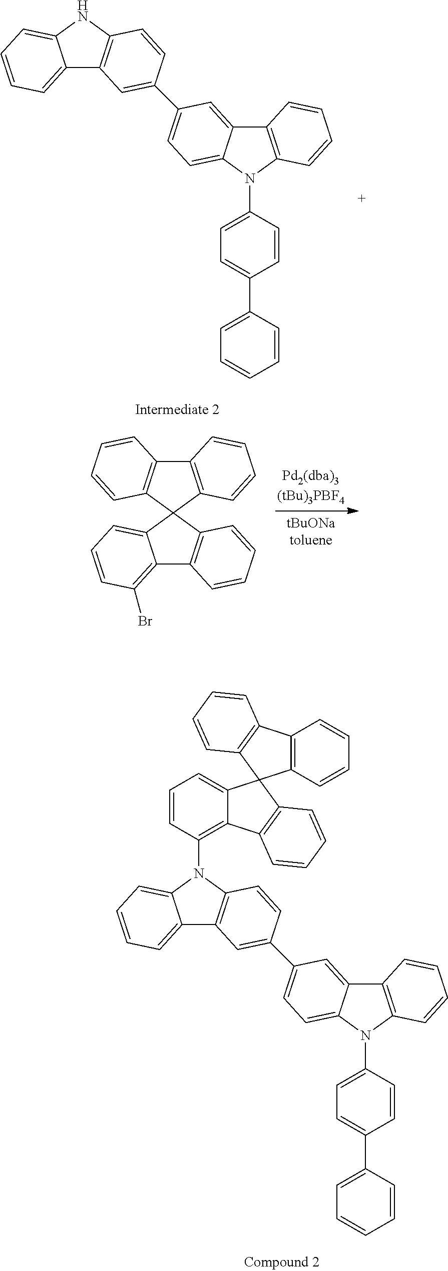 [SMILES: c1ccc(-c2ccc(-n3c4ccccc4c4cc(-c5ccc6[nH]c7ccccc7c6c5)ccc43)cc2)cc1 ; Brc1cccc2c1-c1ccccc1C21c2ccccc2-c2ccccc21 ; c1ccc(-c2ccc(-n3c4ccccc4c4cc(-c5ccc6c(c5)c5ccccc5n6-c5cccc6c5-c5ccccc5C65c6ccccc6-c6ccccc65)ccc43)cc2)cc1]

(2) Synthesis of Compound 2

In an argon atmosphere, Intermediate 2 (3.00 g, 6.19 mmol), 4-bromo-9,9′-spirobifluorene (2.20 g, 5.57 mmol), tris(dibenzylideneacetone)dipalladium(0) (0.113 g, 0.12 mmol), (t-Bu)₃P/BF₄ (0.14 g, 0.50 mmol), sodium tert-butoxide (0.89 g, 9.29 mmol), and xylene (26 ml) were added to a reaction container and mixed. Then, the mixture was stirred at a temperature of 120° C. for 6 hours. After the reaction was completed, the reaction mixture was cooled to room temperature, and the impurities were filtered by using Celite (registered trademark). Then, the solvent was distilled and purified by column chromatography to obtain Compound 2 (2 g).

### Examples 1 to 6 and Comparative Examples 1 to 3

As a first electrode (anode), an ITO glass substrate, on which a stripe-shaped ITO film having a thickness of 150 nanometers (nm) was deposited, was provided. PEDOT—PSS (manufactured by Sigma-Aldrich) was spin-coated on the ITO glass substrate to form a hole injection layer having a thickness of 15 nm.

Then, P-1 and FA-14 were dissolved in anisole to prepare a coating liquid for a hole transport layer. FA-14 was mixed with 20 percent by weight (weight %) based on a total weight of the hole transport layer. The coating liquid for the hole transport layer was spin-coated on the hole injection layer to form a hole transport layer having a thickness of 125 nm. Formulae of P-1 and FA-14 are as follows. P-1 was synthesized according to a synthesis method disclosed in International Publication No. WO 2011/159872, which is incorporated herein in its entirety by reference.

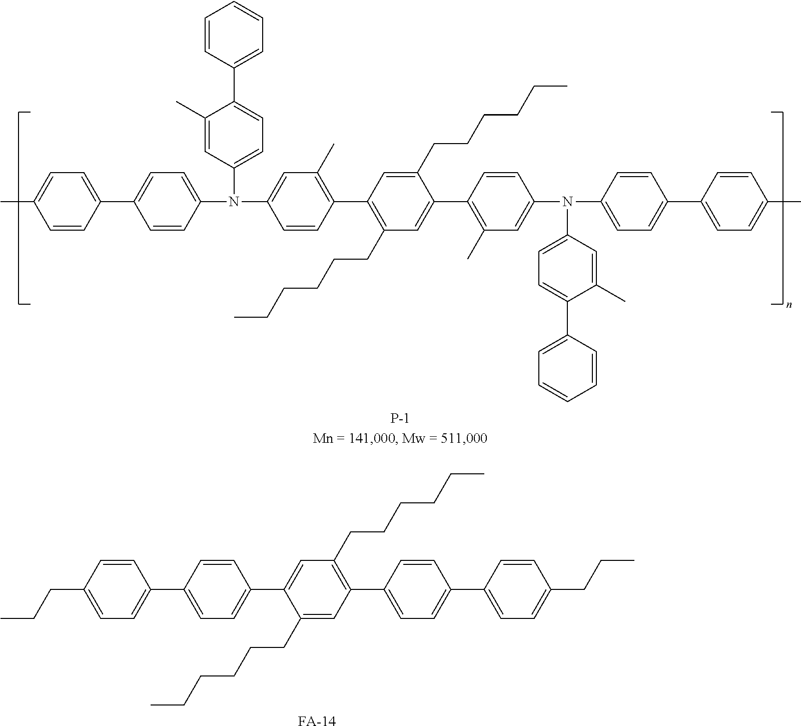

Then, a host material (see Table 1) and tris(2-(3-p-xylyl)phenyl)pyridine iridium (III) (TEG)) as a dopant material were dissolved in methyl benzoate to prepare a coating liquid for an emission layer. A doping amount of the dopant material was 10 weight % based on a total weight of the emission layer. Then, the coating liquid for the emission layer was spin-coated on the hole transport layer to form an emission layer having a thickness of 55 nm.

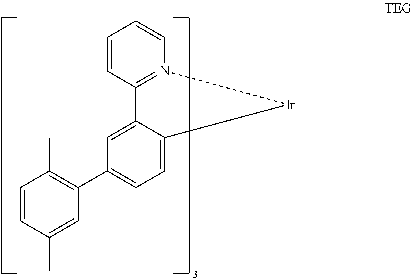

Lithium quinolate (LiQ) and KLET-03 (manufactured by Chemipro Kasei) were co-deposited on the emission layer at a weight ratio of 1:1 by vacuum deposition to form an electron transport layer having a thickness of 20 nm.

LiQ was vacuum-deposited on the electron transport layer to form an electron injection layer having a thickness of 3.5 nm.

Aluminum (Al) was vacuum-deposited on the electron injection layer to form a second electrode (cathode) having a thickness of 100 nm, thereby completing the manufacture of an organic light-emitting device.

Formulae of Compounds 3 to 7 used as the host material are as follows. Compounds 3 to 5 were synthesized according to synthesis methods disclosed in International Publication Nos. WO 2012/087955, WO 2016/033167, and WO 2016/033167, which are incorporated herein in their entireties by reference. In addition, Compounds 6 and 7 were synthesized according to a synthesis method disclosed in US Patent Application Publication No. 2017-174705, which is incorporated herein in its entirety by reference.

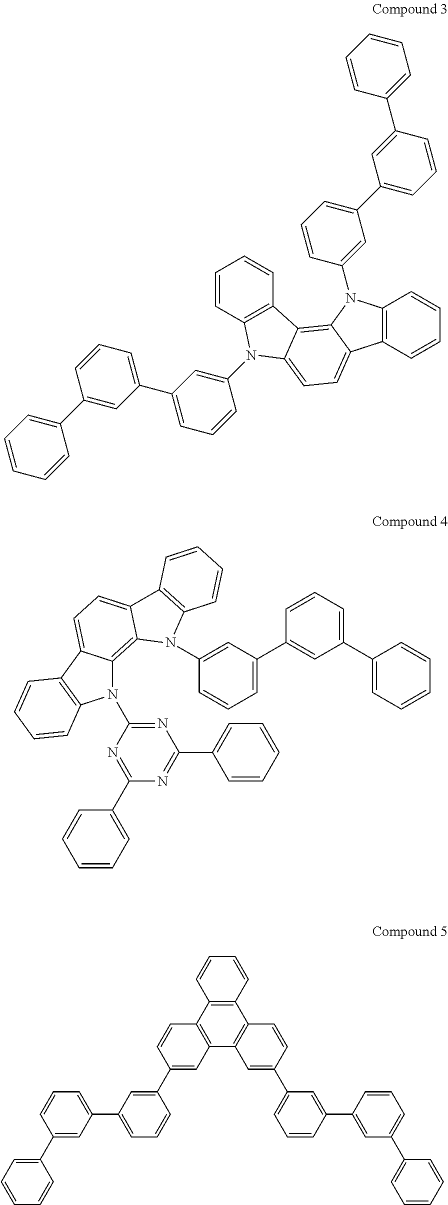

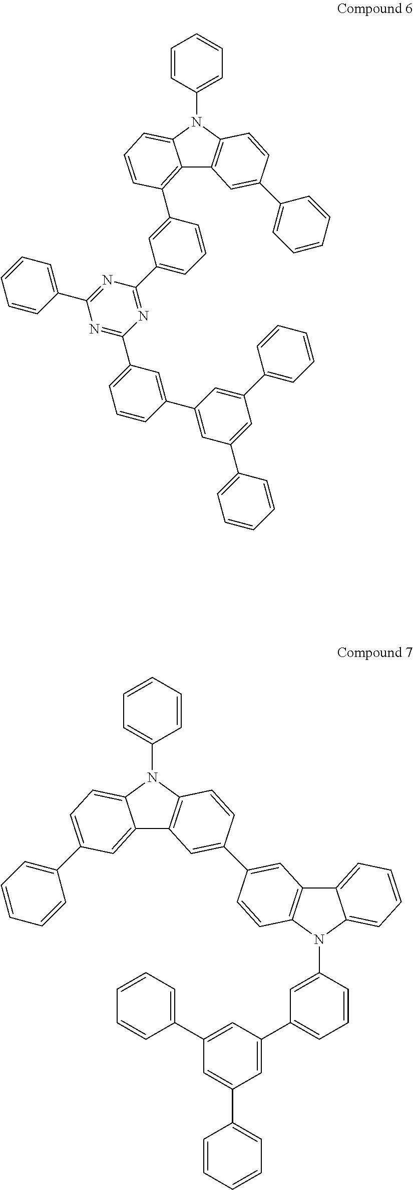 [SMILES: c1ccc(-c2cc(-c3ccccc3)cc(-c3cccc(-c4nc(-c5ccccc5)nc(-c5cccc(-c6cccc7c6c6cc(-c8ccccc8)ccc6n7-c6ccccc6)c5)n4)c3)c2)cc1 ; c1ccc(-c2cc(-c3ccccc3)cc(-c3cccc(-n4c5ccccc5c5cc(-c6ccc7c(c6)c6cc(-c8ccccc8)ccc6n7-c6ccccc6)ccc54)c3)c2)cc1]

Current Efficiency and Device Lifespan of Organic Light-Emitting Device

The current efficiency and device lifespan of the organic light-emitting devices of Examples 1 to 6 and Comparative Examples 1 to 3 were evaluated.

Each organic light-emitting device was caused to emit light by applying a predetermined voltage to each organic light-emitting device by using a DC constant voltage power source meter (manufactured by KEYENCE). At this time, the luminance of each organic light-emitting device was measured by using a luminance measurement device SR-3 (manufactured by Topcom). Then, the current density at which the luminance was 6,000 candelas per square meter (cd/m²) was measured while gradually increasing a current applied thereto. Then, the current was kept constant. The current efficiency was calculated at the obtained current density.

The emission lifespan indicates an amount of time (LT95) until the luminance value of the organic light-emitting device emitting light was 95% of initial luminance.

Table 1 shows evaluation results of the current efficiency and the emission lifespan of the organic light-emitting devices. In addition, the ratio of the host material in Table 1 refers to a weight ratio, and the current efficiency and the emission lifespan are relative values when the measured values of the organic light-emitting device of Comparative Example 1 are 100.

TABLE 1

| | | Current | |
|---|---|---|---|
| | | efficiency | Device lifespan |
| | Emission layer | (arbitrary unit) | (arbitrary unit) |
| Example 1 | Compound 1 | 123 | 150 |
| Example 2 | Compound 1:Compound | 121 | 400 |
| | 2 (5:5) | | |
| Example 3 | Compound 1:Compound | 126 | 250 |
| | 3 (5:5) | | |
| Example 4 | Compound 1:Compound | 120 | 300 |
| | 4 (5:5) | | |
| Example 5 | Compound 1:Compound | 125 | 300 |
| | 5 (7:3) | | |
| Example 6 | Compound 1:Compound | 121 | 500 |
| | 5 (3:7) | | |
| Example 7 | Compound 1:Compound | 122 | 700 |
| | 5 (2:8) | | |
| Comparative | Compound 6:Compound | 100 | 100 |
| Example 1 | 7 (5:5) | | |
| Comparative | Compound 6 | 28 | 10 |
| Example 2 | | | |

Referring to Table 1, it is confirmed that the organic light-emitting device of Example 1 using only Compound 1 has excellent current efficiency and device lifespan, as compared with the organic light-emitting device of Comparative Example 1 using Compound 6 and Compound 7 together and the organic light-emitting device of Comparative Example 2 using only Compound 6.

In addition, it is confirmed that the organic light-emitting devices of Examples 2 to 6 using Compound 1 and Compounds 2 to 5 together have more excellent device lifespan.

Glass Transition Temperature of Compounds and Solubility of Compounds to Methyl Benzoate

The glass transition temperature (T_{g}) of Compounds 1, 2, 6, and 7 and Compound A and the solubility of Compounds 1, 2, 6, and 7 and Compound A to methyl benzoate were measured.

The glass transition temperature (T_{g}) of Compounds was measured by using SII DSC₆₂₂₀ (manufactured by Seiko Instruments Inc.) based on WS K 7121.

In addition, the solubility of Compounds to methyl benzoate was measured by thoroughly mixing Compounds with methyl benzoate such that the concentration of Compound became a predetermined value at a temperature of 25° C. and visually observing the mixture. When the mixture was transparent, it was determined that Compound was dissolved, and when the mixture liquid was opaque, it is determined that Compound was not dissolved.

Table 2 shows evaluation results of T_{g} of Compounds 1, 2, 6, and 7 and Compound A and the solubility of Compounds 1, 2, 6, and 7 and Compound A to methyl benzoate.

TABLE 2

| | | Solubility to |
|---|---|---|
| | T_{g} | methyl benzoate |
| | (° C.) | (mass%) |
| Compound 1 | 178 | >4 |
| Compound 2 | 194 | >4 |
| Compound 6 | 146 | >4 |
| Compound 7 | 157 | >4 |
| Compound A | 120 | Not melted |

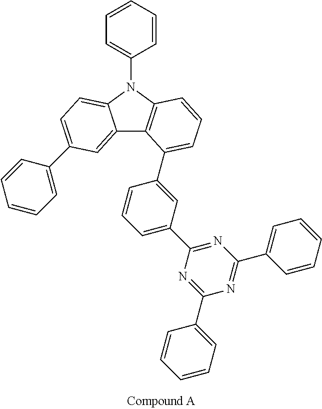 [SMILES: c1ccc(-c2ccc3c(c2)c2c(-c4cccc(-c5nc(-c6ccccc6)nc(-c6ccccc6)n5)c4)cccc2n3-c2ccccc2)cc1]

Referring to Table 2, it is confirmed that Compounds 1 and 2 have excellent heat resistance and solubility, as compared with Compounds 6 and 7 and Compound A, and that Compounds 1 and 2 are suitable for use as the material for the organic light-emitting device for solution coating.

Since the heterocyclic compound has improved electric characteristics and/or thermal stability, the organic light-emitting device including the heterocyclic compound has improved current efficiency and lifespan characteristics.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the present disclosure as defined by the following claims.

What is claimed is:

1. A heterocyclic compound represented by Formula 1:
(A)ₙ-L, Formula 1
wherein, in Formula 1, A is a group in which one hydrogen atom is excluded from a compound represented by Formula 1-1 or a group in which one hydrogen atom is excluded from a compound represented by Formula 1-2,
n is an integer from 1 to 8,
L is hydrogen, a single bond, or an n-valent linking group, and
when n is 1, L is hydrogen: 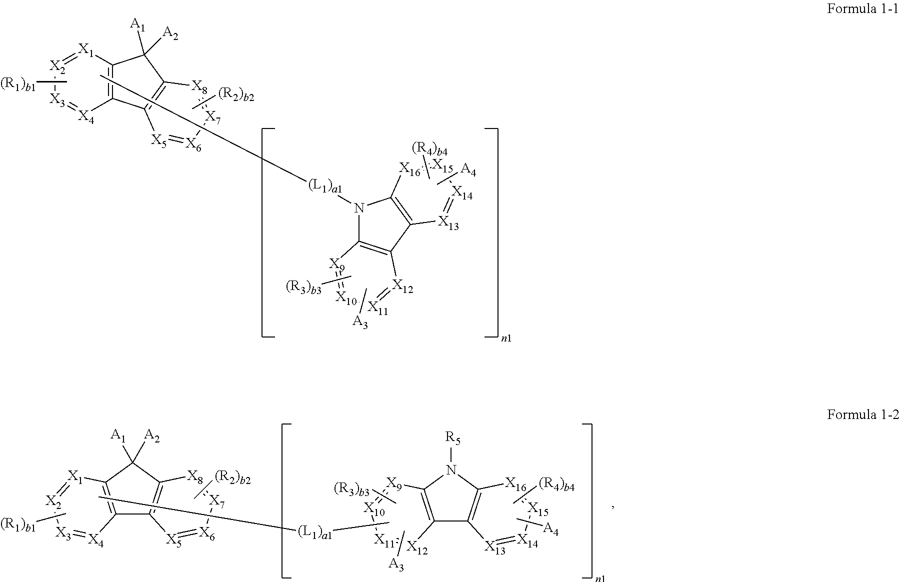
wherein, in Formulae 1-1 and 1-2,
X₁ to X₁₆ are each independently N or C,
at least one selected from X₁ to Xs is carbon linked to —(L₁)ₐ₁—,
in Formula 1-1, one selected from X₉ to X₁₂ is carbon linked to A₃, and one selected from X₁₃ to X₁₆ is carbon linked to A₄,
in Formula 1-2, one selected from X₉ to X₁₂ is carbon linked to —(L₁)ₐ₁—, and one selected from X₁₃ to X₁₆ is carbon linked to A₄,
A₁ and A₄ are each independently selected from hydrogen, a group represented by *—(L₂)ₐ₂-R_{ET}, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-Cso alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group,
at least one selected from A₃ and A₄ is a group represented by *—(L₂)ₐ₂—R_{ET},
A₁ and A₂ are optionally linked to form a saturated ring or an unsaturated ring,
L₁ and L₂ are each independently selected from a substituted or unsubstituted C₅-C₃₀ carbocyclic group and a substituted or unsubstituted C₁-C₃₀ heterocyclic group,
a1 and a2 are each independently an integer from 0 to 3, wherein, when a1 is 0, *—(L₁)ₐ₁—*′ is a single bond, and when a2 is 0, *—(L₂)ₐ₂—*′ is a single bond,
R_{ET} is a substituted or unsubstituted C₁-C₆₀ nitrogen-containing heterocyclic group,
R₁ to R₅ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), —B(Q₆)(Q₇), —P(═O)(Q₈)(Q₉), and —C(═O)(Q₁₀),
b1 and b2 are each independently an integer from 0 to 4, and b3 and b4 are each independently an integer from 0 to 3,
n1 is an integer from 1 to 8,
in Formula 1-1, a substituted or unsubstituted pyridinyl group, a substituted or unsubstituted pyrimidinyl group, a substituted or unsubstituted pyrazinyl group, a substituted or unsubstituted pyrazinyl group, and a substituted or unsubstituted triazinyl group are excluded from R_{ET} when A₁ and A₂ are each independently a substituted or unsubstituted C₁-C₆₀ alkyl group,
at least one substituent of the substituted C₁-C₆₀ nitrogen-containing heterocyclic group, the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group is selected from:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), —P(═O)(Q₁₈)(Q₁₉), and —C(═O)(Q₂₀);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), —P(═O)(Q₂₈)(Q₂₉), and —C(═O)(Q₃₀); and
—N(Q₃₁)(Q₃₂), —Si(Q₃₃)(Q₃₄)(Q₃₅), —B(Q₃₆)(Q₃₇), —P(═O)(Q₃₈)(Q₃₉), and —C(═O)(Q₄₀), and
Q₁ to Q₁₀, Q₁₁ to Q₂₀, Q₂₁ to Q₃₀, and Q₃₁ to Q₄₀ are each independently selected from hydrogen, deuterium, —F, —CI, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one selected from a C₁-C₆₀ alkyl group and a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

2. The heterocyclic compound of claim 1, wherein
R_{ET} is selected from:
an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a benzophenanthrolinyl group, a phenazinyl group, a benzimidazolyl group, a naphthoimidazolyl group, a benzothiazolyl group, an isobenzothiazolyl group, a benzoxazolyl group, an isobenzoxazolyl group, a triazolyl group, a tetrazolyl group, an oxadiazolyl group, an imidazopyridinyl group, an imidazopyrimidinyl group, an imidazoquinolinyl group, an imidazoisoquinolinyl group, an azadibenzofuranyl group, a diazadibenzofuranyl group, an azadibenzothiophenyl group, a diazadibenzothiophenyl group, an azafluorenyl group, a diazafluorenyl group, an azaspirobifluorenyl group, and a diazaspirobifluorenyl group;
an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a benzophenanthrolinyl group, a phenazinyl group, a benzimidazolyl group, a naphthoimidazolyl group, a benzothiazolyl group, an isobenzothiazolyl group, a benzoxazolyl group, an isobenzoxazolyl group, a triazolyl group, a tetrazolyl group, an oxadiazolyl group, an imidazopyridinyl group, an imidazopyrimidinyl group, an imidazoquinolinyl group, an imidazoisoquinolinyl group, an azadibenzofuranyl group, a diazadibenzofuranyl group, an azadibenzothiophenyl group, a diazadibenzothiophenyl group, an azafluorenyl group, a diazafluorenyl group, an azaspirobifluorenyl group, and a diazaspirobifluorenyl group, each substituted with at least one selected from deuterium, —F, —Cl, —Br, —I, a hydroxyl group, a cyano group, a nitro group, an amidino group, a hydrazine group, a hydrazone group, a C₁-C₁₀ alkyl group, a C₁-C₁₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a triphenylenyl group, a fluoranthenyl group, a pyrenyl group, a chrysenyl group, a picenyl group, a perylenyl group, a benzosilolyl group, a dibenzofuranyl group, a dibenzothiophenyl group, a pyridinyl group, a pyrazinyl group, a pyrimidinyl group, a pyridazinyl group, and a triazinyl group; and
an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, a triazinyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a benzophenanthrolinyl group, a phenazinyl group, a benzimidazolyl group, a naphthoimidazolyl group, a benzothiazolyl group, an isobenzothiazolyl group, a benzoxazolyl group, an isobenzoxazolyl group, a triazolyl group, a tetrazolyl group, an oxadiazolyl group, an imidazopyridinyl group, an imidazopyrimidinyl group, an imidazoquinolinyl group, an imidazoisoquinolinyl group, an azadibenzofuranyl group, a diazadibenzofuranyl group, an azadibenzothiophenyl group, a diazadibenzothiophenyl group, an azafluorenyl group, a diazafluorenyl group, an azaspirobifluorenyl group, and a diazaspirobifluorenyl group, each substituted with at least one selected from a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a triphenylenyl group, a fluoranthenyl group, a pyrenyl group, a chrysenyl group, a picenyl group, a perylenyl group, a benzosilolyl group, a dibenzofuranyl group, a dibenzothiophenyl group, a pyridinyl group, a pyrazinyl group, a pyrimidinyl group, a pyridazinyl group, and a triazinyl group, which are each substituted with at least one selected from a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a triphenylenyl group, a fluoranthenyl group, a pyrenyl group, a chrysenyl group, a picenyl group, a perylenyl group, a benzosilolyl group, a dibenzofuranyl group, a dibenzothiophenyl group, a pyridinyl group, a pyrazinyl group, a pyrimidinyl group, a pyridazinyl group, and a triazinyl group.

3. The heterocyclic compound of claim 1, wherein
R_{ET} is selected from groups represented by Formulae 2-1 to 2-3: 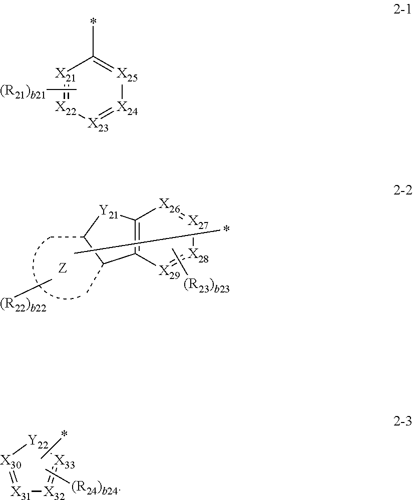
wherein, in Formulae 2-1 to 2-3,
X₂₁ to X₃₃ are each independently N or C,
at least one selected from X₂₁ to X₂₅ is N, at least one selected from X₂₆ to X₂₉ is N, and at least one selected from X₃₀ to X₃₃ is N,
Y₂₁ and Y₂₂ are each independently selected from O, S, N(R₂₅), and C(R₂₅)(R₂₆),
Z is selected from a substituted or unsubstituted C₅-C₃₀ carbocyclic group and a substituted or unsubstituted C₂-C₃₀ heterocyclic group,
R₂₁ to R₂₆ are each independently selected from hydrogen, deuterium, —F, —CI, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group,
two neighboring substituents of R₂₁ to R₂₆ are optionally linked to form a saturated ring or an unsaturated ring,
b21 to b23 are each independently an integer from 1 to 4, and b24 is an integer from 1 to 3, and
* indicates a binding site to a neighboring atom.

4. The heterocyclic compound of claim 3, wherein
R_{ET} is selected from groups represented by Formulae 3-1 to 3-18: 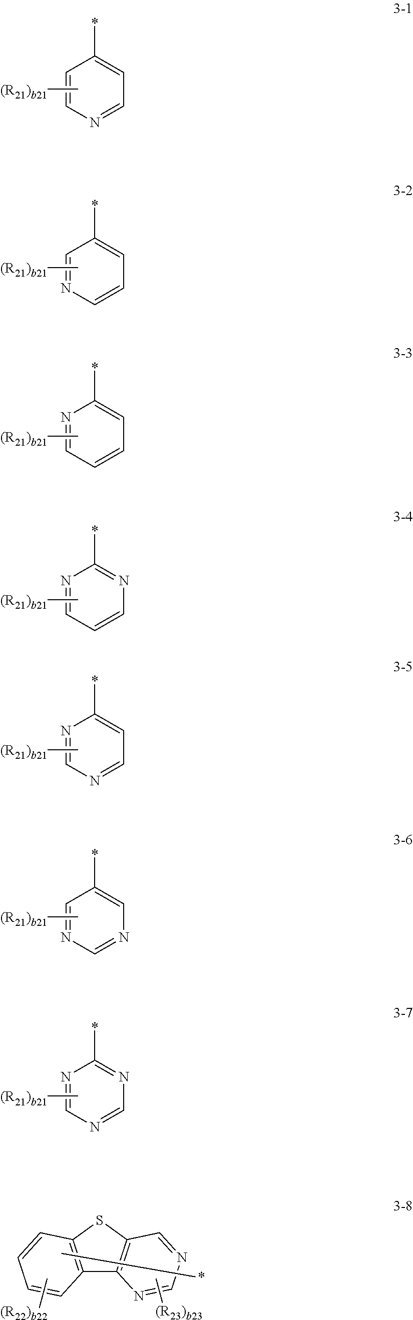 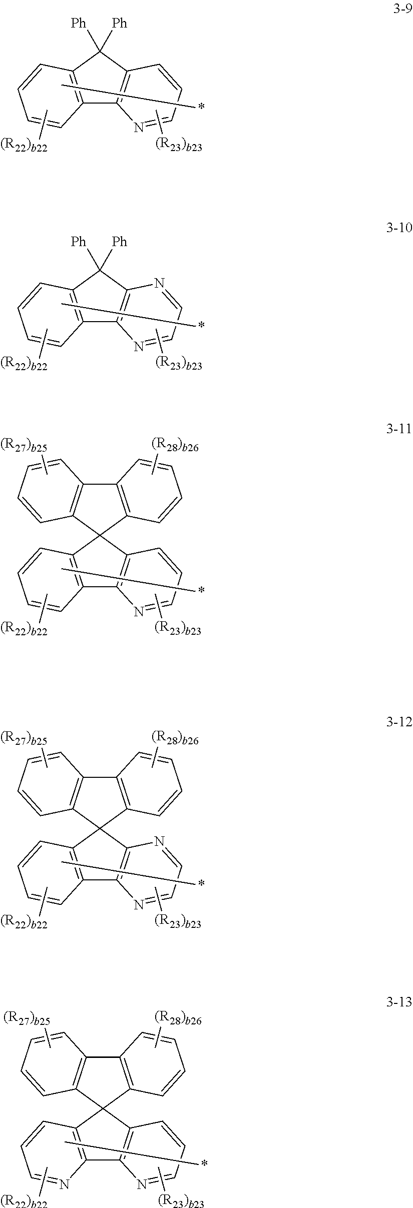 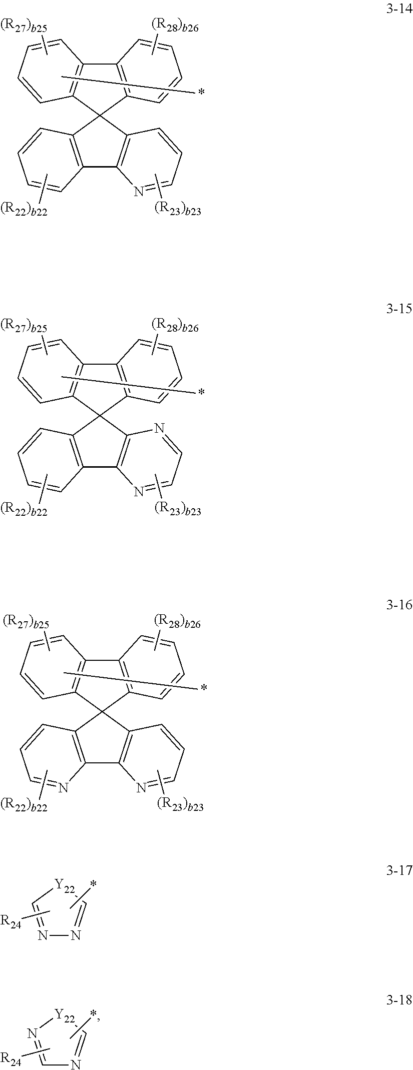
wherein, in Formulae 3-1 to 3-18,
R₂₁ to R_{24,} b21 to b23, and Y₂₂ are each independently the same as described in Formulae 2-1 to 2-3,
R₂₇ and R₂₈ are each independently the same as described in connection with R₂₁,
b25 and b26 are each independently an integer from 1 to 4, and
“Ph” indicates a phenyl group, and * indicates a binding site to a neighboring atom.

5. The heterocyclic compound of claim 3, wherein
R₂₁ to R₂₆ are each independently selected from hydrogen, deuterium, and Formulae R-1 to R-28: 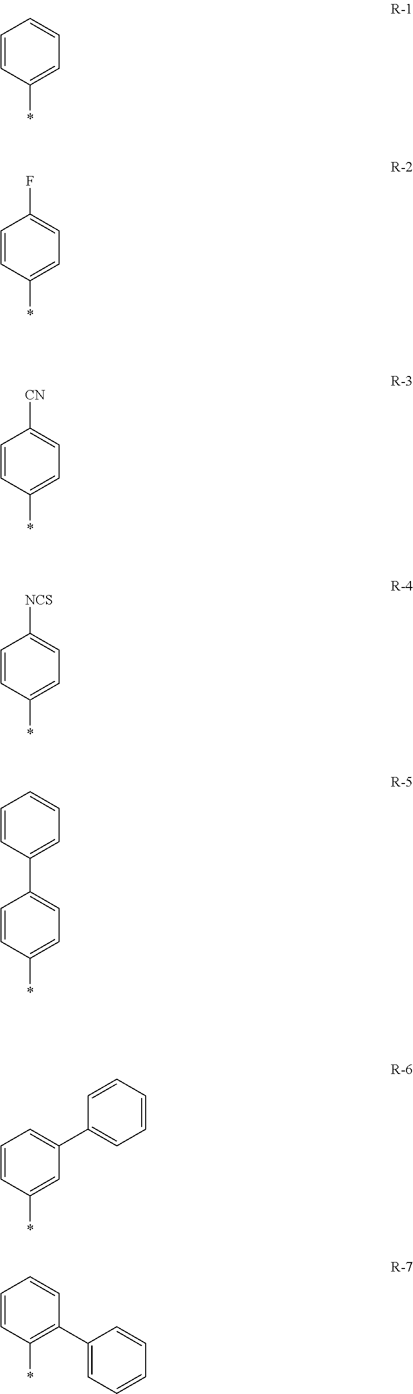 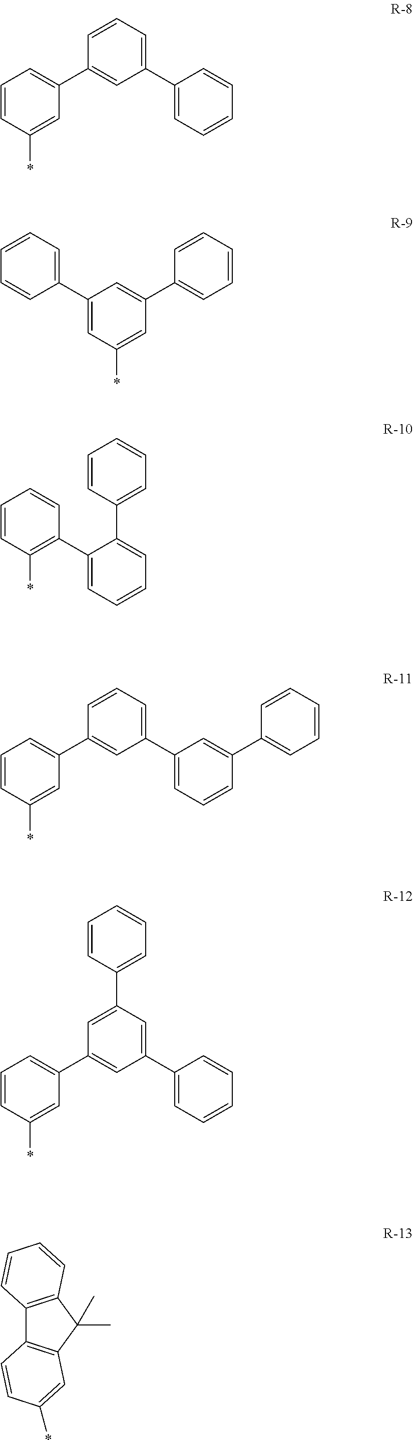 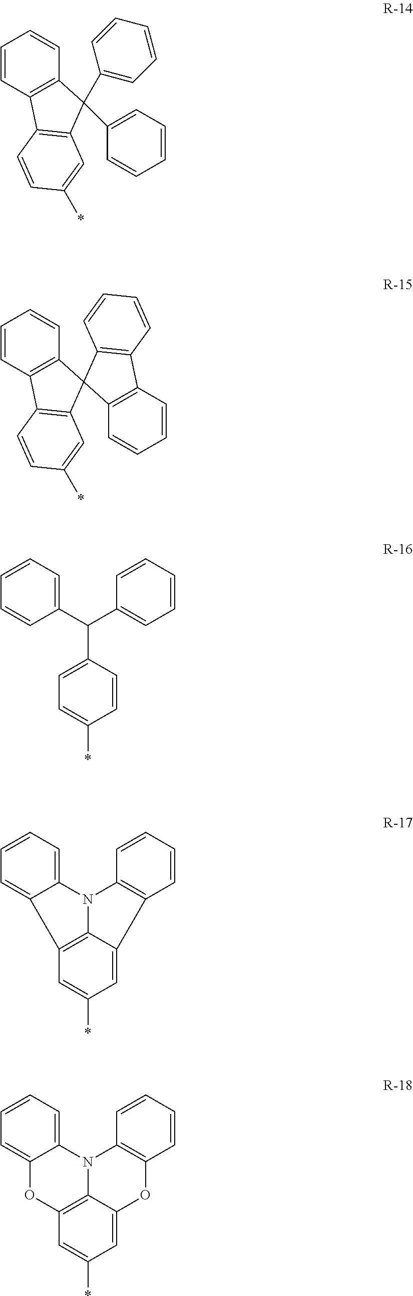 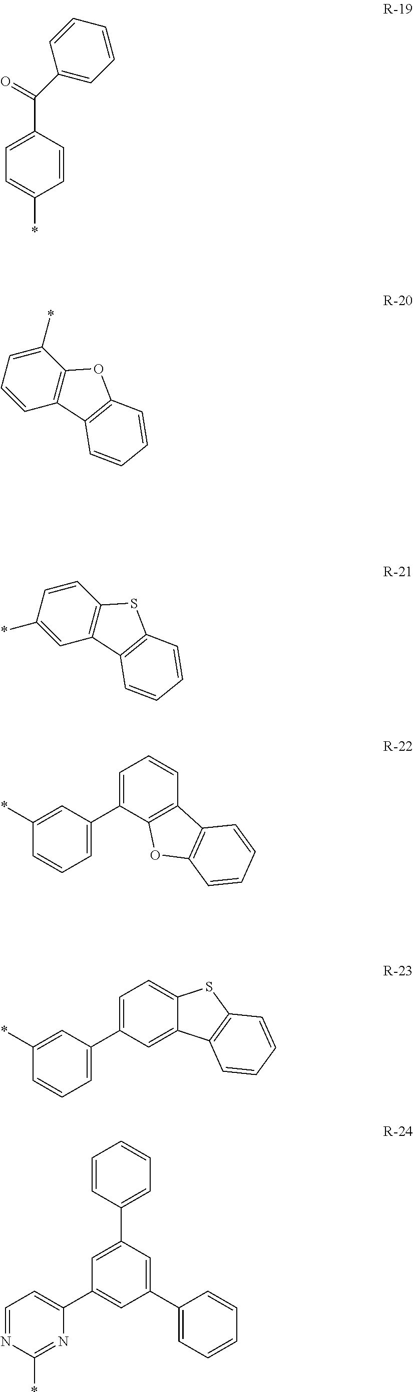 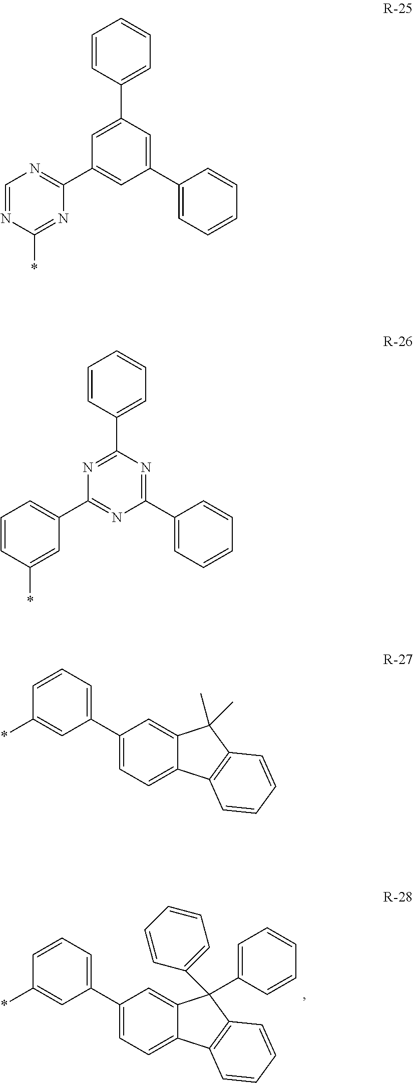
wherein, in Formulae R-1 to R-28,
* indicates a binding site to a neighboring atom.

6. The heterocyclic compound of claim 5, wherein
R₂₁ to R₂₆ are each independently selected from hydrogen, deuterium, and Formulae R-1, R-9, R-12 to R-15, and R-20 to R-28.

7. The heterocyclic compound of claim 1, wherein
A₁ to A₄ are each independently selected from:
hydrogen, a group represented by *—(L₂)ₐ₂—R_{ET}, a methyl group, an ethyl group, an n-propyl group, an iso-propyl group, an n-butyl group, an iso-butyl group, a sec-butyl group, a tert-butyl group, an n-pentyl group, an iso-pentyl group, a tert-pentyl group, a neo-pentyl group, a 1,2-dimethylpropyl group, an n-hexyl group, an iso-hexyl group, a 1,3-dimethylbutyl group, a 1-iso-propylpropyl group, a 1,2-dimethylbutyl group, an n-heptyl group, a 1,4-dimethylpentyl group, a 3-ethylpentyl group, a 2-methyl-1-iso-propylpropyl group, a 1-ethyl-3-methylbutyl group, an n-octyl group, a 2-ethylhexyl group, a 3-methyl-1-iso-propylbutyl group, a 2-methyl-1-iso-propyl group, a 1-tert-butyl-2-methylpropyl group, an n-nonyl group, a 3,5,5-trimethyldecyl group, an n-decyl group, an isodecyl group, an n-undecyl group, a 1-methyldecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, an n-eicosyl group, an n-heneicosyl group, an n-docosyl group, an n-tricosyl group, an n-tetracosyl group, a methoxy group, an ethoxy group, a propoxy group, an iso-propoxy group, an n-butoxy group, an iso-butoxy group, a sec-butoxy group, a tert-butoxy group, an n-pentoxy group, an iso-pentoxy group, a tert-pentoxy group, a neo-pentoxy group, an n-hexyloxy group, an iso-hexyloxy group, a heptyloxy group, an octyloxy group, a nonyloxy group, a decyloxy group, an undeoxy group, a dodecyloxy group, a tridecyloxy group, a tetradecyloxy group, a pentadecyloxy group, a hexadecyloxy group, a heptadecyloxy group, an octadecyloxy group, a 2-ethylhexyloxy group, a 3-ethylpentyloxy group, a phenoxy group, a 1-naphthyloxy group, a 2-naphthyloxy group, a 2-azulenyloxy group, a 2-furanyloxy group, a 2-thienyloxy group, a 2-indoleyloxy group, a 3-indoleyloxy group, a 2-benzofuriloxy group, a 2-benzothienyloxy group, an N-methylamino group, an N-ethylamino group, an N-propylamino group, an N-iso-propylamino group, an N-butylamino group, an N-iso-butylamino group, an N-sec-butylamino group, an N-tert-butylamino group, an N-pentylamino group, an N-hexylamino group, an N,N-dimethylamino group, an N,N-diethylamino group, an N,N-dipropylamino group, an N,N-di-iso-propylamino group, an N,N-dibutylamino group, an N,N-di-iso-butylamino group, an N,N-dipentylamino group, and an N,N-dihexylamino group;
a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group; and
a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

8. The heterocyclic compound of claim 1, wherein
A₁ and A₂ are linked to form a group represented by Formula 9: 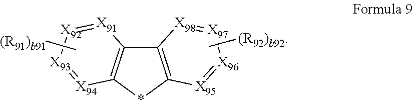
wherein, in Formula 9,
X₉₁ to X₉₈ are each independently N or C,
R₉₁ to R₉₂ are each independently selected from hydrogen, deuterium, —F, —CI, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), and —B(Q₆)(Q₇),
b91 to b92 are each independently an integer from 1 to 4, and
* indicates a binding site to a neighboring atom.

9. The heterocyclic compound of claim 1, wherein
L₁ and L₂ are each independently selected from:
a benzene group, a pentalene group, an indene group, a naphthalene group, an anthracene group, an azulene group, a heptalene group, an acenaphthylene group, a phenalene group, a fluorene group, a spiro-bifluorene group, a benzofluorene group, a dibenzofluorene group, a phenanthrene group, biphenyl group, terphenyl group, a triphenylene group, a fluoranthene group, a pyrene group, a chrysene group, a picene group, a perylene group, a pentaphene group, a pentacene group, a tetraphene group, a hexaphene group, a hexacene group, a rubicene group, a trinaphthalene group, a heptaphene group, a pyranthrene group, a pyridine group, a pyrazine group, a pyridazine group, a pyrimidine group, a triazine group, a quinoline group, an isoquinoline group, a quinoxaline group, a quinazoline group, a naphthyridine group, an acridine group, a phenazine group, a benzoquinoline group, a benzoisoquinoline group, a phenanthridine group, a phenanthroline group, a benzoquinone group, a coumarin group, an anthraquinone group, a fluorenone group, a furan group, a thiophene group, a silole group, a benzofuran group, a benzothiophene group, a benzosilole group, a dibenzofuran group, a dibenzothiophene group, a dibenzosilole group, a pyrrole group, an indole group, an isoindole group, a carbazole group, a benzocarbazole group, a dibenzocarbazole group, an imidazole group, a benzimidazole group, a pyrazole group, a triazole group, a tetrazole group, an indazole group, an oxazole group, an isoxazole group, a benzoxazole group, a benzoisoxazole group, a thiazole group, an isothiazole group, a benzothiazole group, a benzoisothiazole group, an imidazolinone group, a benzoimidazolinone group, an imidazopyridine group, an imidazopyrimidine group, an imidazophenanthridine group, a benzoimidazophenanthridine group, an azadibenzofuran group, an azacarbazole group, an azadibenzothiophene group, a diazadibenzofuran group, a diazacarbazole group, a diazadibenzothiophene group, a xanthone group, and a thioxanthone group; and
a benzene group, a pentalene group, an indene group, a naphthalene group, an anthracene group, an azulene group, a heptalene group, an acenaphthylene group, a phenalene group, a fluorene group, a spiro-bifluorene group, a benzofluorene group, a dibenzofluorene group, a phenanthrene group, biphenyl group, terphenyl group, a triphenylene group, a fluoranthene group, a pyrene group, a chrysene group, a picene group, a perylene group, a pentaphene group, a pentacene group, a tetraphene group, a hexaphene group, a hexacene group, a rubicene group, a trinaphthalene group, a heptaphene group, a pyranthrene group, a pyridine group, a pyrazine group, a pyridazine group, a pyrimidine group, a triazine group, a quinoline group, an isoquinoline group, a quinoxaline group, a quinazoline group, a naphthyridine group, an acridine group, a phenazine group, a benzoquinoline group, a benzoisoquinoline group, a phenanthridine group, a phenanthroline group, a benzoquinone group, a coumarin group, an anthraquinone group, a fluorenone group, a furan group, a thiophene group, a silole group, a benzofuran group, a benzothiophene group, a benzosilole group, a dibenzofuran group, a dibenzothiophene group, a dibenzosilole group, a pyrrole group, an indole group, an isoindole group, a carbazole group, a benzocarbazole group, a dibenzocarbazole group, an imidazole group, a benzimidazole group, a pyrazole group, a triazole group, a tetrazole group, an indazole group, an oxazole group, an isoxazole group, a benzoxazole group, a benzoisoxazole group, a thiazole group, an isothiazole group, a benzothiazole group, a benzoisothiazole group, an imidazolinone group, a benzoimidazolinone group, an imidazopyridine group, an imidazopyrimidine group, an imidazophenanthridine group, a benzoimidazophenanthridine group, an azadibenzofuran group, an azacarbazole group, an azadibenzothiophene group, a diazadibenzofuran group, a diazacarbazole group, a diazadibenzothiophene group, a xanthone group, and a thioxanthone group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

10. The heterocyclic compound of claim 1, wherein
R₁ to R₅ are each independently selected from:
hydrogen, deuterium, a methyl group, an ethyl group, an n-propyl group, an iso-propyl group, an n-butyl group, an iso-butyl group, a sec-butyl group, a tert-butyl group, an n-pentyl group, an iso-pentyl group, a tert-pentyl group, a neo-pentyl group, a 1,2-dimethylpropyl group, an n-hexyl group, an iso-hexyl group, a 1,3-dimethylbutyl group, a 1-iso-propylpropyl group, a 1,2-dimethylbutyl group, an n-heptyl group, a 1,4-dimethylpentyl group, a 3-ethylpentyl group, a 2-methyl-1-iso-propylpropyl group, a 1-ethyl-3-methylbutyl group, an n-octyl group, a 2-ethylhexyl group, a 3-methyl-1-iso-propylbutyl group, a 2-methyl-1-iso-propyl group, a 1-tert-butyl-2-methylpropyl group, an n-nonyl group, a 3,5,5-trimethyldecyl group, an n-decyl group, an isodecyl group, an n-undecyl group, a 1-methyldecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, an n-eicosyl group, an n-heneicosyl group, an n-docosyl group, an n-tricosyl group, an n-tetracosyl group, a methoxy group, an ethoxy group, a propoxy group, an iso-propoxy group, an n-butoxy group, an iso-butoxy group, a sec-butoxy group, a tert-butoxy group, an n-pentoxy group, an iso-pentoxy group, a tert-pentoxy group, a neo-pentoxy group, an n-hexyloxy group, an iso-hexyloxy group, a heptyloxy group, an octyloxy group, a nonyloxy group, a decyloxy group, an undeoxy group, a dodecyloxy group, a tridecyloxy group, a tetradecyloxy group, a pentadecyloxy group, a hexadecyloxy group, a heptadecyloxy group, an octadecyloxy group, a 2-ethylhexyloxy group, a 3-ethylpentyloxy group, a phenoxy group, a 1-naphthyloxy group, a 2-naphthyloxy group, a 2-azulenyloxy group, a 2-furanyloxy group, a 2-thienyloxy group, a 2-indoleyloxy group, a 3-indoleyloxy group, a 2-benzofuriloxy group, a 2-benzothienyloxy group, an N-methylamino group, an N-ethylamino group, an N-propylamino group, an N-iso-propylamino group, an N-butylamino group, an N-iso-butylamino group, an N-sec-butylamino group, an N-tert-butylamino group, an N-pentylamino group, an N-hexylamino group, an N,N-dimethylamino group, an N,N-diethylamino group, an N,N-dipropylamino group, an N,N-di-iso-propylamino group, an N,N-dibutylamino group, an N,N-di-iso-butylamino group, an N,N-dipentylamino group, and an N,N-dihexylamino group;
a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group; and
a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group, each substituted with at least one selected from deuterium, —F, —Cl, —Br, —I, a hydroxyl group, a cyano group, a nitro group, an amidino group, a hydrazine group, a hydrazone group, a C₁-C₃₀ alkyl group, a C₁-C₃₀ alkoxy group, a phenyl group, a pentalenyl group, an indenyl group, a naphthyl group, an anthracenyl group, an azulenyl group, a heptalenyl group, an acenaphthyl group, a phenalenyl group, a fluorenyl group, a spiro-bifluorenyl group, a benzofluorenyl group, a dibenzofluorenyl group, a phenanthrenyl group, a biphenyl group, a terphenyl group, a triphenylenyl group, a fluoranthenyl group, a pyrenyl group, a chrysenyl group, a picenyl group, a perylenyl group, a pentaphenyl group, a pentacenyl group, a tetraphenyl group, a hexaphenyl group, a hexacenyl group, a rubicenyl group, a trinaphthyl group, a heptaphenyl group, a pyranthrenyl group, a pyridinyl group, a pyrazinyl group, a pyridazinyl group, a pyrimidinyl group, a triazinyl group, a quinolinyl group, an isoquinolinyl group, a quinoxalinyl group, a quinazolinyl group, a naphthyridinyl group, an acridinyl group, a phenazinyl group, a benzoquinolinyl group, a benzoisoquinolinyl group, a phenanthridinyl group, a phenanthrolinyl group, a benzoquinonyl group, a cumarinyl group, an anthraquinonyl group, a fluorenonyl group, a furanyl group, a thienyl group, a silolyl group, a benzofuranyl group, a benzothienyl group, a benzosilolyl group, a dibenzofuranyl group, a dibenzothiophenyl group, a dibenzosilolyl group, a pyrrolyl group, an indolyl group, an isoindolyl group, a carbazolyl group, a benzocarbazolyl group, a dibenzocarbazolyl group, an imidazolyl group, a benzimidazolyl group, a pyrazolyl group, a triazolyl group, a tetrazolyl group, an indazolyl group, an oxazolyl group, an isoxazolyl group, a benzoxazolyl group, a benzisoxazolyl group, a thiazolyl group, an isothiazolyl group, a benzothiazolyl group, a benzisothiazolyl group, an imidazopyridinyl group, an imidazopyrimidinyl group, an imidazophenanthridinyl group, a benzimidazophenanthridinyl group, an azadibenzofuranyl group, an azacarbazolyl group, an azadibenzothienyl group, a diazadibenzofuranyl group, a diazacarbazolyl group, a diazadibenzothienyl group, a xanthonyl group, and a thioxanthonyl group.

11. The heterocyclic compound of claim 1, wherein,
in Formula 1-1, a position of carbons to which A₃ is linked is asymmetric to a position of carbons to which A₄ is linked, and
in Formula 1-2, a position of carbons to which —(L₁)ₐ₁— is linked is asymmetric to a position of carbons to which A₄ is linked.

12. The heterocyclic compound of claim 1, wherein,
in Formula 1-1, X₁₂ is carbon linked to A₃, X₁₄ is carbon linked to A₄, A₃ is a group represented by *—(L₂)ₐ₂—R_{ET}, and A₄ is a substituted or unsubstituted C₆-C₆₀ aryl group, and
in Formula 1-2, X₁₁ is carbon linked to —(L₁)ₐ₁—, X₁₃ is carbon linked to A₄, and A₄ is a group represented by *—(L₂)ₐ₂—R_{ET}.

13. The heterocyclic compound of claim 1, wherein
the heterocyclic compound has a molecular weight of about 850 grams per mole to about 3,000 grams per mole.

14. A composition comprising the heterocyclic compound of claim 1.

15. The composition of claim 14, further comprising at least one selected from a compound represented by Formula 4-1, a compound represented by Formula 4-2, a compound represented by Formula 5, a compound represented by Formula 6, and a compound represented by Formula 7: 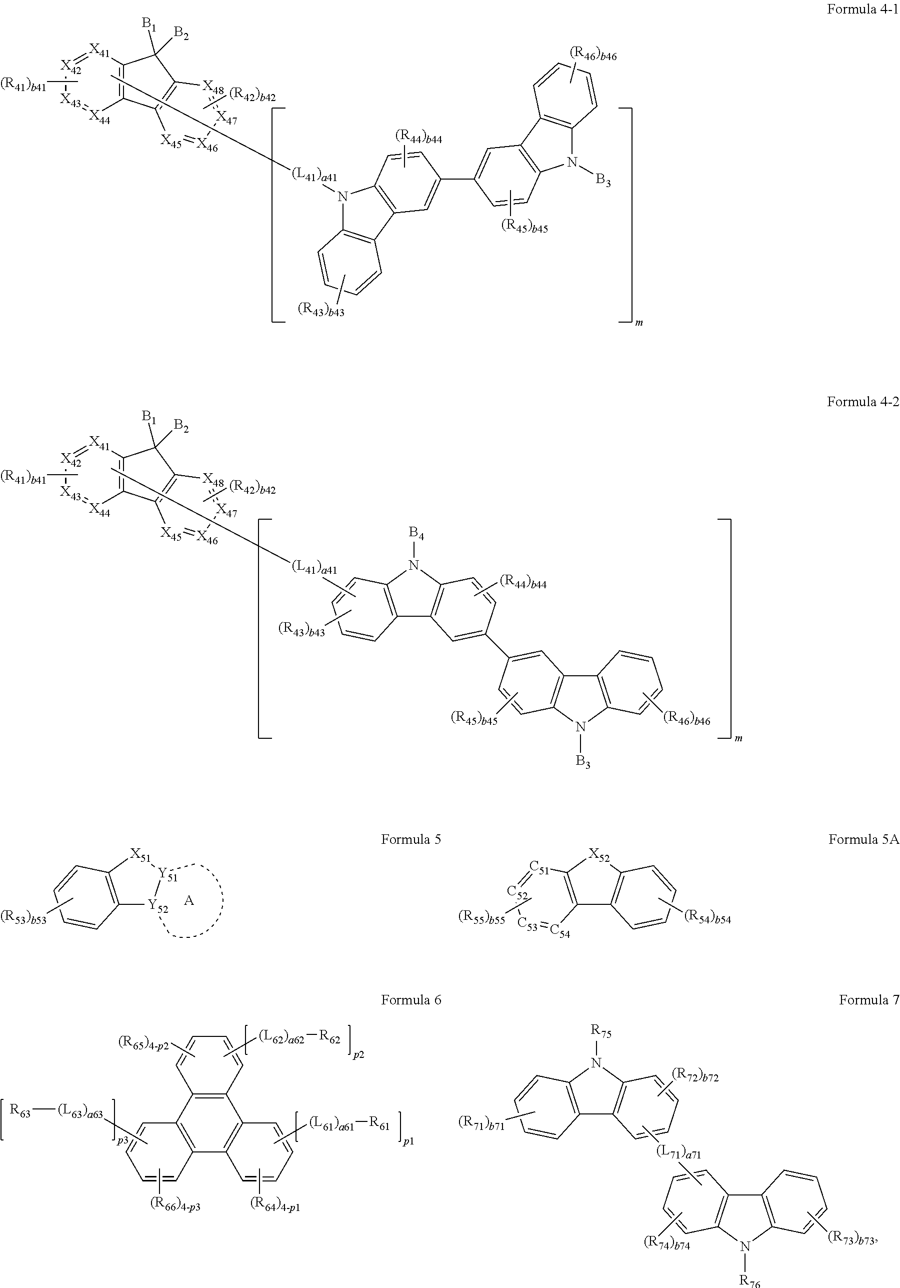
wherein, in Formulae 4-1, 4-2, 5, 5A, 6, and 7,
X₄₁ to X₄₈ are each independently N or C,
at least one selected from X₄₁ to X₄₈ is carbon linked to —(L₄₁)ₐ₄₁—,
X₅₁ is N(R₅₁), O, or S, and X₅₂ is N(R₅₂), O, or S,
C₅₁ to C₅₄ are each a carbon atom (C), and two neighboring groups selected from C₅₁ to C₅₄ are condensed with a group represented by Formula 5 via Y₅₁ and Y₅₂,
L₄₁, L₆₁ to L₆₃ and L₇₁ are each independently selected from a substituted or unsubstituted C₅-C₃₀ carbocyclic group and a substituted or unsubstituted C₁-C₃₀ heterocyclic group,
a41, a61 to a63, and a71 are each independently an integer from 0 to 3,
A is a group represented by Formula 5A,
R₄₁ to R₄₆, R₅₁ to R₅₅, R₆₁ to R₆₆, and R₇₁ to R₇₆ are each independently selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —Si(Q₁)(Q₂)(Q₃), —N(Q₄)(Q₅), and —B(Q₆)(Q₇),
b41 to b43, b46, b53 to b54, and b71 to b73 are each independently an integer from 0 to 4,
b44, b45, b72, and b74 are each independently an integer from 0 to 3,
b55 is an integer from 0 to 2,
B₁ and B₄ are each independently selected from a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group,
m is an integer from 1 to 8, and
p1 to p3 are each independently an integer from 0 to 4.

16. An organic light-emitting device comprising:
a first electrode;
a second electrode; and
an organic layer disposed between the first electrode and the second electrode, wherein the organic layer comprises an emission layer, and
wherein the organic layer comprises at least one heterocyclic compound of claim 1.

17. The organic light-emitting device of claim 16, wherein
the first electrode is an anode,
the second electrode is a cathode, and
the organic layer further comprises a hole transport region disposed between the first electrode and the emission layer and an electron transport region disposed between the emission layer and the second electrode,
wherein the hole transport region comprises at least one selected from a hole injection layer, a hole transport layer, a buffer layer, and an electron blocking layer, and
wherein the electron transport region comprises at least one selected from a hole blocking layer, an electron transport layer, and an electron injection layer.

18. The organic light-emitting device of claim 16, wherein
the emission layer comprises the heterocyclic compound.

19. The organic light-emitting device of claim 18, wherein
the emission layer further comprises a material capable of emitting light from triplet excitons.

20. The organic light-emitting device of claim 17, wherein
the hole transport region comprises an amine-based polymer.
